(19) United States
(12) Patent Application Publication
(10) Pub. No.: US 2022/0339416 A1
(43) Pub. Date: Oct. 27, 2022

(54) SILK FIBROIN-BASED MICRONEEDLES AND USES THEREOF

(71) Applicant: VAXESS TECHNOLOGIES, INC., Cambridge, MA (US)

(72) Inventors: Kathryn M. Kosuda, Cambridge, MA (US); Kimberly M. Cirelli, Brookline, MA (US); Alexander Beliveau, Cambridge, MA (US); Archana V. Boopathy, San Mateo, CA (US); Matthew Dirckx, Somerville, MA (US); Jonathan A. Kluge, Cambridge, MA (US); Michael A. Schrader, Watertown, MA (US); Megan Eisele, New York, NY (US)

(73) Assignee: VAXESS TECHNOLOGIES, INC., Cambridge, MA (US)

(21) Appl. No.: 17/714,025

(22) Filed: Apr. 5, 2022

Related U.S. Application Data
(63) Continuation of application No. PCT/US2020/055139, filed on Oct. 9, 2020.
(60) Provisional application No. 62/912,832, filed on Oct. 9, 2019.

(51) Int. Cl. A61M 37/00; A61K 9/00; A61K 47/42; A61P 35/00; A61K 45/06

(52) U.S. Cl. CPC A61M 37/0015; A61K 9/0021; A61K 47/42; A61P 35/00; A61K 45/06

(57) ABSTRACT

Microneedle and microneedle devices including, e.g., silk fibroin-based microneedles tips for sustained dermal delivery of an anti-cancer agent and/or an immunomodulatory agent, as well as methods of manufacturing and using the same are described herein. In other embodiments, compositions and methods for burst-release or sustained-release administration of an anti-cancer agent and/or an immunomodulatory agent to provide an improved immune response to a cancer in a subject are described.

### RELATED APPLICATIONS

This application is a continuation of International Patent Application No.: PCT/US2020/055139, filed Oct. 9, 2020; which claims the benefit of U.S. Provisional Application No. 62/912,832, filed Oct. 9, 2019. The entire contents of each of the foregoing applications are hereby incorporated herein by reference.

### FIELD OF THE INVENTION

The present disclosure generally relates to silk fibroin-based microneedles configured to release a therapeutic agent, such as an anti-cancer agent, an immunomodulatory agent, or a combination thereof, to treat a subject having a disease or disorder, e.g., a cancer.

### BACKGROUND

Drug delivery into the skin for local or systemic effects is extremely difficult due to the highly effective barrier properties of the skin's outermost layer, the stratum corneum. Microneedle devices comprise sub-millimeter needles designed to be minimally invasive and to by-pass the stratum corneum to access the skin's microcirculation to achieve local and/or systemic delivery of a therapeutic agent by the transdermal route. However, traditional microneedle designs and materials are associated with various limitations that compromise their production and limit their performance (see, e.g., Donnelly et al. Drug Deliv. 17(4): 187-207, 2010). Specifically, it is difficult to design a microneedle that has both the sufficient mechanical strength to pierce the stratum corneum and the capacity to incorporate, and subsequently release, an effective amount of a therapeutic agent. There exists a need for improved microneedle designs and fabrication processes.

### SUMMARY

The present disclosure is based, at least in part, on the realization that silk fibroin has suitable properties for use in microneedle fabrication, including all-aqueous processing, mechanical strength, biocompatibility, and the ability to stabilize and control the release of various therapeutic agents from a silk-based matrix. Additionally, the present disclosure is based, at least in part, on the realization that local administration of a therapeutic agent, such as an anti-cancer agent and/or an immunomodulatory agent, to the site of a tumor can result in an inhibition of tumor growth at or near the site of administration and also can result in a systemic immune response to ablate tumors at distant sites. Further, that certain diseases, such as cancer, are associated with tumor-specific antigens (e.g., neoantigens), which are poorly and/or ineffectively presented to a subject's immune system by antigen presenting cells (APCs), e.g., due to the immunosuppressive tumor microenvironment (TME). Without wishing to be bound by theory, the present disclosure provides silk fibroin-based microneedles, and silk fibroin-based microneedle devices, which can be used, for example, to alter the tumor microenvironment by administering an effective amount of an anti-cancer agent, an immunomodulatory agent, or a combination thereof, thereby facilitating the presentation of tumor-specific antigens (e.g., neoantigens) to APCs in a subject's body and the development of a robust and long-lasting immunity to the tumor-specific antigens (e.g., cancer immunity). In some embodiments, the silk fibroin-based microneedles, and silk fibroin-based microneedle devices, can be used to deliver a patient-specific neoantigen (e.g., a cancer vaccine) to achieve enhanced immunity via sustained release of a therapeutic agent, such as an anti-cancer agent and/or an immunomodulatory agent, in a subject.

The present disclosure provides silk fibroin-based microneedles, and silk fibroin-based microneedle devices, configured to incorporate, and subsequently release (e.g., administer), an effective amount of a therapeutic agent or a combination of therapeutic agents to a subject (e.g., a human subject). In some embodiments, the silk fibroin-based microneedles, and silk fibroin-based microneedle devices comprise an anti-cancer agent, an immunomodulatory agent, or a combination thereof. In some embodiments, the microneedles disclosed herein can be used in combination with a second therapeutic agent or procedure, such as a cancer therapy (e.g., one or more of anti-cancer agents, immunotherapy, photodynamic therapy (PDT), surgery and/or radiation).

The disclosed silk fibroin-based microneedles can be configured to release a therapeutic agent or a combination of therapeutic agents (e.g., an anti-cancer agent, an immunomodulatory agent, or a combination thereof) according to various release kinetics, such as burst release (e.g., immediate or quick dissolution of the microneedle upon application to a biological barrier, such as skin, mucous surface, tumor, oral cavity, or buccal cavity, usually occurring within minutes), and/or sustained release. Examples of sustained release include, but are not limited to, zero order release (e.g., the rate of release is independent of the therapeutic agent concentration in the dosage form, e.g., the release rate is approximately constant over a period of time, e.g., a constant amount of therapeutic agent is eliminated per unit time), first order release (e.g., the rate of release is a function of the amount of the therapeutic agent remaining in the dosage form, e.g., a constant proportion, such as a percentage, of drug is eliminated per unit time), and second order release (e.g., where doubling the concentration of therapeutic agent in the dosage for quadruples the release rate). In some embodiments, a portion of a microneedle is configured for a first type of release, e.g., burst release, and another portion of the microneedle is configured for a second type of release, e.g., sustained release. Further, the release (e.g., administration) of a therapeutic agent (e.g., an anti-cancer agent, an immunomodulatory agent, or a combination thereof) from a silk fibroin-based microneedle described herein can be facilitated by the diffusion of the therapeutic agent from the microneedle or a portion thereof; the degradation (e.g., protease mediated degradation) of the microneedle or a portion thereof; and/or the dissolution of the microneedle or a portion thereof.

The disclosed silk fibroin-based microneedles can be configured to have sufficient mechanical properties (e.g., strength) and suitable geometry (e.g., tip sharpness, tip included angle, length, inter-needle spacing) to pierce a biological barrier (e.g., skin, tumor, tissue, a cell membrane, a mucous surface, an oral cavity, or a buccal cavity) to achieve a local and/or a systemic delivery of a therapeutic agent or a combination of therapeutic agents (e.g., an anti-cancer agent, an immunomodulatory agent, or a combination thereof) to a subject. In some embodiments, a microneedle or device described herein is configured to administer a therapeutic agent (e.g., an anti-cancer agent, an immunomodulatory agent, or a combination thereof) to a site of a tumor (e.g., intratumorally and/or peritumorally). In some embodiments, a microneedle or device described herein is configured to administer a therapeutic agent (e.g., an anti-cancer agent, an immunomodulatory agent, or a combination thereof) to the site of a skin lesion.

In some embodiments, local administration of a therapeutic agent (e.g., an anti-cancer agent, an immunomodulatory agent, or a combination thereof) to the site of a tumor and/or a skin lesion by a silk fibroin-based microneedle, or silk fibroin-based microneedle device, described herein can result in an anti-cancer effect (e.g., inhibition of tumor growth) at or near the site of administration, and optionally can result in a systemic anti-cancer effect (e.g., an immune response) to ablate tumors at distant sites.

In some embodiments, the silk fibroin-based microneedles, and silk fibroin-based microneedle devices are administered in combination with a standard of care treatment (e.g., for a cancer or skin condition), optionally chosen from a surgery, a chemotherapy, an immunotherapy, a targeted therapy, a hormone therapy, and/or a radiation therapy.

Also disclosed are methods of manufacturing and using such microneedles for treating a disease, for example, a cancer, in a subject.

Those skilled in the art will recognize, or be able to ascertain using no more than routine experimentation, many equivalents to the specific embodiments of the present disclosure described herein. Such equivalents are intended to be encompassed by the following embodiments (E).

E1. A microneedle device (e.g., a microneedle patch) comprising a plurality of silk fibroin-based microneedles, wherein said plurality of microneedles comprises:

a first microneedle comprising an anti-cancer agent; and

a second microneedle comprising an immunomodulatory agent,

optionally, a third microneedle comprising an anti-cancer agent and/or an immunomodulatory agent,

wherein the first and/or second microneedles comprises a silk fibroin (e.g., regenerated silk fibroin and/or recombinant silk fibroin), and the microneedle device is configured to deliver to a subject the anti-cancer agent and the immunomodulatory agent.

E2. A microneedle device (e.g., a microneedle patch) comprising a plurality of silk fibroin-based microneedles, wherein said plurality of microneedles comprises:

two or more microneedles comprising an anti-cancer agent,

wherein the two or more microneedles comprises a silk fibroin (e.g., regenerated silk fibroin and/or recombinant silk fibroin), and the microneedle device is configured to deliver to a subject the anti-cancer agent.

E3. A microneedle device (e.g., a microneedle patch) comprising a plurality of silk fibroin-based microneedles, wherein said plurality of microneedles comprises:

two or more microneedles comprising an immunomodulatory agent,

wherein the two or more microneedles comprises a silk fibroin (e.g., regenerated silk fibroin and/or recombinant silk fibroin), and the microneedle device is configured to deliver to a subject the immunomodulatory agent, optionally, wherein the immunomodulatory agent enhances an immune response against a cancer.

E4. The microneedle device of any one of the preceding embodiments, wherein the first and/or second microneedle in the plurality of microneedles comprises:

(i) a base, (e.g., a dissolvable base),

(ii) a silk fibroin tip (e.g., an implantable silk fibroin tip) comprising a silk fibroin applied to the base, and

(iii) (optional) a backing applied to the base.

E5. A plurality of microneedles, wherein said plurality of microneedles comprises:

a first microneedle comprising an anti-cancer agent; and

a second microneedle comprising an immunomodulatory agent,

optionally, a third microneedle comprising an anti-cancer agent and/or an immunomodulatory agent,

wherein the first and/or second microneedle comprises a silk fibroin, e.g., a regenerated silk fibroin and/or a recombinant silk fibroin.

E6. A plurality of microneedles, wherein said plurality of microneedles comprises:

a first microneedle comprising an anti-cancer agent; and

a second microneedle comprising an anti-cancer agent,

wherein the first and/or second microneedle comprises a silk fibroin, e.g., a regenerated silk fibroin and/or a recombinant silk fibroin.

E7. A plurality of microneedles, wherein said plurality of microneedles comprises:

a first microneedle comprising an immunomodulatory agent; and

a second microneedle comprising an immunomodulatory agent,

wherein the first and/or second microneedle comprises a silk fibroin, e.g., a regenerated silk fibroin and/or a recombinant silk fibroin.

E8. The microneedle device of embodiment E4, wherein the silk fibroin tip comprises the anti-cancer agent and/or the immunomodulatory agent.
E9. The microneedle device of embodiment E4, wherein the base comprises the anti-cancer agent and/or the immunomodulatory agent.
E10. The microneedle device, or the plurality of microneedles, of any one of the preceding embodiments, wherein the microneedle is configured to pierce a biological barrier (e.g., skin).
E11. The microneedle device, of embodiment E4 or E8, wherein microneedle is configured to implant the silk fibroin tip into a biological barrier (e.g., skin) of a subject.
E12. The microneedle device of any one of embodiments E1-E4 or E8-E11, wherein the microneedle device is configured to achieve a local and/or a systemic delivery (e.g., release) of the anti-cancer agent and/or the immunomodulatory agent to the subject.
E13. The microneedle device of any one of embodiments E1-E4 or E8-E12, wherein the microneedle device is configured to deliver an effective amount of the anti-cancer agent and/or the immunomodulatory agent to the subject.
E14. The microneedle device of embodiment E4 or E8, wherein the silk fibroin tip comprises a regenerated silk fibroin and/or a recombinant silk fibroin.
E15. The microneedle device, or the plurality of microneedles, of any one of the preceding embodiments, which is configured to deliver (e.g., release) two or more anti-cancer agents (e.g., three or more, four or more, or five or more anti-cancer agents).
E16. The microneedle device, or the plurality of microneedles, of any one of the preceding embodiments, which is configured to deliver (e.g., release) two or more immunomodulatory agents (e.g., three or more, four or more, or five or more immunomodulatory agents).
E17. The microneedle device, or the plurality of microneedles, of any one of the preceding embodiments, wherein said plurality of microneedles comprises at least one additional microneedle, wherein the additional microneedle comprises the same first anti-cancer agent.
E18. The microneedle device, or the plurality of microneedles, of any one of the preceding embodiments, wherein said plurality of microneedles comprises at least one additional microneedle, wherein the additional microneedle comprises an anti-cancer agent different from the first anti-cancer agent (“a second anti-cancer agent”).
E19. The microneedle device, or the plurality of microneedles, of any one of the preceding embodiments, wherein said plurality of microneedles comprises at least one additional microneedle, wherein the additional microneedle comprises the same first immunomodulatory agent.
E20. The microneedle device, or the plurality of microneedles, of any one of the preceding embodiments, wherein said plurality of microneedles comprises at least one additional microneedle, wherein the additional microneedle comprises an immunomodulatory agent that is different from the first immunomodulatory agent (“a second immunomodulatory agent”).
E21. The microneedle device, or the plurality of microneedles, of any one of the preceding embodiments, wherein said plurality of microneedles comprises an additional silk fibroin-based microneedle comprising a second anti-cancer agent.
E22. The microneedle device, or the plurality of microneedles, of any one of the preceding embodiments, wherein said plurality of microneedles comprises an additional silk fibroin-based microneedle comprising a second immunomodulatory agent.
E23. The microneedle device, or the plurality of microneedles, of any one of the preceding embodiments, comprising a plurality of said first, second, and/or additional microneedles.
E24. The microneedle device, or the plurality of microneedles, of any one of embodiments E1, E2, E4-E6, or E8-E23, wherein the anti-cancer agent is chosen from one or more of a small molecule (e.g., a chemotherapy drug), a biologic (e.g., an antibody), a viral cancer therapeutic agent, a nanopharmaceutical, and a nucleic acid molecule (e.g., DNA and/or RNA).
E25. The microneedle device, or the plurality of microneedles, of any one of embodiments E1, E2, E4-E6, or E8-E24, wherein the anti-cancer agent is not a chemotherapeutic nucleotide.
E26. The microneedle device, or the plurality of microneedles, of any one of embodiments E1, E2, E4-E6, or E8-E25, wherein the anti-cancer agent is an mRNA, optionally wherein the mRNA encodes an anti-cancer agent and/or an immunomodulatory agent, optionally wherein the mRNA encodes a checkpoint inhibitor, a TLR agonist, a STING agonist, a RIG agonist, a cancer vaccine, a targeted therapy, and/or a cytokine.
E27. The microneedle device, or the plurality of microneedles, of any one of embodiments E1, E2, E4-E6, or E8-E26, wherein the anti-cancer agent is chosen from one or more of Gemcitabine (GEMZAR®), Vemurafenib (ZELBORAF®), Dabrafenib (TAFINLAR®), Trametinib (MEKINIST®), Doxorubicin (ADRIAMYCIN®), Encorafenib (BRAFTOVI®), Cobimetinib (COTELLIC®), Binimetinib (MEKTOVI®), Dacarbazine (DTIC), Temozolomide (TEMODAR®), Ipilimumab (YERVOY®), Pembrolizumab (KEYTRUDA®), Nivolumab (OPDIVO®), Aldesleukin (Proleukin®), Recombinant Interferon Alfa-2b (Intron A), Peginterferon Alfa-2b (PEG-Intron/Sylatron), oxaliplatin, and Talimogene Laherparepvec (IMLYGIC®).
E28. The microneedle device, or the plurality of microneedles, of any one of embodiments E1, E3-E5, or E7-E27, wherein the immunomodulatory agent is chosen from a checkpoint inhibitor, a Toll-like receptor (TLR) agonist, a STING agonist, a RIG agonist, a cancer vaccine, and a cytokine.
E29. The microneedle device, or the plurality of microneedles, of embodiment E28, wherein the checkpoint inhibitor inhibits a checkpoint molecule chosen from CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR.
E30. The microneedle device, or the plurality of microneedles, of embodiment E28 or E29, wherein the checkpoint inhibitor is a PD-1 inhibitor.
E31. The microneedle device, or the plurality of microneedles, of embodiment E28 or E29, wherein the checkpoint inhibitor is a CTLA4 inhibitor.
E32. The microneedle device, or the plurality of microneedles, of embodiment E28, wherein the TLR agonist is chosen from a TLR-1 agonist, a TLR-2 agonist, a TLR-3 agonist, a TLR-4 agonist, a TLR-5 agonist, a TLR-6 agonist, a TLR-7 agonist, a TLR-8 agonist, a TLR-9 agonist, a TLR-10 agonist, a TLR-1/2 agonist, a TLR-2/6 agonist, or a TLR-7/8 agonist.
E33. The microneedle device, or the plurality of microneedles, of embodiment E28 or E32, wherein the TLR agonist is a TLR-7 agonist.
E34. The microneedle device, or the plurality of microneedles, of embodiment E28 or E32, wherein the TLR agonist is a TLR-9 agonist (e.g., an unmethylated CG dinucleotides (CpG ODN).
E35. The microneedle device, or the plurality of microneedles, of embodiment E28, wherein the STING agonist is cyclic dinucleotide, e.g., a cyclic dinucleotide comprising purine or pyrimidine nucleobases (e.g., adenosine, guanine, uracil, thymine, or cytosine nucleobases), optionally bis-(3′-5′)-cyclic dimeric guanosine monophosphate (c-di-GMP).
E36. The microneedle device, or the plurality of microneedles, of embodiment E28, wherein the cytokine is GM-CSF, IL-1α, IL-10, IL-2, IL-3, IL-4, IL-5, IL-6, IL-7, IL-8, IL-10, IL-12, IL-15, IL-21, IFN-α, IFN-β, IFN-γ, MIP-1α, MIP-1β, TGF-β, TNF-α, and TNFβ.
E37. The microneedle device, or the plurality of microneedles, of embodiment E28, wherein the cytokine is GM-CSF, IL-1α, IL-10, IL-2, IL-3, IL-4, IL-5, IL-6, IL-7, IL-8, IL-10, IL-12, IL-15, IL-18, IL-21, IFN-α, IFN-β, IFN-γ, MIP-1α, MIP-1β, TGF-β, TNF-α, or TNFβ.
E38. The microneedle device, or the plurality of microneedles, of embodiment E28 or E36, wherein the cytokine is IL-2.
E39. The microneedle device, or the plurality of microneedles, of embodiment E28 or E36, wherein the cytokine is IL-12.
E40. The microneedle device, or the plurality of microneedles, of embodiment E28 or E36, wherein the cytokine is IL-15.
E41. The microneedle device, or the plurality of microneedles, of embodiment E28 or E37, wherein the cytokine is IL-18.
E42. The microneedle device, or the plurality of microneedles, of any one of embodiments E28, E36, or E37, wherein the cytokine is an engineered cytokine.
E43. The microneedle device, or the plurality of microneedles, of any one of embodiments E28, E36, or E37, wherein the cytokine is an engineered interleukin (e.g., engineered IL-2 or IL-18).
E44. The microneedle device, or the plurality of microneedles, of any one of embodiments E28, E36, or E37, wherein the cytokine is an engineered interleukin 2.
E45. The microneedle device, or the plurality of microneedles, of embodiment E28 or E37, wherein the cytokine is an engineered interleukin 18.
E46. The microneedle device, or the plurality of microneedles, of any one of embodiments E28, E36, or E37, wherein the cytokine is a decoy-resistant interleukin.
E47. The microneedle device, or the plurality of microneedles, of embodiment E28 or E37, wherein the cytokine is a decoy-resistant interleukin 18.
E48. The microneedle device, or the plurality of microneedles, of embodiment E28 or E36, wherein the cytokine is GM-CSF.
E49. The microneedle device, or the plurality of microneedles, of any one of the preceding embodiments, which is configured to administer an anti-PD1 antibody and/or an anti-CTLA4 antibody in combination with one or more of:

(i) IL-2;

(ii) IL-12;

(iii) IL-15;

(iv) Gemcitabine (GEMZAR®);

(v) Vemurafenib (ZELBORAF®);

(vi) Dabrafenib (TAFINLAR®);

(vii) Trametinib (MEKINIST®);

(viii) Doxorubicin (ADRIAMYCIN®);

(ix) c-di-GMP;

(x) mRNA;

(xi) a TLR-9 agonist (e.g., an unmethylated CG dinucleotides (CpG ODN));

(xii) oxaliplatin; and

(xiii) GM-CSF.

E50. The microneedle device, or the plurality of microneedles, of any one of the preceding embodiments, which is configured to administer an anti-PD1 antibody and/or an anti-CTLA4 antibody in combination with one or more of:

(i) IL-2;

(ii) IL-12;

(iii) IL-15;

(iv) IL-18

(v) Gemcitabine (GEMZAR®);

(vi) Vemurafenib (ZELBORAF®);

(vii) Dabrafenib (TAFINLAR®);

(viii) Trametinib (MEKINIST®);

(ix) Doxorubicin (ADRIAMYCIN®);

(x) c-di-GMP;

(xi) mRNA;

(xii) a TLR-9 agonist (e.g., an unmethylated CG dinucleotides (CpG ODN));

(xiii) oxaliplatin; and

(xiv) GM-CSF.

E51. The microneedle device, or the plurality of microneedles, of embodiments E28 or E37, which is configured to administer an anti-PD1 antibody and/or an anti-CTLA4 antibody in combination with one or more of IL-2, IL-12, or IL-18.
E52. The microneedle device, or the plurality of microneedles, of any one of the preceding embodiments, which is configured to administer an anti-PD1 antibody and/or an anti-CTLA4 antibody in combination with IL-2 and IL-12.
E53. The microneedle device, or the plurality of microneedles, of any one of the preceding embodiments, which is configured to administer an anti-PD1 antibody and/or an anti-CTLA4 antibody in combination with IL-2 and a TLR-9 agonist (e.g., an unmethylated CG dinucleotides (CpG ODN)).
E54. The microneedle device, or the plurality of microneedles, of any one of the preceding embodiments, which is configured to administer IL-2 and c-di-GMP, optionally IL-12.
E55. The microneedle device, or the plurality of microneedles, of any one of the preceding embodiments, which is configured to administer IL-12 and c-di-GMP, optionally IL-2.
E56. The microneedle device, or the plurality of microneedles, of any one of the preceding embodiments, which is configured to administer a cancer vaccine, optionally wherein the cancer vaccine comprises a tumor antigen, e.g., a neoantigen.
E57. A microneedle comprising:

(i) a base, (e.g., a dissolvable base),

(ii) an implantable silk fibroin tip comprising a silk fibroin applied or attached to the base, and

(iii) (optional) a backing applied to the base,

wherein the microneedle is configured to implant the silk fibroin tip into a biological barrier (e.g., skin) of a subject, e.g., a human subject,

wherein the silk fibroin tip comprises a silk fibroin, e.g., a regenerated silk fibroin and/or a recombinant silk fibroin,

wherein the silk fibroin tip further comprises an anti-cancer agent in an amount sufficient to induce an anti-cancer response.

E58. A microneedle device, or a plurality of microneedles, comprising the microneedle of embodiment E57, optionally, wherein at least two microneedles of the plurality comprise the same anti-cancer agent or a different anti-cancer.
E59. A microneedle comprising:

(i) a base, (e.g., a dissolvable base),

(ii) an implantable silk fibroin tip comprising a silk fibroin applied to the base, and

(iii) (optional) a backing applied to the base,

wherein the microneedle is configured to implant the silk fibroin tip into a biological barrier (e.g., skin) of a subject, e.g., a human subject,

wherein the silk fibroin tip comprises a silk fibroin, e.g., a regenerated silk fibroin and/or a recombinant silk fibroin,

wherein the silk fibroin tip further comprises an immunomodulatory agent in an amount sufficient to stimulate and/or suppress the immune system.

E60. A microneedle device, or a plurality of microneedles, of any one of embodiments E1, E3-E5, E7-E56, or E59, optionally, wherein at least two microneedles of the plurality comprise the same immunomodulatory agent or a different immunomodulatory agent.
E61. A microneedle device comprising a plurality of silk fibroin-based microneedles, wherein said plurality of microneedles comprises one or more microneedles of any one of the preceding embodiments.
E62. The microneedle device, the plurality of microneedles, or the microneedle of any one of the preceding embodiments, wherein the device, plurality, or microneedle is configured for sustained release of the anti-cancer agent and/or the immunomodulatory agent.
E63. The microneedle device, the plurality of microneedles, or the microneedle of embodiment E62, wherein the sustained release comprises a substantially continuous low dose administration of the anti-cancer agent and/or the immunomodulatory agent.
E64. The microneedle device, the plurality of microneedles, or the of embodiment E62 or E63, wherein the sustained release comprises a continuous administration of about a greater than 0% portion to about a 100% portion of a total amount of anti-cancer agent and/or a total amount of immunomodulatory agent present in the silk fibroin tip.
E65. The microneedle device, the plurality of microneedles, or the microneedle of any one embodiments E62-E64, wherein the sustained release is over a period of time comprising at least about 3 days (e.g., about 3, 4, 5, 6, 7, or more days, e.g., between about 5 days and about 10 days, e.g., between about 7 days and about 15 days, e.g., between about 1 to about 2 weeks, between about 1 to about 3 weeks, or between about 2 to about 4 weeks, e.g., between about 1 to about 3 months, e.g., between about 2 to about 4 months, e.g., between about 3 to about 6 months).
E66. The microneedle device, the plurality of microneedles, or the microneedle of any one of embodiments E62-E65, wherein the sustained release is over a period of time between about 2 days and about 28 days.
E67. The microneedle device, the plurality of microneedles, or the microneedle of any one of embodiments E62-E66, wherein the sustained release is over a period of time between about 5 days and about 21 days.
E68. The microneedle device, the plurality of microneedles, or the microneedle of any one of the preceding embodiments, which is configured to release an effective amount of the anti-cancer agent and/or the immunomodulatory agent to enhance exposure of the subject's immune system to a neoantigen associated with a cancer (e.g., a neoantigen released after tumor cell lysis), thereby inducing and/or expanding immune effector cells, e.g., T cells, that are specific for the neoantigen.
E69. The microneedle device, the plurality of microneedles, or the microneedle of any one of the preceding embodiments, which is configured to release an effective amount of the anti-cancer agent and/or the immunomodulatory agent to induce T cell activation and/or to overcome immunosuppression in a tumor microenvironment.
E70. The microneedle device, the plurality of microneedles, or the microneedle of any one of the preceding embodiments, wherein the device, plurality, or microneedle is configured for burst release of the anti-cancer agent and/or the immunomodulatory agent.
E71. The microneedle device, the plurality of microneedles, or the microneedle of embodiment E70, wherein the burst release comprises a rapid administration of the anti-cancer agent and/or the immunomodulatory agent.
E72. The microneedle device, the plurality of microneedles, or the microneedle of embodiment E70 or E71, wherein the burst release comprises a rapid administration of a greater than 0% portion to about a 100% portion of a total amount of anti-cancer agent and/or a total amount of immunomodulatory agent present in the silk fibroin tip.
E73. The microneedle device, the plurality of microneedles, or the microneedle of any one of embodiments E70-E72, wherein the burst release is over a period of time comprising at least about 1 hour (e.g., about 1 to about 30 minutes, e.g., about 1, 2, 3, 4, 5, 6, 7, 8, 9, 10, 15, 20, or 24 hours).
E74. The microneedle device, the plurality of microneedles, or the microneedle of any one of embodiments E12, E15, E16, or E62-E73, wherein the release of the anti-cancer agent occurs at substantially the same rate (e.g., concurrently) with the release of the immunomodulatory agent.
E75. The microneedle device, the plurality of microneedles, or the microneedle of any one of embodiments E12, E15, E16, or E62-E74, wherein the release of the anti-cancer agent occurs at a different rate than the release of the immunomodulatory agent, such that anti-cancer agent is released substantially before or substantially after the release of the immunomodulatory agent.
E76. The microneedle device, the plurality of microneedles, or the microneedle of any one of the preceding embodiments, configured to be applied (e.g., administered) to a biological barrier selected from a layer of skin, a cell membrane, a mucous surface, an oral cavity, or a buccal cavity.
E77. The microneedle device, the plurality of microneedles, or the microneedle of any one of the preceding embodiments, configured to be applied (e.g., administered) to a tumor (e.g., a metastatic tumor).
E78. The microneedle device, the plurality of microneedles, or the microneedle of any one of the preceding embodiments, configured to be applied (e.g., administered) to be applied to the site of a tumor after tumor resection, e.g., to induce and immune response to the tumor and/or to ablate any cancer cells left behind after resection.
E79. The microneedle device, the plurality of microneedles, or the microneedle of any one of the preceding embodiments, configured to be applied (e.g., administered) to be applied to the site of a tumor before tumor resection, e.g., to induce an immune response to the tumor and/or to ablate any cancer cells left behind after resection.
E80. The microneedle device, the plurality of microneedles, or the microneedle of any one of the preceding embodiments, configured to be applied (e.g., administered) to the skin.
E81. The microneedle device, the plurality of microneedles, or the microneedle of any one of the preceding embodiments, configured to be applied (e.g., administered) to the eye.
E82. The microneedle device, the plurality of microneedles, or the microneedle of any one of the preceding embodiments, configured to be applied (e.g., administered) to the oral cavity.
E83. The microneedle device, the plurality of microneedles, or the microneedle of any one of the preceding embodiments, configured to be applied (e.g., administered) intratumorally.
E84. The microneedle device, the plurality of microneedles, or the microneedle of any one of the preceding embodiments, configured to be applied (e.g., administered) peritumorally.
E85. The microneedle device, the plurality of microneedles, or the microneedle of any one of the preceding embodiments, configured to be applied (e.g., administered) to, or proximal to, a skin lesion (e.g., a skin lesion associated with a cancer or a precancerous condition).
E86. The microneedle device of embodiment E12, wherein the local and/or systemic delivery (e.g., release) results in:

(i) inhibition of tumor growth at or near the site of administration;

(ii) an induction of a local immune response to ablate tumors at or near the site of administration;

(iii) an increase in activated immune effector cells (e.g., T cells) in the tumor microenvironment;

(iv) a reduction in local immunosuppressive cells (e.g., regulatory T cells (Tregs));

(v) an induction of a systemic immune response to ablate tumors at distant sites;

(vi) an immunological memory to the cancer or precancerous condition; and/or

(vii) an immune response to a tumor antigen, such as a neoantigen; and/or

(viii) prevention and/or inhibition of cancer recurrence (e.g., cancer relapse).

E87. The microneedle device, the plurality of microneedles, or the microneedle of any one of embodiments E4 or E57-E59, wherein the backing is chosen from a solid support, e.g., a paper-based material, a plastic material, a polymeric material, or a polyester-based material (e.g., a Whatman 903 paper, a polymeric tape, a plastic tape, an adhesive-backed polyester tape, or other medical tape).
E88. The microneedle device, the plurality of microneedles, or the microneedle of any one of embodiments E4, E9, E57-E59, or E87, wherein the base (e.g., dissolvable base) comprises two or more of:

(i) a polysaccharide (e.g., dextran);

(ii) a disaccharide (e.g., sucrose, maltose, and trehalose);

(iii) a polymer (e.g., methyl cellulose, polyethylene glycol (PEG), carboxymethylcellulose (CMC), polyvinylpyrrolidone (PVP), polyvinyl alcohol (PVA), and hyaluronate);

(iv) a protein (e.g., gelatin);

(v) a plasticizer (e.g., glycerol, propanediol); and

(vi) a surfactant (e.g., an octyl phenol ethoxylate (e.g., Triton-X), a polysorbate, a poloxamer, and/or a polyethoxylated alcohol).

E89. The microneedle device, the plurality of microneedles, or the microneedle of any one of embodiments E4, E9, E57-E59, E87, or E88, wherein the base comprises one or more of gelatin, dextran, glycerol, polyethylene glycol (PEG) (e.g., including low molecular weight PEG), sucrose, trehalose, maltose, carboxymethylcellulose (CMC), polyvinylpyrrolidone (PVP), polyvinyl alcohol (PVA), hyaluronate, methyl cellulose, and/or a surfactant (e.g., an octyl phenol ethoxylate (e.g., Triton-X), a polysorbate, a poloxamer, such as P188, and/or a polyethoxylated alcohol), optionally wherein the microneedle is configured for sustained release and/or burst release.
E90. The microneedle device, the plurality of microneedles, or the microneedle of any one of embodiments E4, E9, E57-E59, or E87-E89, wherein the base comprises dextran, sucrose, glycerol, and a surfactant, optionally configured for sustained release.
E91. The microneedle device, the plurality of microneedles, or the microneedle of any one of embodiments E62-E67, E89, or E90, which is configured for sustained release comprises (optionally wherein this is the solution used for casting and/or the composition of the dried, solidified base):

(i) between about 20% to about 40%, e.g., 30%, 70 kDa Dextran;

(ii) between about 5% to about 15%, e.g., about 10%, Sucrose;

(iii) between about 0.5% to about 2.5%, e.g., about 1%, Glycerol; and

(iv) between about 0.001% to about 1%, e.g., about 0.01%, Triton-X.

E92. The microneedle device, the plurality of microneedles, or the microneedle of any one embodiments E88-E91, wherein the dextran has a molecular weight of between about 30 kD and about 600 kDa.
E93. The microneedle device, the plurality of microneedles, or the microneedle of any one of embodiments E88-E92, wherein the dextran is derived from Leuconostoc mesenteroides.
E94. The microneedle device, the plurality of microneedles, or the microneedle of any one of embodiments E4, E8, E9, E57-E59, E87, or E88-E93, wherein the base comprises polyvinyl alcohol (PVA) and sucrose, optionally configured for burst release.
E95. The microneedle device, the plurality of microneedles, or the microneedle of any one of embodiments E70-E72, E89, or E94, which is configured for burst release comprises (optionally wherein this is the solution used for casting and/or the composition of the dried, solidified base):

(i) about 15% to about 20%, e.g., about 18%, PVA;

(ii) about 25% to about 75% sucrose, e.g., about 50% sucrose;

(iii) about 25% to about 75% PVA, e.g., about 50% PVA; and

(iv) about 15% to about 20%, e.g., about 18%, sucrose.

E96. The microneedle device, the plurality of microneedles, or the microneedle of any one of embodiments E4, E8, E9, E57-E59, E87, or E88-E95, wherein the base does not comprise poly(acrylic acid) (PAA).
E97. The microneedle device, the plurality of microneedles, or the microneedle of any one of embodiments E4, E8, E9-E96, wherein the silk fibroin tip comprises two or more of:

(i) a disaccharide (e.g., sucrose, maltose, and trehalose);

(ii) a polymer (e.g., methyl cellulose, polyethylene glycol (PEG), carboxymethylcellulose (CMC), polyvinylpyrrolidone (PVP), polyvinyl alcohol (PVA), hyaluronate);

(iii) an amino acid (e.g., threonine);

(iv) a plasticizer (e.g., glycerol, propanediol); and

(v) a buffer (e.g., PBS).

E98. The microneedle device, the plurality of microneedles, or the microneedle of any one of embodiments E4, E8, E9-E97, wherein the silk fibroin tip comprises an excipient.
E99. The microneedle device, the plurality of microneedles, or the microneedle of any one of embodiments E4, E8, E9-E98, wherein the silk fibroin tip comprises one or more of carboxymethylcellulose (CMC), sucrose, and threonine.
E100. The microneedle device, the plurality of microneedles, or the microneedle of any one of embodiments E4, E8, E9-E99, wherein the silk fibroin tip comprises a buffer, optionally phosphate buffered saline (PBS).
E101. The microneedle device, the plurality of microneedles, or the microneedle of any one of embodiments E70-E72, E89, E94, or E95, wherein the silk fibroin tip configured for burst-release comprises between about 2% to about 8% sucrose (e.g., about 5% sucrose).
E102. The microneedle device, the plurality of microneedles, or the microneedle of any one of embodiments E70-E72, E89, E94, E95, or E101, wherein the silk fibroin tip configured for burst-release comprises about 0.5% to about 3% w/v CMC (e.g., about 1% CMC).
E103. The microneedle device, the plurality of microneedles, or the microneedle of any one of embodiments E70-E72, E89, E94, E95, E101, or E102, wherein the silk fibroin tip configured for burst-release comprises about 50 mM to about 100 mM of an amino acid, such as threonine (e.g., about 75 mM threonine).
E104. The microneedle device, the plurality of microneedles, or the microneedle of any one of embodiments E4, E8, or E9-E103, wherein the silk fibroin tip comprises silk fibroin at about 1% w/v to about 10% w/v (e.g., about 1, 2, 3, 4, 5, 6, 7, 8, 9, or 10% w/v, or a silk fibroin having a molecular weight distribution according to FIG. 5, or, comprises silk fibroin in an amount between about 20 μg to about 245 μg, e.g., per 121 microneedle array).
E105. The microneedle device, the plurality of microneedles, or the microneedle of any one of embodiments E4, E8, or E9-E103, wherein the silk fibroin tip comprises about 1% w/v to about 10% w/v (e.g., about 1, 2, 3, 4, 5, 6, 7, 8, 9, or 10% w/v) of 10 MB silk fibroin solution, or a silk fibroin solution according to FIG. 5.
E106. The microneedle device, the plurality of microneedles, or the microneedle of any one of embodiments E4, E8, or E9-E103, wherein the silk fibroin tip comprises about 1% w/v to about 10% w/v (e.g., about 1, 2, 3, 4, 5, 6, 7, 8, 9, or 10% w/v) of 60 MB silk fibroin solution, or a silk fibroin solution according to FIG. 5, e.g., a 100 kDa to 200 kDa (e.g., about 153 kDa) silk fibroin solution.
E107. The microneedle device, the plurality of microneedles, or the microneedle of any one of embodiments E4, E8, or E9-E103, wherein the silk fibroin tip comprises about 1% w/v to about 10% w/v (e.g., about 1, 2, 3, 4, 5, 6, 7, 8, 9, or 10% w/v) of 120 MB silk fibroin solution, or a silk fibroin solution according to FIG. 5, e.g., a 70 kDa to 150 kDa (e.g., about 100 kDa) silk fibroin solution.
E108. The microneedle device, the plurality of microneedles, or the microneedle of any one of embodiments E4, E8, or E9-E103, wherein the silk fibroin tip comprises about 1% w/v to about 10% w/v (e.g., about 1, 2, 3, 4, 5, 6, 7, 8, 9, or 10% w/v) of 180 MB silk fibroin solution, or a silk fibroin solution according to FIG. 5, e.g., a 36 kDa to 100 kDa (e.g., about 71 kDa) silk fibroin solution.
E109. The microneedle device, the plurality of microneedles, or the microneedle of any one of embodiments E4, E8, or E9-E103, wherein the silk fibroin tip comprises about 1% w/v to about 10% w/v (e.g., about 1, 2, 3, 4, 5, 6, 7, 8, 9, or 10% w/v) of 480 MB silk fibroin solution, or a silk fibroin solution according to FIG. 5, e.g., a 1 kDa to 60 kDa (e.g., about 16 kDa) silk fibroin solution.
E110. The microneedle device, the plurality of microneedles, or the microneedle of any one of embodiments E62-E67, E89, E90, or E91, wherein the silk fibroin tip configured for sustained-release comprises between about 1% to about 10% w/v of 60 MB silk fibroin solution.
E111. The microneedle device, the plurality of microneedles, or the microneedle of any one of embodiments E62-E67, E89, E90, or E91, wherein the silk fibroin tip configured for sustained-release comprises between about 1% to about 10% w/v of 60 MB silk fibroin solution.
E112. The microneedle device, the plurality of microneedles, or the microneedle of any one of embodiments E62-E67, E89, E90, or E91, wherein the silk fibroin tip configured for sustained-release comprises between about 1% to about 10% w/v of 120 MB silk fibroin solution.
E113. The microneedle device, the plurality of microneedles, or the microneedle of any one of embodiments E62-E67, E89, E90, or E91, wherein the silk fibroin tip configured for sustained-release comprises between about 1% to about 10% w/v of 180 MB silk fibroin solution.
E114. The microneedle device, the plurality of microneedles, or the microneedle of any one of embodiments E62-E67, E89, E90, or E91, wherein the silk fibroin tip configured for sustained-release comprises between about 1% to about 10% w/v of 480 MB silk fibroin solution.
E115. The microneedle device, the plurality of microneedles, or the microneedle of any one of the preceding embodiments, which comprises substantially only dissolvable materials, e.g., substantially only polymeric-based and/or or sugar-based materials.
E116. The microneedle device, the plurality of microneedles, or the microneedle of any one of the preceding embodiments, which is configured to be applied to the site of a tumor, e.g., post-resection, and left in place to fully dissolve.
E117. The microneedle device, the plurality of microneedles, or the microneedle of any one of embodiments E4, E8, or E9-E114, wherein the microneedle is configured to implant the silk fibroin tip into a biological barrier of a subject at a depth (e.g., a max penetration depth of the distal part of tip) of between about 100 μm and about 1 mm.
E118. The microneedle device, the plurality of microneedles, or the microneedle of any one of the preceding embodiments, wherein the length of the microneedle is between about 350 μm to about 1500 μm.
E119. The microneedle device, the plurality of microneedles, or the microneedle of any one of embodiments E4, E8, E9-E114, or E117, wherein the height of the silk fibroin tip may extend to approximately half of the full height of the microneedle.
E120. The microneedle device, the plurality of microneedles, or the microneedle of any one of embodiments E4, E8, E9-E114, or E117, wherein the height of the silk fibroin tip is between about 75 μm to about 475 μm.
E121. The microneedle device, the plurality of microneedles, or the microneedle of any one of embodiments E4, E8, E9-E114, or E117, wherein the silk fibroin tip comprises a tip radius between about 0.5 μm to about 100 μm.
E122. The microneedle device, the plurality of microneedles, or the microneedle of any one of embodiments E4, E8, E9-E114, or E117, wherein the silk fibroin tip comprises a tip radius between about 5 μm to about 10 μm.
E123. The microneedle device, the plurality of microneedles, of the microneedle of any one of embodiments E4, E8, E9-E114, or E117, wherein the silk fibroin tip comprises an angle between about 5 degrees and about 45 degrees.
E124. A method for treating and/or inducing an immune response to a cancer (e.g., a metastatic cancer), comprising contacting (e.g., administering) a microneedle device, or a plurality of microneedles, of any one of embodiments E1-E56, E58, or E60-E123, to the site of a cancer (e.g., a metastatic tumor) of a subject, thereby resulting in one or more of the following:

(i) lysis of a cancer cell, e.g., tumor cells, to release and/or to expose a cancer-associated antigen (e.g., a neoantigen) to the subject's immune system;

(ii) display by an antigen presenting cell (APC) to an immune system cell (e.g., an accessory cell, such as a B-cell, a dendritic cell, and the like) of a cancer-associated antigen (e.g., the neoantigen) complexed with a major histocompatibility complex (MHC) on its surface;

(iii) recognition of the displayed cancer-associated antigen (e.g., the neoantigen) by an immune effector cell, e.g., a T cell and/or an NK cell;

(iv) activation and/or expansion of an immune effector cell, e.g., a T cell and/or an NK cell, specific for the displayed cancer-associated antigen (e.g., a neoantigen) in the subject; and

(v) an enhanced, e.g., stimulated or up-regulated, immune response of an immune effector cells, e.g., a T cell and/or an NK cell, that promotes the killing of and/or the inhibition of the growth or the proliferation of a target cell expressing the cancer-associated antigen (e.g., a neoantigen) in the subject.

E125. The method of embodiment E124, wherein the cancer expresses a plurality of antigens (e.g., neoantigens) but in which the tumor microenvironment hinders the activation of immune effector cells, e.g., T cells, that recognize them.
E126. The method of embodiment E124 or E125, wherein the enhanced, e.g., stimulated or up-regulated, immune response to the target cell expressing the cancer-associated antigen (e.g., a neoantigen) occurs at or near the site of administration.
E127. The method of any one of embodiments E124-E126, wherein the enhanced, e.g., stimulated or up-regulated, immune response to the target cell expressing the cancer-associated antigen (e.g., a neoantigen) is a systemic immune response (e.g., a broad spectrum response) to ablate tumors at distant sites.
E128. A method for treating and/or inducing an immune response to a cancer (e.g., a metastatic cancer), comprising contacting (e.g., administering) a microneedle device, or a plurality of microneedles, of any one of embodiments E1-E56, E58, or E60-E123, to the site of a cancer (e.g., a metastatic tumor) of a subject
E129. A method for treating a cancer (e.g., a metastatic cancer), or a precancerous condition (e.g., a precancerous skin condition), comprising contacting (e.g., administering) a microneedle device, or a plurality of microneedles, of any one of embodiments E1-E56, E58, or E60-E123, to a tumor (e.g., a metastatic tumor) or a lesion (e.g., a skin lesion) of a subject.
E130. A method for preventing cancer relapse comprising contacting (e.g., administering) a microneedle device, or a plurality of microneedles, of any one of embodiments E1-E56, E58, or E60-E123, to a tumor (e.g., a metastatic tumor, e.g., on the skin) of a subject.
E131. A method for treating a cancer (e.g., a metastatic cancer) comprising contacting (e.g., administering) a microneedle device, or a plurality of microneedles, of any one of embodiments E1-E56, E58, or E60-E123, to a location of surgical resection, or a location proximal thereto, of a subject.
E132. A method for treating a cancer (e.g., a metastatic cancer) comprising contacting (e.g., administering) a microneedle device, or a plurality of microneedles, of any one of embodiments E1-E56, E58, or E60-E123, to a location of a tumor (e.g., a metastatic tumor) of a subject, thereby inducing:

(i) a local immune response and/or a local cytotoxicity to the cancer (e.g., as confirmed by the death of cancer cells at, or proximal to, the location of the applied microneedle device, e.g., a reduction in local tumor size and/or local tumor burden); and/or

(ii) a distal immune response and/or a distal cytotoxicity to the cancer (e.g., as confirmed by death of cancer cells at a location distal to the location of the applied microneedle patch, e.g., a reduction in distal tumor size and/or overall tumor burden).

E133. The method of any one of embodiments E124-E132, wherein the contacting (e.g., administering) results in one or more of:

(i) an inhibition of tumor growth at or near the site of administration;

(ii) an induction of a local immune response to ablate tumors at or near the site of administration;

(iii) an increase in activated immune effector cells (e.g., T cells) in the tumor microenvironment;

(iv) a reduction in local immunosuppressive cells (e.g., regulatory T cells (Tregs));

(v) an induction of a systemic immune response to ablate tumors at distant sites;

(vi) an immunological memory to the cancer or precancerous condition;

(vii) an immune response to a tumor antigen, such as a neoantigen; and/or

(viii) prevention and/or inhibition of cancer recurrence (e.g., cancer relapse).

E134. The method of embodiment E133, wherein the immunological memory results in the ablation of a recurrent tumor at or near the site of administration and/or ablation of a recurrent tumor at a distant site, optionally when the recurrent tumor appears within about 1 to about 6 months after administration, optionally when the recurrent tumor appears within about 1 to about 5 years after administration.
E135. The method of embodiment E133 or E134, wherein the immunological memory prevents tumor recurrence.
E136. The method of any one of embodiments E133-E135, wherein the immunological memory prevents cancer recurrence within the first 5 years after initial administration of microneedle.
E137. The method of any one of embodiments E124-E136, wherein the cancer is a metastatic cancer.
E138. The method of any one of embodiments E124-E137, wherein the cancer is a relapsed cancer.
E139. The method of any one of embodiments E124-E138, wherein the cancer is a refractory cancer.
E140. The method of any one of embodiments E124-E139, wherein the cancer is chosen from an anal cancer; a basal cell carcinoma; a bladder cancer; a bone cancer; a brain tumor; a breast cancer; a cervical cancer; a colon and rectal cancer; a endometrial cancer; an esophageal cancer; a gastrointestinal stromal tumor; a gestational trophoblastic disease; a head and neck cancer; a Hodgkin lymphoma; a Kaposi sarcoma; a kidney (renal cell) cancer; a leukemia; a liver cancer; a lung cancer; a malignant mesothelioma; a melanoma; a Merkel cell carcinoma; a multicentric Castleman disease; a multiple myeloma and other plasma cell neoplasms; a myeloproliferative neoplasm; a neuroblastoma; a Non-Hodgkin lymphoma; an ovarian, fallopian tube, or primary peritoneal cancer; a pancreatic cancer; a penile cancer; a pheochromocytoma and paraganglioma; a prostate cancer; a retinoblastoma; a rhabdomyosarcoma; a skin cancer; a squamous cell carcinoma; a soft tissue sarcoma; a solid tumor anywhere in the body; a stomach (gastric) cancer; a testicular cancer; a thyroid cancer; a vaginal cancer; a vulvar cancer; and a Wilms tumor and other childhood kidney cancers.
E141. The method of any one of embodiments E124-E140, wherein the cancer is a melanoma.
E142. The method of any one of embodiments E124-E140, wherein the cancer is a basal cell carcinoma.
E143. The method of any one of embodiments E124-E140, wherein the cancer is a squamous cell carcinoma.
E144. The method of any one of embodiments E124-E140, wherein the cancer is a Merkel cell carcinoma.
E145. The method of any one of embodiments E124-E140, wherein the cancer is a breast cancer.
E146. The method of any one of embodiments E124-E140, wherein the cancer is associated with a skin lesion and/or a tumor.
E147. The method of any one of embodiments E124-E140, wherein the tumor is a metastatic tumor.
E148. The method of any one of embodiments E124-E140, wherein the tumor is accessible without surgery.
E149. The method of any one of embodiments E124-E140, wherein the tumor is accessible by surgery.
E150. The method of any one of embodiments E124-E140, wherein the tumor is on the skin.
E151. The method of any one of embodiments E124-E140, wherein the tumor is on the eye.
E152. The method of any one of embodiments E129 or E133-E151, wherein the precancerous condition is a precancerous skin condition, optionally chosen from actinic keratosis (AK), lentigo maligna, leukoplakia, and Bowen's Disease.
E153. The method of any one of embodiments E124-E152, wherein the contacting (e.g., administering) occurs intratumorally.
E154. The method of any one of embodiments E124-E152, wherein the contacting (e.g., administering) occurs peritumorally.
E155. The method of any one of embodiments E124-E152, wherein the contacting (e.g., administering) occurs prior to surgical resection.
E156. The method of any one of embodiments E124-E152, wherein the contacting (e.g., administering) occurs after surgical resection.
E157. The method of any one of embodiments E124-E152, wherein the contacting (e.g., administering) occurs concurrently with surgery and/or the collection of a biopsy.
E158. The method of any one of embodiments E124-E152, wherein the contacting (e.g., administering) occurs in combination with a standard of care treatment (e.g., for a cancer or a precancerous condition), optionally chosen from surgery, chemotherapy, immunotherapy, targeted therapy, hormone therapy, and/or radiation therapy.
E159. The method of embodiment E158, wherein the standard of care treatment is administered prior to, after, or concurrently with the microneedle device.
E160. The method of any one embodiments E124-E159, wherein the subject is a human subject.
E161. A method of producing a microneedle device, the method comprising:

providing a mold including a mold body with an array of needle cavities having a predefined shape, e.g., pyramid-shaped and/or conical-shaped needle cavities, formed therein;

filling tips of the needle cavities with a composition comprising a silk fibroin, an anti-cancer agent, and/or an immunomodulatory agent solution;

drying the filled tips of the needle cavities to create silk fibroin tips, and optionally annealing the needle tips;

filling the needle cavities of the mold with a base (e.g., dissolvable base) solution;

drying the base solution to create base layers for the silk fibroin tips; and

(optionally) applying a backing to the base layers to create a microneedle device.

E162. A method of producing a microneedle device, the method comprising:

providing a mold including a mold body with an array of needle cavities having a predefined shape, e.g., pyramid-shaped and/or conical-shaped needle cavities, formed therein;

filling tips of the needle cavities with a composition comprising a silk fibroin, and a therapeutic agent (e.g., an anti-cancer agent, an immunomodulatory agent, or both) solution;

drying the filled tips of the needle cavities to create silk fibroin tips, and optionally annealing the silk fibroin tips;

further filling the needle cavities of the mold with a first base (e.g., dissolvable base) solution;

drying the first base solution to form a first base layer;

(optionally) forming one or more additional base layers by adding one or more additional base solutions to the first base layer, and drying the additional base solutions, optionally, wherein the additional base solutions are different to the first base solution, and

(optionally) applying a backing to the base layer (e.g., the first base layer, or the one or more additional base layers), thereby producing a microneedle device.

E163. The method of embodiment E161 or E162, wherein the base solution (e.g., the first base solution, the one or more additional base solutions, or both) comprise a molten liquid.
E164. The method of embodiment E161 or E162, wherein the base solution (e.g., the first base solution, the one or more additional base solutions, or both) comprise a slurry.
E165. The method of any one of embodiments E161-E163, wherein filling (e.g., the mold or needle cavities) comprises filling with a base solution comprising a molten liquid.
E166. The method of any one of embodiments E161-E165, wherein filling (e.g., the mold or needle cavities) comprises filling with a base solution comprising a slurry.
E167. The method of any one of embodiments E161-E166, further comprising solidifying the base layer (e.g., the first base layer, the one or more additional base layers, or both) using a chemical reaction (e.g., after filling).
E168. The method of any one of embodiments E161-E167, further comprising removing the microneedle device from the mold, optionally before applying the backing.
E169. The method of any one of embodiments E161-E168, wherein the microneedle device is removed by bending the mold away from the microneedle device.
E170. The method of any one of embodiments E161-E169, further comprising packaging microneedle devices in a container with low moisture vapor transmission rate with a desiccant to maintain between about 0% and about 50% (e.g., between about 0% and 10%, between about 10% and about 20%, between about 20% and about 30%, between about 30% and about 40%, or between about 40% and 50%, e.g., about 25%) relative humidity inside the package.
E171. The method of any one of embodiments E161-E170, wherein the silk fibroin, anti-cancer agent, and/or immunomodulatory agent solution is dispensed into each needle cavity in the mold via nanoliter printing.
E172. The method of any one of embodiments E161-E171, wherein filling the tips of the needle cavities includes dispensing a solution, e.g., a silk fibroin, an anti-cancer agent, and/or an immunomodulatory agent solution, into each needle cavity.
E173. The method of any one of embodiments E161-E172, wherein drying the filled tips of the needle cavities includes a primary drying step and a secondary drying step.
E174. The method of any one of embodiments E161-E173, wherein drying the base (e.g., dissolvable base) solution includes subjecting the mold to a centrifuge at 3900 rpm for 2 minutes and topping off the needle cavities with 50 μL of base solution.
E175. The method of any one of embodiments E161-E174, wherein the base filling occurs by nanoliter (nL) dispensing (e.g., nanoliter printing).
E176. The method of any one of embodiments E161-E175, further comprising an annealing step (e.g., before filling the base) after the filling the tips of the needle cavities.
E177. The method of any one of embodiments E161-E176, further comprising a water annealing step (e.g., before filling the base) after the filling the tips of the needle cavities.
E178. The method of any one of embodiments E161-E177, wherein the backing layer includes one of a paper backing layer and an adhesive plastic tape.
E179. The method of any one of embodiments E161-E178, wherein the backing layer comprises an adhesive coated plastic tape.
E180. The method of any one of embodiments E161-E179, wherein the backing layer comprises a porous layer.
E181. The method of any one of embodiments E161-E180, wherein the backing layer comprises one or more adhesives selected from the group consisting of acrylic, acrylate, cyanoacrylate, silicone, polyurethane, and synthetic rubber.
E182. The method of any one of embodiments E161-E181, wherein the backing layer comprises an adhesive capable of curing by light irradiation.
E183. The microneedle device, the plurality of microneedles, the microneedle, or method of any one of embodiments E1-E182, wherein the microneedle device, the plurality of microneedles, the microneedle, or a component thereof, comprises silk fibroin in an amount of about 0.5 μg to about 500 μg silk fibroin (e.g., about 0.5 μg to about 5 μg, or about 1 μg to about 10 μg, or about 5 μg to about 15 μg, or about 10 μg to about 20 μg, or about 15 μg to about 25 μg, or about 20 μg to about 30 μg, or about 25 μg to about 35 μg, or about 30 μg to about 40 μg, or about 35 μg to about 45 μg, or about 40 μg to about 50 μg, or about 45 μg to about 55 μg, or about 50 μg to about 60 μg, or about 55 μg to about 65 μg, or about 60 μg to about 70 μg, or about 65 μg to about 75 μg, or about 70 μg to about 80 μg, or about 75 μg to about 85 μg, or about 80 μg to about 90 μg, or about 85 μg to about 95 μg, or about 90 μg to about 100 μg, or about 95 μg to about 150 μg, or about 125 μg to about 175 μg, or about 150 μg to about 200 μg, or about 225 μg to about 275 μg, or about 250 μg to about 300 μg, or about 325 μg to about 375 μg, or about 350 μg to about 400 μg, or about 425 μg to about 475 μg, or about 450 μg to about 500 μg silk fibroin).
E184. The microneedle device, the plurality of microneedles, the microneedle, or method of any one of embodiments E1-E183, wherein the microneedle device, the plurality of microneedles, the microneedle, or a component thereof, comprises silk fibroin in an amount of about 1% to about 75% by weight, of silk fibroin (e.g., about 1, 2, 3, 4, 5, 6, 7, 8, 9, 10, 15, 20, 25, 30, 35, 40, 45, 50, 55, 60, 65, 70, or 75% by weight, of silk fibroin).

### BRIEF DESCRIPTION OF THE DRAWINGS

FIG. 1 is a schematic drawing of the microneedle fabrication process in accordance with an example of the present disclosure.

FIG. 2 is a series of microneedles configured to release different therapeutic agents by sustained release or by burst (referred to as “bolus”) release in accordance with an example of the present disclosure. The left most microneedle if configured to release an anti-cancer agent, such as a chemotherapeutic agent, over a two-week period. The middle microneedle is configured to release an immunomodulatory agent, such as a checkpoint inhibitor (e.g., an anti-PD1 antibody) over a two-week period. The right most microneedle is configured to release an immunomodulatory agent, such as a cytokine (e.g., IL-2) rapidly over a short period of time, e.g., minutes.

FIG. 3 illustrates a completed microneedle device having an array of microneedles applied to a backing or “handle” layer in accordance with an example of the present disclosure.

FIG. 4 illustrates a microneedle device in accordance with an example of the present disclosure. The microneedle device comprises a plurality of microneedles having sufficient mechanical properties (e.g., strength) and suitable geometry (e.g. tip sharpness, tip included angle, length, and inter-needle spacing) to pierce a biological barrier (e.g., skin) to achieve a local and/or a systemic delivery of a therapeutic agent or a combination of therapeutic agents (e.g., an anti-cancer agent, an immunomodulatory agent, or a combination thereof) to a subject.

FIG. 5 illustrates various molecular weight profiles of silk fibroin solutions useful in fabricating a microneedle described herein.

FIG. 6A illustrates a dosage regimen used for comparing bolus intratumoral (IT) doses to daily IT doses of IL-2 or gemcitabine in an exemplary mouse model; FIG. 6B is a graph depicting the tumor volume over time in mice treated with either bolus IL-2 (IT) or daily IL-2 (IT); FIG. 6C is a graph comparing survival in mice treated with either bolus (IT) or daily IL-2 (IT); FIG. 6D is a graph depicting the tumor volume over time in mice treated with either bolus gemcitabine (IT) or daily gemcitabine (IT); FIG. 6E is a graph depicting survival in mice treated with either bolus (IT) or daily gemcitabine (IT).

FIGS. 7A-7B illustrate dosage regimens used for comparing bolus intratumoral (IT) gemcitabine (FIG. 7A) to daily IT gemcitabine (FIG. 7B) in an exemplary mouse model; FIG. 7C is a graph depicting the tumor volume over time in mice treated with either bolus (IT) or daily (IT) gemcitabine; FIG. 7D is a graph depicting the survival in mice treated with either bolus (IT) or daily (IT) gemcitabine.

FIG. 8 is a graph depicting the stability of IL-2 in an exemplary silk formulation over a period of 14 days at 4° C., room temperature (RT), or 37° C., as determined by IL-2 recovery (%).

### DETAILED DESCRIPTION

### Definitions

Unless defined otherwise, all technical and scientific terms used herein have the same meaning as commonly understood by one of ordinary skill in the art to which the present disclosure pertains.

The articles “a” and “an” are used herein to refer to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate, e.g., to perform the disclosed methods.

By “a combination” or “in combination with,” it is not intended to imply that the therapy or the therapeutic agents may be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

The term “inhibition,” “inhibitor,” or “antagonist” includes a reduction in a certain parameter, e.g., an activity, of a given molecule, e.g., an immune checkpoint inhibitor. For example, inhibition of an activity, e.g., a PD-1 or PD-L1 activity, of at least 5%, 10%, 15%, 20%, 25%, 30%, 35%, 40%, 45%, 50%, 55%, 60%, 65%, 70%, 75%, 80%, 85%, 90%, 95%, or more is included by this term. Thus, inhibition need not be 100%.

The term “activation,” “activator,” or “agonist” includes an increase in a certain parameter, e.g., an activity, of a given molecule, e.g., a costimulatory molecule. For example, increase of an activity, e.g., a costimulatory activity, of at least 5%, 10%, 15%, 20%, 25%, 30%, 35%, 40%, 45%, 50%, 55%, 60%, 65%, 70%, 75%, 80%, 85%, 90%, 95%, or more is included by this term.

The terms “anti-tumor effect” and “anti-cancer effect” are used interchangeably and refer to a biological effect which can be manifested by various means, including but not limited to, for example, a decrease in tumor volume or cancer volume, a decrease in the number of tumor cells or cancer cells, a decrease in the number of metastases, an increase in life expectancy, a decrease in tumor cell proliferation or cancer cell proliferation, a decrease in tumor cell survival or cancer cell survival, a prevention of relapse, or amelioration of various physiological symptoms associated with the cancerous condition. An “anti-tumor effect” or “anti-cancer effect” can also be manifested by the ability of the microneedles of the present disclosure in prevention of the occurrence of tumor or cancer in the first place, and/or cancer recurrence or relapse. An “anti-tumor effect” or “anti-cancer effect” can also be manifested by the ability of the microneedles of the present disclosure in the formation of an immune response to the cancer, e.g., an immune response to a cancer-associated antigen (e.g., neoantigen).

As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response. In some embodiments, a tumor antigen refers to a neoantigen (e.g., an antigen, such as a peptide, that arise from somatic mutations in a tumor that differ from wild-type antigens and which can be specific to each tumor and/or subject)

As used herein, the term “anti-cancer agent” refers to a therapy and/or drug that can induce an anti-tumor and/or anti-cancer effect. In some embodiments, the anti-cancer effect includes but is not limited to, a decrease in tumor volume or cancer volume, a decrease in the number of tumor cells or cancer cells, a decrease in the number of metastases, an increase in life expectancy, a decrease in tumor cell proliferation or cancer cell proliferation, a decrease in tumor cell survival or cancer cell survival, a prevention of relapse, or amelioration of various physiological symptoms associated with the cancerous condition.

As used herein, an “increase” or “decrease” in a measurement, unless otherwise specified, is typically in comparison to a baseline value. For example, an increase or decrease in a measurement may be in comparison to a baseline level of the measurement expected for healthy subjects. Alternatively, an increase or decrease in a measurement may be in comparison to a previous time point for the same subject, for example, prior to treatment. In some instances, an increase or decrease in a measurement may be in comparison to a previous time point for the same subject, for example, during the course of treatment.

As used herein, the term “immunomodulatory agent” refers to a therapy and/or drug that can modulate (e.g., increase and/or decrease), enhance, induce, stimulate, suppress, reduce, or up-regulate one or more aspects of an immune response, e.g., in a subject having cancer. For example, an immunomodulatory agent can enhance or promote an immune attack of a target cell, such as a cancer cell and/or can promote killing or the inhibition of growth or proliferation of a target cell, such as a cancer cell, by an immune effector cell. In some embodiments, an immunomodulatory agent administered as described herein, e.g., by a microneedle or device described herein, can enhance a subject's immune response to a cancer.

As used herein, the term “cancer” is meant to include all types of cancerous growths or oncogenic processes, metastatic tissues or malignantly transformed cells, tissues, or organs, irrespective of histopathologic type or stage of invasiveness. Cancer cells can spread locally or through the bloodstream and lymphatic system to other parts of the body. Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, gastrointestinal (e.g., colon), genitourinary tract (e.g., renal, urothelial cells), prostate, and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies such as those affecting the lung, esophagus, skin, head and neck region, oral cavity, anus, and cervix. Metastatic lesions of the aforementioned cancers can also be treated or prevented using the methods and microneedles of the present disclosure.

The terms “tumor” and “cancer” are used interchangeably herein, for example, both terms encompass solid and liquid, for example, diffuse or circulating, tumors. As used herein, the term “cancer” or “tumor” includes premalignant, as well as malignant cancers and tumors.

The term “antigen presenting cell” or “APC” refers to an immune system cell such as an accessory cell (e.g., a B-cell, a dendritic cell, and the like) that displays a foreign antigen complexed with major histocompatibility complexes (MHCs) on its surface. T-cells may recognize these complexes using their T-cell receptors (TCRs). APCs process antigens and present them to T-cells.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” or “effector cell” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector” or “effector” “function” or “response,” as that term is used herein, refers to function or response, e.g., of an immune effector cell, that enhances or promotes an immune attack of a target cell. For example, an immune effector function or response refers to a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

As used herein, the terms “treat”, “treatment” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

In the context of a nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 75%, 80%, 85%, 90%, 91%, 92%, 93%, 94%, 95%, 96%, 97%, 98% or 99% identity to a reference sequence, for example, a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term “cytokine” (for example, GM-CSF, IL-1α, IL-10, IL-2, IL-3, IL-4, IL-5, IL-6, IL-7, IL-8, IL-10, IL-12, IL-15, IL-18, IL-21, IFN-α, IFN-β, IFN-γ, MIP-1α, MIP-1β, TGF-β, TNF-α, and TNFβ) includes full length, a fragment or a variant, for example, a functional variant, of a naturally-occurring cytokine (including fragments and functional variants thereof having at least 10%, 30%, 50%, or 80% of the activity, e.g., the immunomodulatory activity, of the naturally-occurring cytokine). In some embodiments, the cytokine has an amino acid sequence that is substantially identical (e.g., at least about 85%, 90%, 91%, 92%, 93%, 94%, 95%, 96%, 97%, 98% or 99% identity) to a naturally-occurring cytokine, or is encoded by a nucleotide sequence that is substantially identical (e.g., at least about 75%, 80%, 85%, 90%, 91%, 92%, 93%, 94%, 95%, 96%, 97%, 98% or 99% identity) to a naturally-occurring nucleotide sequence encoding a cytokine. In some embodiments, as understood in context, the cytokine further comprises a receptor domain, e.g., a cytokine receptor domain (e.g., an IL-15/IL-15R).

The term “effective amount” or “therapeutically effective amount” are used interchangeably herein, and refer to an amount of a compound, formulation, material, or composition, as described herein effective to achieve a particular biological result.

The term “therapeutic” as used herein means a treatment. A therapeutic effect is obtained by reduction, suppression, remission, or eradication of a disease state (e.g., a cancer).

The term “prophylaxis” as used herein means the prevention of or protective treatment for a disease or disease state (e.g., a cancer).

“Refractory” as used herein refers to a disease, for example, cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer. “Relapsed” or “relapse” as used herein refers to the return or reappearance of a disease (for example, cancer) or the signs and symptoms of a disease such as cancer after a period of improvement or responsiveness, for example, after prior treatment of a therapy, for example, cancer therapy. The initial period of responsiveness may involve the level of cancer cells falling below a certain threshold, for example, below 20%, 15%, 10%, 5%, 4%, 3%, 2%, or 1%. The reappearance may involve the level of cancer cells rising above a certain threshold, for example, above 20%, 15%, 10%, 5%, 4%, 3%, 2%, or 1%. For example, in the context of some cancers, the reappearance may involve, for example, a reappearance of a tumor after a response. In some embodiments, a response (for example, a complete response or a partial response) can involve the absence of a detectable tumor or detectable MRD (minimal residual disease). In some embodiments, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

Ranges: throughout this disclosure, various embodiments of the invention can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

As used herein, “adjuvant” in some embodiments related to cancer treatment an adjuvant is an additional cancer treatment given after the primary treatment to lower the risk that the cancer will come back. Adjuvant therapy may include, for example, chemotherapy, radiation therapy, hormone therapy, targeted therapy, or biological therapy.

As used herein, an “adjuvant” in some embodiments related to vaccine delivery an adjuvant is a substance that is able to favor or amplify the cascade of immunological events, ultimately leading to an increased immunological response, e.g., the integrated bodily response to an antigen, including cellular and/or humoral immune responses. Non-limiting examples of adjuvants include: aluminum (e.g., aluminum gels and/or aluminum salts, such as aluminum hydroxide, aluminum phosphate, and aluminum potassium sulfate), lipids (e.g., squalene, monophosphoryl lipid A (MPL)), AS03 (e.g., an adjuvant comprising D,L-alpha-tocopherol (vitamin E), squalene, and polysorbate 80), AS04 (e.g., an adjuvant comprising a combination of aluminum hydroxide and MPL), and MF59® (e.g., an adjuvant comprising squalene).

As used herein, the term “backing” refers to a material that is suitable for bonding to and/or adhering to a component of a microneedle. In some embodiments, a backing material is suitable for bonding to and/or adhering to the dissolvable base of a microneedle described herein.

As used herein, the term “base” refers to the layer that forms the base of the microneedles (e.g., functions as the support for the distal silk tips that are loaded with an anti-cancer agent, an immunomodulatory agent, or a combination thereof), and/or can also serve as a layer connecting adjacent microneedles to form a continuous microneedle array or microneedle patch. In some embodiments, at least 10%, 20%, 30%, 40%, 50%, 60%, 70%, 80%, 90% or more of the base is dissolved after application to a biological barrier, e.g., skin, tumor, tissue, cell membrane, mucous surface, oral cavity, or buccal cavity.

As used herein, the term “dose” means the amount of an anti-cancer agent and/or immunomodulatory agent which is administered (e.g., in a microneedle described herein) to elicit an anti-cancer response and/or an immune response (e.g., a humoral and/or a cellular immune response) in an organism.

As used herein, a “standard dose” means the amount of an anti-cancer agent and/or immunomodulatory agent which is administered in a typical human dose, e.g., as approved for marketing by national or international regulatory authorities (e.g., U.S. FDA, EMEA).

As used herein, a “fractional dose” refers to a dosage comprising a portioned amount of a total dose (e.g., a standard dose) of an anti-cancer agent and/or immunomodulatory agent which is administered (e.g., in a microneedle) to elicit an anti-cancer response and/or an immune response in an organism. In some embodiments, the amount of the an anti-cancer agent and/or immunomodulatory agent which is administered in the fractional dose is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose) of the anti-cancer agent and/or immunomodulatory agent which is administered.

As used herein, the term “gelatin” refers to a water-soluble protein derived from collagen. In some embodiments, the term “gelatin” refers to a sterile nonpyrogenic protein preparation (e.g., fractions) produced by partial acid hydrolysis (type A gelatin) or by partial alkaline hydrolysis (type B gelatin) of animal collagen, most commonly derived from cattle, pig, and fish sources. Gelatin can be obtained in varying molecular weight ranges. Recombinant sources of gelatin may also be used.

As used herein, the term “polyethylene glycol (PEG)” refers to an oligomer or polymer of ethylene oxide. PEG is also known as polyethylene oxide (PEO) or polyoxyethylene (POE). The structure of PEG is commonly expressed as H—(O—CH₂—CH₂)ₙ—OH.

As used interchangeably herein, the terms “sustained-release silk fibroin tip” refers to the distal end, e.g., tip, of a microneedle capable of piercing a biological barrier, e.g., the skin, mucous surface, tumor, oral cavity, or buccal cavity, of a subject and being deposited within the biological barrier, a skin layer (e.g., the dermis). In embodiments, the tip comprises a silk fibroin protein in an amount sufficient to sustain the release of a therapeutic agent, such as an anti-cancer agent and/or immunomodulatory agent, for a prolonged period of time, e.g., for at least about 1 day (e.g., about 1, 2, 3, 4, 5, 6, 7, 8, 9, 10, 11, 12, 13, or 14 or more days, e.g., between about 4 days and about 30 days, e.g., between about 1-2 weeks, about 1-3 weeks, or about 1-4 weeks, e.g., about 2-12 months).

As used herein, the term “microneedle” refers to a structure having at least two, more typically, three components, e.g., layers, for transport or delivery of a therapeutic agent, such as an anti-cancer agent, an immunomodulatory agent, or a combination thereof, across a biological barrier, such as the skin, tissue, tumor, or cell membrane. In some embodiments, a microneedle comprises a base (e.g., a dissolvable base as described herein), a tip (e.g., an implantable tip as described herein), and optionally, a backing material. In embodiments, a microneedle has dimension of between about 350 μm to about 1500 μm in height (e.g., between about 350 μm to about 1500 μm, e.g., about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm)). In some embodiments, the microneedle is fabricated to have any dimension and/or geometry to enable the deployment of a silk fibroin tip, e.g., an implantable sustained-release tip, at a depth between about 100 μm and about 900 μm (e.g., at a depth of about 800 μm) into the dermis layer of the skin for release, e.g., controlled- or sustained-release of an anti-cancer agent, an immunomodulatory agent, or a combination thereof.

As used herein, the terms “microneedle patch” and “microneedle array” refer to a device comprising a plurality of microneedles, e.g., silk fibroin-based microneedles, e.g., arranged in a random or predefined pattern, such as an array. In some embodiments, a microneedle patch or microneedle array of the present disclosure may include silk fibroin in an amount of about 0.5 μg to about 500 μg silk fibroin. In some embodiments, a microneedle patch or microneedle array of the present disclosure may include silk fibroin in an amount of about 0.5 μg to about 5 μg, or about 1 μg to about 10 μg, or about 5 μg to about 15 μg, or about 10 μg to about 20 μg, or about 15 μg to about 25 μg, or about 20 μg to about 30 μg, or about 25 μg to about 35 μg, or about 30 μg to about 40 μg, or about 35 μg to about 45 μg, or about 40 μg to about 50 μg, or about 45 μg to about 55 μg, or about 50 μg to about 60 μg, or about 55 μg to about 65 μg, or about 60 μg to about 70 μg, or about 65 μg to about 75 μg, or about 70 μg to about 80 μg, or about 75 μg to about 85 μg, or about 80 μg to about 90 μg, or about 85 μg to about 95 μg, or about 90 μg to about 100 μg, or about 95 μg to about 150 μg, or about 125 μg to about 175 μg, or about 150 μg to about 200 μg, or about 225 μg to about 275 μg, or about 250 μg to about 300 μg, or about 325 μg to about 375 μg, or about 350 μg to about 400 μg, or about 425 μg to about 475 μg, or about 450 μg to about 500 μg. In some embodiments, a microneedle patch or microneedle array of the present disclosure may include silk fibroin in an amount of at least about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin. In some embodiments, a microneedle patch or microneedle array of the present disclosure may include silk fibroin in an amount of at most about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin. In some embodiments, a microneedle patch or microneedle array of the present disclosure may include silk fibroin in an amount of about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin.

In some embodiments, a microneedle patch or microneedle array of the present disclosure includes about 1% to about 75%, about 1% to about 5%, about 10% to about 60%, about 15% to about 50%, or about 20% to about 40% by weight, of silk fibroin. In some embodiments, a microneedle patch or microneedle array of the present disclosure includes at least about 1, 2, 3, 4, 5, 6, 7, 8, 9, 10, 15, 20, 25, 30, 35, 40, 45, 50, 55, 60, 65, 70, or 75% by weight, of silk fibroin. In some embodiments, a microneedle patch or microneedle array of the present disclosure includes at most about 1, 2, 3, 4, 5, 6, 7, 8, 9, 10, 15, 20, 25, 30, 35, 40, 45, 50, 55, 60, 65, 70, or 75% by weight, of silk fibroin. In some embodiments, a microneedle patch or microneedle array of the present disclosure includes about 1, 2, 3, 4, 5, 6, 7, 8, 9, 10, 15, 20, 25, 30, 35, 40, 45, 50, 55, 60, 65, 70, or 75% by weight, of silk fibroin.

As used herein, the term “silk fibroin” includes silkworm fibroin and insect or spider silk protein. Any type of silk fibroin can be used according to various aspects described herein. Silk fibroin produced by silkworms, such as Bombyx mori, is the most common and represents an earth-friendly, renewable resource. For instance, silk fibroin used in a microneedle (e.g., a silk fibroin tip, e.g., an implantable controlled- or sustained-release tip of a microneedle) may be obtained by removing sericin from the cocoons of B. mori. In some embodiments, the silk fibroin is a regenerated silk fibroin, e.g., a silk fibroin obtained after extraction of sericin from the cocoons of B. mori, and an additional processing e.g. via a boiling step. Organic silkworm cocoons are also commercially available. There are many different silks, however, including spider silk (e.g., obtained from Nephila clavipes), transgenic silks, recombinant and/or genetically engineered silks, such as silks from bacteria, yeast, mammalian cells, transgenic animals, or transgenic plants (see, e.g., WO 97/08315; U.S. Pat. No. 5,245,012), and variants thereof, that can be used.

As used herein, a “subject” refers to a human or animal. Usually the animal is a vertebrate such as a primate, rodent, domestic animal or game animal. Primates include chimpanzees, cynomolgus monkeys, spider monkeys, and macaques (e.g., Rhesus). Rodents include mice, rats, woodchucks, ferrets, rabbits and hamsters. Domestic and game animals include cows, horses, pigs, deer, bison, buffalo, feline species (e.g., domestic cat), canine species (e.g., dog, fox, wolf), avian species (e.g., chicken, emu, ostrich), and fish (e.g., trout, catfish and salmon). In certain embodiments of the aspects described herein, the subject is a mammal (e.g., a primate, e.g., a human). A subject can be male or female. In certain embodiments, the subject is a mammal. The mammal can be a human, non-human primate, mouse, rat, dog, cat, horse, or cow, but are not limited to these examples. In addition, the methods and formulations described herein can be used to treat domesticated animals and/or pets. In some embodiments, the term “subject” is intended to include living organisms in which an immune response can be elicited (for example, mammals, for example, human).

As used herein, the term “release” and “controlled- or sustained-release” refers to the release of a therapeutic agent (e.g., from a microneedle, microneedle device, formulation, composition, article, device, or preparation described herein, e.g., from a silk fibroin-based microneedle tip as described herein), such as an anti-cancer agent, an immunomodulatory agent, or a combination thereof, over a period of time, e.g., for at least about 1 to about 28 days (e.g., about 1, 2, 3, 4, 5, 6, 7, 8, 9, 10, 11, 12, 13, 14, 15, 16, 17, 18, 19, 20, 21, 22, 23, 24, 25, 26, 27, or 28 or more days, e.g., between about 4 days and about 14 days, e.g., between about 1-2 weeks, about 1-3 weeks, or about 1-4 weeks, e.g., between about 1 month to about 3 months). In some embodiments, the controlled- or sustained-release of a therapeutic agent, such as an anti-cancer agent, an immunomodulatory agent, or a combination thereof, over a time period of about 1 to about 14 days, e.g., about 1, 2, 3, 4, 5, 6, 7, 8, 9, 10, 11, 12, 13, or 14 days, by a microneedle, microneedle device, formulation, composition, article, device, or preparation as described herein can result, e.g., in an anti-cancer response and/or immune response in a subject. In some embodiments, the controlled- or sustained-release of a therapeutic agent, such as an anti-cancer agent, an immunomodulatory agent, or a combination thereof, over a time period of about 1 to about 4 weeks, e.g., about 1, 2, 3, or 4 weeks, by a microneedle, microneedle device, formulation, composition, article, device, or preparation as described herein can result, e.g., in an anti-cancer response and/or immune response in a subject. In some embodiments, the formulations and preparations comprising silk fibroin and a therapeutic agent, such as an anti-cancer agent, an immunomodulatory agent, or a combination thereof, have controlled- or sustained-release properties (e.g., are formulated and/or configured to release a therapeutic agent, e.g., into the skin of the subject, over a period of, or at least 1, 5, 10, 15, 30, 45 minutes; a period of, or at least, 1, 2, 3, 4, 5, 10, 24 hours; a period of, or at least, 1, 2, 3, 4, 5, 6, 7, 8, 9, 10, 11, 12, 13, 14 days; a period of, or at least, 1, 2, 3, 4, 5, 6, 7, 8 weeks; a period of, or at least 1, 2, 3, 4, 5, 6, 7, 8, 9, 10, 11 months; a period of, or at least, 1, 2, 3, 4, 5 years, or longer. In some embodiments, the controlled- or sustained release is by burst release. In some embodiments, the microneedles and microneedle devices described herein can be configured for the sustained release of an anti-cancer agent, such as a chemotherapeutic agent, of about 28 days or more.

As used herein, the terms “therapeutic agent” and “active agent” are art-recognized terms and refer to any chemical moiety that is a biologically, physiologically, or pharmacologically active substance that acts locally or systemically in a subject. Various forms of a therapeutic agent may be used which are capable of being released from the microneedles described herein into adjacent tissues or fluids upon administration to a subject.

Various embodiments of the compositions and methods herein are described in further detail below. Additional definitions are set out throughout the specification.

### Description

Provided herein are silk fibroin-based microneedles, and silk fibroin-based microneedle devices (e.g., microneedle patches), configured to incorporate, and subsequently release (e.g., administer), an effective amount of a therapeutic agent (e.g., an anti-cancer agent, an immunomodulatory agent, or a combination thereof) into a subject (e.g., into and/or across a biological barrier, such as the skin, of a subject). Use of the silk fibroin-based microneedles, and silk fibroin-based microneedle devices (e.g., microneedle patches), can result in an anti-cancer effect and/or an immunity (e.g., a prolonged, broad spectrum immunity to a cancer associated antigen, e.g., neoantigen) to a cancer in a patient.

Without wishing to be bound by theory, the administration of a microneedle or a microneedle device disclosed herein to an application site (e.g. a site of abnormal cells, e.g., a tumor or a lesion) on a subject results in the release of an effective amount of a therapeutic agent (e.g., an anti-cancer agent, an immunomodulatory agent, or a combination thereof) into the subject, thereby inducing a local treatment effect (e.g., a local anti-cancer effect) at or near the site of administration. In addition to this local treatment effect, the administration of a microneedle or a microneedle device disclosed herein can also result in a systemic treatment effect (e.g., a systemic anti-cancer effect) at distant sites having similar features as the application site (e.g., a distant site of abnormal cells, e.g., a distant tumor or a distant lesion).

In some embodiments, the microneedle or microneedle device disclosed herein may be administered to a subject to effect the sustained release of an effective amount of a therapeutic agent (e.g., an anti-cancer agent, an immunomodulatory agent, or a combination thereof) into the subject over a pre-defined period of time, e.g., 4-15 days. In some embodiments, the microneedle or microneedle device disclosed herein may be administered to a subject to effect the sustained release of an effective amount of a therapeutic agent (e.g., an anti-cancer agent, an immunomodulatory agent, or a combination thereof) into the subject over a pre-defined period of time, e.g., at least 1, 5, 10, 15, 30, 45 minutes; a period of, or at least, 1, 2, 3, 4, 5, 10, 24 hours; a period of, or at least, 1, 2, 3, 4, 5, 6, 7, 8, 9, 10, 11, 12, 13, 14 days; a period of, or at least, 1, 2, 3, 4, 5, 6, 7, 8 weeks; a period of, or at least 1, 2, 3, 4, 5, 6, 7, 8, 9, 10, 11 months; or a period of, or at least, 1, 2, 3, 4, 5 years, or longer.

In some embodiments, the microneedle or microneedle device disclosed herein may be administered to a subject to effect the burst release of an effective amount of a therapeutic agent (e.g., an anti-cancer agent, an immunomodulatory agent, or a combination thereof) into the subject over a pre-defined period of time, e.g., about 24 hour or less. In some embodiments, the microneedle or microneedle device disclosed herein may be administered to a subject to effect the burst release of an effective amount of a therapeutic agent (e.g., an anti-cancer agent, an immunomodulatory agent, or a combination thereof) into the subject over a pre-defined period of time, e.g., at least 1, 5, 10, 15, 30, 45 minutes; or a period of, or at least, 1, 2, 3, 4, 5, 10, 24 hours.

In some embodiments, the microneedle or microneedle device disclosed herein may be administered in combination with a second therapeutic agent or procedure.

Accordingly, disclosed herein are manufacturing process of making improved silk fibroin-based microneedles and devices comprising the same for use in treating a disease in a subject.

### Silk Fibroin-Based Microneedles

In some embodiments, the present disclosure provides silk fibroin-based microneedles and microneedle devices (e.g., microneedle patches) for the transport and release of an effective amount of a therapeutic agent, such as an anti-cancer agent, an immunomodulatory agent, or a combination thereof, into and/or across a biological barrier (e.g., skin, tumor, mucosa, tissue, such as organ tissue and muscle tissue, buccal cavity, oral cavity, or a cell membrane).

Accordingly, the silk fibroin-based microneedles and microneedle devices disclosed herein can be configured to have various mechanical properties (e.g., strength), designs and geometries (e.g., needle shape and sharpness), and release kinetics (e.g., sustained release and/or burst release) to enable the administration of an effective amount of a therapeutic agent such as an anti-cancer agent, an immunomodulatory agent, or a combination thereof, to a subject, e.g., to treat a disease or disorder, such as a cancer and/or skin condition.

### Mechanical Properties

Microneedles, including the silk fibroin-based microneedles disclosed herein, can be designed to be inserted into the skin without breaking. In some embodiments, microneedle insertion is achieved by using needles with sharp tips and with sufficient length to overcome the deflection of a biological barrier's (e.g., the skin's) surface that occurs before insertion. In some embodiments, microneedle integrity during insertion can be achieved by minimizing the required insertion force, e.g., by using sharp-tipped needles, by maximizing the mechanical strength, and/or by optimizing the needle diameter (See, e.g., Park et al. J. Korean Phys. Soc. 56(4): 1223-1227, 2010). Accordingly, in some embodiments, the mechanical properties of the silk fibroin-based microneedles are optimized (e.g., by adjusting the concentration of various formulation components, including silk fibroin crystallinity, disclosed herein) to avoid sudden failure of a microneedle by buckling, and to enable successful penetration and insertion of the microneedle into the biological barrier (e.g., skin, tumor, tissue, cell membrane, mucous surface, oral cavity, or buccal cavity). In some embodiments, the microneedles disclosed herein can be configured to have geometries below a 4:1 aspect ratio of length-to-equivalent diameter and/or a to have mechanical strength characterized by Young's modulus greater than 500 MPa and failure stress greater than 10 MPa. In some embodiment, the microneedles have a 15 degree included angle and/or about a 4:1 aspect ratio. In some embodiments, the base formula has a Flex modulus of about 1000 to about 1500 MPa and a failure stress of about 15 to about 30 MPa. In some embodiments, the microneedles disclosed herein can be configured to have geometries below a 2:1 aspect ratio of length-to-equivalent diameter. In some embodiments, the microneedles have about a 5 degree to about a 50 degree included angle. For example, the microneedle can have an included angle of about 5, 10, 15, 20, 25, 30, 35, 40, 45, or 50 degrees. In some embodiments, the microneedles have a 30 degree included angle. In some embodiments, the microneedles have a 30 degree included angle and/or about a 1.87:1 aspect ratio. In some embodiments, the base formula has a Flexural modulus of about 50 to about 1500 MPa (e.g., about 50, 60, 70, 80, 90, 100, 150, 200, 250, 300, 350, 400, 450, 500, 550, 560, 750, 800, 850, 900, 950, 1000, 1100, 1200, 1300, 1400, or 1500 MPa), and/or a flexural failure stress of about 1 to about 30 MPa (e.g., about 1, 2, 3, 4, 5, 6, 7, 8, 9, 10, 15, 20, 25, 30 MPa).

### Microneedle Designs

In some embodiments, the present disclosure provides silk fibroin-based microneedles, and devices comprising the same, that have various design configurations. The silk fibroin-based microneedles disclosed herein can be in any shape and/or geometry suitable for use in piercing a biological barrier (e.g., skin, tumor, tissue, cell membrane, mucous surface, oral cavity, or buccal cavity) to enable release, e.g., sustained-release and/or burst release, of a therapeutic agent (e.g., an anti-cancer agent, an immunomodulatory agent, or a combination thereof) within a subject. Non-limiting examples of the shape and/or geometry of the microneedles include: a cylindrical shape, a wedge-shape, a cone-shape, a pyramid-shape, and/or an irregular-shape, or any combinations thereof.

In some embodiments, the silk fibroin-based microneedles are comprised of dissolving and/or degradable microneedles. In some embodiments, a dissolvable and/or degradable (e.g., resorbable) microneedle of the present disclosure encapsulates the therapeutic agent (e.g., an anti-cancer agent, an immunomodulatory agent, or a combination thereof) in a formulation, such as a silk fibroin-based formulation, which dissolves and/or degrades once inside the subject (e.g., skin). In some embodiments, the release of a therapeutic agent (e.g., an anti-cancer agent, an immunomodulatory agent, or a combination thereof) from a degradable microneedle is by protease mediated degradation. In some embodiments, only a portion (e.g., tip, e.g., silk fibroin tip) of the silk fibroin-based microneedle is configured to be dissolvable and/or degradable. In some embodiments, substantially all of the silk fibroin-based microneedle is dissolvable and/or degradable. In some embodiments, the release of a therapeutic agent (e.g., an anti-cancer agent, an immunomodulatory agent, or a combination thereof) from a dissolvable and/or degradable (e.g., resorbable) microneedle is by diffusion-controlled release through the material of the microneedle.

In some embodiments, the silk fibroin-based microneedles are solid microneedles. In some embodiments, a solid microneedle of the present disclosure is designed as a two-part system. In some embodiments, a microneedle device comprising silk fibroin-based solid microneedles is first applied to the skin to create microscopic wells just deep enough to penetrate the outermost layer of a biological barrier (e.g., skin), and then the therapeutic agent (e.g., an anti-cancer agent, an immunomodulatory agent, or a combination thereof) is applied via a transdermal patch. In some embodiments, the release of a therapeutic agent (e.g., an anti-cancer agent, an immunomodulatory agent, or a combination thereof) from a solid microneedle is by diffusion through the material of the microneedle and/or degradation of the material of the microneedle, e.g., protease mediated degradation. In some embodiments where the solid microneedles degrade, the solid microneedle are typically referred to as dissolving or resorbable microneedles.

In some embodiments, the silk fibroin-based microneedles are hollow microneedles. In some embodiments, a hollow microneedle of the present disclosure comprises a reservoir that delivers the therapeutic agent (e.g., an anti-cancer agent, an immunomodulatory agent, or a combination thereof) directly into the site of application (e.g., a biological barrier, e.g., skin).

In some embodiments, the silk fibroin-based microneedles are coated microneedles. In some embodiments, the therapeutic agent (e.g., an anti-cancer agent, an immunomodulatory agent, or a combination thereof) is applied directly to a portion (e.g., a surface) of a microneedle. In some embodiments, the coated microneedles are also coated with a surfactant (e.g., an octyl phenol ethoxylate (e.g., Triton-X), a polysorbate, a poloxamer, such as P188, and/or a polyethoxylated alcohol) and/or a thickening agent to assure that the therapeutic agent is delivered properly.

In some embodiments, a silk fibroin-based microneedle of the present disclosure can comprise the following layers: (1) a backing material (optional); (2) a base (e.g., a dissolvable base); and (3) a silk fibroin tip. For example, the microneedles described herein may include a backing material (optional) applied to a dissolvable base layer that supports a distal silk fibroin tip comprising a silk fibroin and a therapeutic agent (e.g., an anti-cancer agent, an immunomodulatory agent, or a combination thereof).

In some embodiments, a silk fibroin-based microneedle of the present disclosure may include silk fibroin in an amount of about 0.5 μg to about 500 μg silk fibroin. In some embodiments, a silk fibroin-based microneedle of the present disclosure may include silk fibroin in an amount of about 0.5 μg to about 5 μg, or about 1 μg to about 10 μg, or about 5 μg to about 15 μg, or about 10 μg to about 20 μg, or about 15 μg to about 25 μg, or about 20 μg to about 30 μg, or about 25 μg to about 35 μg, or about 30 μg to about 40 μg, or about 35 μg to about 45 μg, or about 40 μg to about 50 μg, or about 45 μg to about 55 μg, or about 50 μg to about 60 μg, or about 55 μg to about 65 μg, or about 60 μg to about 70 μg, or about 65 μg to about 75 μg, or about 70 μg to about 80 μg, or about 75 μg to about 85 μg, or about 80 μg to about 90 μg, or about 85 μg to about 95 μg, or about 90 μg to about 100 μg, or about 95 μg to about 150 μg, or about 125 μg to about 175 μg, or about 150 μg to about 200 μg, or about 225 μg to about 275 μg, or about 250 μg to about 300 μg, or about 325 μg to about 375 μg, or about 350 μg to about 400 μg, or about 425 μg to about 475 μg, or about 450 μg to about 500 μg. In some embodiments, a silk fibroin-based microneedle of the present disclosure may include silk fibroin in an amount of at least about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin. In some embodiments, a silk fibroin-based microneedle of the present disclosure may include silk fibroin in an amount of at most about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin. In some embodiments, a silk fibroin-based microneedle of the present disclosure may include silk fibroin in an amount of about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin.

In some embodiments, a silk fibroin-based microneedle of the present disclosure includes about 1% to about 75%, about 1% to about 5%, about 10% to about 60%, about 15% to about 50%, or about 20% to about 40% by weight, of silk fibroin. In some embodiments, a silk fibroin-based microneedle of the present disclosure includes at least about 1, 2, 3, 4, 5, 6, 7, 8, 9, 10, 15, 20, 25, 30, 35, 40, 45, 50, 55, 60, 65, 70, or 75% by weight, of silk fibroin. In some embodiments, a silk fibroin-based microneedle of the present disclosure includes at most about 1, 2, 3, 4, 5, 6, 7, 8, 9, 10, 15, 20, 25, 30, 35, 40, 45, 50, 55, 60, 65, 70, or 75% by weight, of silk fibroin. In some embodiments, a silk fibroin-based microneedle of the present disclosure includes about 1, 2, 3, 4, 5, 6, 7, 8, 9, 10, 15, 20, 25, 30, 35, 40, 45, 50, 55, 60, 65, 70, or 75% by weight, of silk fibroin.

### Backing

Exemplary backing materials that can be used in the fabrication of a microneedle of the present disclosure include, but are not limited to, a solid support, e.g., a paper-based material, a plastic material, a polymeric material, or a polyester-based material (e.g., a Whatman 903 paper, a polymeric tape, a plastic tape, an adhesive-backed polyester tape, or other suitable tape). In some embodiments, the backing comprises a Whatman 903 paper. In some embodiments, the backing comprises a polyester tape. In some embodiments, the polyester tape comprises an adhesive-backed polyester tape. In some embodiments, the backing material may be coated (e.g., at least on one side) with an adhesive suitable for bonding to and/or adhering to the dissolvable base of a microneedle described herein. In some embodiments, the backing may extend beyond the microneedle array. In some embodiments, the adhesive coated on the backing may be suitable for adhering to the subject's skin to hold the array in place.

The backing materials used in the microneedles of the present disclosure may have various properties, including, but not limited to, the ability to bond and/or adhere to the dissolving base layer to permit demolding. A backing material can be strong enough for the backing to maintain patch integrity, e.g., if the dissolving base layer has cracks or discontinuities. The backing material may be sufficiently flexible so as to conform, for example, to a non-flat surface, such as a skin surface. In particular, the backing can be flexible enough during wear time, such as after the patch is applied (e.g., pressed into) the skin. The backing may comprise and/or consist of a non-dissolving material, such that the backing maintains its integrity after patch application to a skin surface and during patch removal from a skin surface.

In some embodiments, the backing layer comprises an adhesive coated plastic tape, a porous material, and/or an adhesive. In some embodiment, the adhesive is selected from the group consisting of acrylic, acrylate, cyanoacrylate, silicone, polyurethane, and synthetic rubber. In some embodiments, the adhesive comprises a material that can be cured by light irradiation.

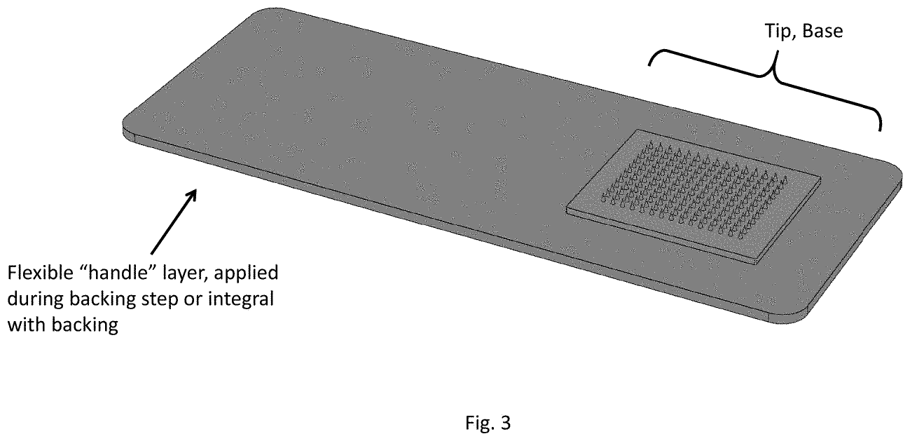

The backing may have any dimension suitable for application to a target biological barrier, e.g., a skin surface. In some embodiments, the dimensions of the backing comprises a circle. In some embodiments, the dimensions of the backing comprises a rectangle (e.g., a square, or rectangular strip). The backing may have rounded corners (e.g., a square or rectangle with rounded corners). The backing may further comprise an extension, e.g., to be used as a “handle” (see, e.g., FIG. 3).

The backing may have any suitable dimension, e.g., to accommodate the microneedle array, and/or to better suit the intended site of application. For example, the backing can have a diameter of between about 5 mm and about 50 mm, e.g., between about 6 mm and about 40 mm, between about 8 mm and about 35 mm, between about 10 mm and about 30 mm, between about 11 mm and about 25 mm, between about 12 mm and about 24 mm, between about 10 mm and about 20 mm, or between about 10 mm and about 15 mm. In some embodiments, the backing has a diameter of about 5 mm or more, e.g., about 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or more.

In some embodiments, the backing is a circle with a diameter of between about 8 mm and about 16 mm, e.g., about 8 mm, 9 mm, 10 mm, 11 mm, 12 mm, 13 mm, 14 mm, 15 mm, or 16 mm. In some embodiments, the backing is a square with dimensions of about 8 mm×8 mm, about 9 mm×9 mm, about 10 mm×10 mm, about 11 mm×11 mm, about 12 mm×12 mm, about 13 mm×13 mm, about 14 mm×14 mm, about 15 mm×15 mm, or about 16 mm×16 mm. In some embodiments, the backing is rectangular (e.g., a rectangular strip), with a width of about 8 mm or more (e.g., a width of about 9 mm, about 10 mm, about 11 mm, about 12 mm, about 13 mm, or more) and a length that is larger than the width, e.g., a length of about 10 mm or more (e.g., a length of about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, or more).

In some embodiments, the dimensions of the backing can be a 12 mm diameter circle. In some embodiments, the dimensions of the backing can be a 12 mm wide strip with a “handle” section of up to 12 mm length beyond the edge of the 12 mm×12 mm patch (see, e.g., FIG. 3). In some embodiments, a 12 mm square polyester tape with an approximately 12 mm square extended “handle” can be used (see, e.g., FIG. 3). In some embodiments, the backing can be larger, e.g., about 25 mm square, optionally with rounded corners. In some embodiments, the backing can be about a 25 mm diameter circle. In some embodiments, the area of backing that extends beyond the array can serve to hold the patch onto the skin with a biocompatible skin adhesive.

### Dissolvable Base

The base layer (e.g., dissolving base layer) forms the base of the needles (e.g., functions as the support for the distal silk fibroin tips that can be loaded with therapeutic agent (e.g., an anti-cancer agent, an immunomodulatory agent, or a combination thereof). The base layer (e.g., dissolvable base layer) can also function as a layer connecting adjacent needles to form a microneedle array or patch.

In some embodiments, the base layer (e.g., dissolving base layer) comprises a material that can dissolve into the subject, e.g., within the intended wear time (e.g., about five minutes). In some embodiments, at least about 10%, 20%, 30%, 40%, 50%, 60%, 70%, 80%, 90%, or 100% of the dissolvable base layer is dissolved after application to a biological barrier (e.g., skin) of a subject within the intended wear time (e.g., about 1 minute, about 2 minutes, about 3 minutes, about 4 minutes, about 5 minutes, about 6 minutes, about 7 minutes, about 8 minutes, about 9 minutes, or about 10 minutes or more).

The material used in the fabrication of the dissolvable base can be sufficiently strong enough to enable the microneedle to penetrate the skin, and be tough enough (e.g., not excessively brittle) to also enable demolding of the microneedle during fabrication. The dissolvable base material can be amenable to routine handling without catastrophic failure, and can retain its mechanical properties between demolding and application (e.g., not so hygroscopic that it melts due to ambient humidity). The dissolvable base layer material can be non-toxic and non-reactogenic at the doses used in a patch. In some embodiments, the dissolvable base layer comprises a water soluble component.

Non-limiting examples of materials that may be used to fabricate the base layer (e.g., dissolvable base layer) include a polysaccharide, a disaccharide, a polymer, a protein, a plasticizer, and/or a surfactant. In some embodiments, the base layer (e.g., dissolving base layer) comprises one or more (e.g., two or more, three or more, four or more, five or more, or all) of a polysaccharide (e.g., dextran); a disaccharide (e.g., sucrose, maltose, and trehalose); a polymer (e.g., methyl cellulose, polyethylene glycol (PEG), carboxymethylcellulose (CMC), polyvinylpyrrolidone (PVP), polyvinyl alcohol (PVA), hyaluronate); a protein (e.g., gelatin, fibroin); a plasticizer (e.g., glycerol, propanediol); and a surfactant (e.g., an octyl phenol ethoxylate (e.g., Triton-X), a polysorbate, a poloxamer, and/or a polyethoxylated alcohol).

The base layers disclosed herein can comprise a polysaccharide, a disaccharide, a polymer, a protein, a plasticizer, and/or a surfactant at a concentration between about 0.001% and about 75% (e.g., between about 0.001% to about 1%, e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75%). In some instances, a dried solid base can comprise a polysaccharide, a disaccharide, a polymer, a protein, a plasticizer, and/or a surfactant at a concentration of up to about 100%. In some instances, a dried solid base can comprise a surfactant at a concentration of about 0.001%.

In some embodiments, the base layer (e.g., dissolving base layer) is configured for burst release and/or sustained release of a therapeutic agent (e.g., an anti-cancer agent, an immunomodulatory agent, or a combination thereof). In some embodiments, the base layer (e.g., dissolving base layer) comprises one or more (e.g., two or more, three or more, four or more, five or more, or six or more) of gelatin, dextran, glycerol, polyethylene glycol (PEG), sucrose, trehalose, maltose, carboxymethylcellulose (CMC), polyvinylpyrrolidone (PVP), polyvinyl alcohol (PVA), hyaluronate, methyl cellulose, and/or a surfactant (e.g., an octyl phenol ethoxylate (e.g., Triton-X), a polysorbate, a poloxamer, and/or a polyethoxylated alcohol), optionally wherein the microneedle is configured for sustained release and/or burst release.

In some embodiments, the base layer (e.g., dissolving base layer) comprises one or more (e.g., two or more, three or more, or four or more) of dextran, sucrose, glycerol, and a surfactant (e.g., an octyl phenol ethoxylate (e.g., Triton-X), a polysorbate, a poloxamer, and/or a polyethoxylated alcohol), optionally configured for sustained release.

In some embodiments, the base layer (e.g., dissolving base layer) comprises polyvinyl alcohol (PVA) and sucrose, optionally configured for burst release.

In some embodiments, the base layer (e.g., dissolving base layer) comprises a dextran. In some embodiments, the dextran can have a molecular weight of between about 30 kDa to about 600 kDa. In some embodiments, the dextran has a molecular weight of about 40 kDa, about 50 kDa, about 60 kDa, about 70 kDa, about 80 kDa, about 90 kDa, about 100 kDa, about 200 kDa, about 300 kDa, about 400 kDa, about 500 kDa, or about 600 kDa. In some embodiments, a mixture of different dextrans can be used, e.g., a mixture of dextrans having various molecular weights. In some embodiments, the dextran can be obtained and/or derived from a variety of bacterial sources, including but not limited to Leuconostoc mesenteroides.

In some embodiments, the base layer (e.g., dissolving base layer) does not comprise poly(acrylic acid) (PAA). In some embodiments, a dissolvable base layer, as described herein, has improved biocompatibility, e.g., as compared to a dissolvable base layer comprising poly(acrylic acid) (PAA). In some embodiments, the dissolvable base layer material causes a reduced inflammatory response and/or reduced tissue necrosis. In some embodiments, the dissolvable base layer material is not PAA, and induces a reduced inflammatory response and/or reduced tissue necrosis compared to PAA. In some embodiments, the dissolvable base layer material has a pH similar to that of the biological barrier into which it will be dissolved, e.g., a pH of about 4.0 to about 8.0, e.g., about 4.0, about 4.5, about 5.0, about 5.5, about 6.0, about 6.5, about 7.0, about 7.5, or about 8.0. In other embodiments, the base layer comprises a silk fibroin and/or a therapeutic agent. The base layer (e.g., dissolvable base layer) can comprises less than 98% (e.g., less than about 98%, less than about 90%, less than about 80%, less than about 70%, less than about 60%, less than about 50%, less than about less 40%, less than about 30%, less than about 20%, less than about 10%, less than about 9%, less than about 8%, less than about 7%, less than about 6%, less than about 5%, less than about 4%, less than about 3%, less than about 2%, or less than about 1%) of the total amount (e.g., dose) of a therapeutic agent loaded into the microneedle and/or microneedle device.

In some embodiments, the base layer does not comprise, e.g., a detectable amount of, a silk fibroin and/or a therapeutic agent. In some embodiments, base layer is formulated to limit and/or reduce the amount of a therapeutic agent leakage (e.g., diffusion) from the silk fibroin tips into the base layer, e.g., as compared to art known base layer formulations, e.g., base layer formulations comprising PAA. In some embodiments, a limited and/or reduced amount of therapeutic agent leakage (e.g., diffusion) from the silk fibroin tips can be determined about 1 day, about 2 days, about 3 days, about 4 days, about 5 days, or about 6 days; about 1 week, about 2 weeks, or about 3 weeks; about 1 month, about 2 months, about 3 months, about 4 months, about 5 months, about 6 months, about 7 months, about 8 months, about 9 months, about 10 months, or about 11 months; or about 1 year or more after fabrication and storage (e.g., storage at about 4° C. (e.g., refrigeration), at about 25° C. (e.g., room temperature), at about 37° C. (e.g., body temperature), at about 45° C., and/or at about 50° C.), e.g., as compared to a base layer formulation comprising PAA.

In some embodiments, the dissolvable base comprises between about 10% and about 70% gelatin (e.g., hydrolyzed gelatin) (e.g., about 10%, about 20%, about 30%, about 40%, about 50%, about 60%, or about 70% gelatin).

In some embodiments, the dissolvable base comprises between about 10% and about 70% of a plasticizer, such as glycerol (e.g., about 10%, about 20%, about 30%, about 40%, about 50%, about 60%, or about 70% glycerol). In some embodiments a plasticizer is added to reduce brittleness. For example, the brittleness of a dissolvable base layer comprising a plasticizer can be reduced by about 1%, about 2%, about 4%, about 6%, about 8%, about 10%, about 15%, about 20%, about 30%, about 40%, about 50%, about 60%, about 70%, about 80%, about 90%, about 100%, about 125%, about 150%, about 200%, about 300%, or more, relative to a dissolvable base layer substantially free of the plasticizer.

In some embodiments, the dissolvable base comprises between about 0.001% and about 5% of a surfactant described herein, such as polysorbate (e.g., about 0.001% to about 1%, or about 1% to about 5% surfactant). In some embodiments a surfactant is added to aid in processing. In some embodiments, a surfactant is added as a plasticizer.

In some embodiments, the dissolvable base comprises between about 1% and about 70% polyethylene glycol (PEG) (e.g., about 1%, about 2%, about 3%, about 4%, about 5%, about 6%, about 7%, about 8%, about 9%, about 10%, about 20%, about 30%, about 40%, about 50%, about 60%, or about 70% PEG).

In some embodiments, the dissolvable base comprises between about 1% and about 35% sucrose (e.g., about 1%, about 2%, about 3%, about 4%, about 5%, about 6%, about 7%, about 8%, about 9%, about 10%, about 15%, about 20%, about 25%, about 30%, or about 35% sucrose).

In some embodiments, the dissolvable base comprises between about 1% and about 35% CMC (e.g., about 1%, about 2%, about 3%, about 4%, about 5%, about 6%, about 7%, about 8%, about 9%, about 10%, about 15%, about 20%, about 25%, about 30%, or about 35% CMC).

In some embodiments, the dissolvable base comprises between about 10% and about 70% PVP (e.g., about 10%, about 20%, about 30%, about 40%, about 50%, about 60%, or about 70% PVP).

In some embodiments, the dissolvable base comprises between about 1% and about 35% PVA (e.g., e.g., about 1%, about 2%, about 3%, about 4%, about 5%, about 6%, about 7%, about 8%, about 9%, about 10%, about 15%, about 20%, about 25%, about 30%, or about 35% PVA).

In some embodiments, the dissolvable base comprises between about 1% and about 75% hyaluronate (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% hyaluronate).

In some embodiments, the dissolvable base comprises between about 1% and about 75% maltose (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% maltose).

In some embodiments, the dissolvable base comprises between about 1% and about 75% methyl cellulose (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% methyl cellulose).

In some embodiments, the dissolvable base layer may comprise 40% hydrolyzed gelatin, 10% sucrose w/v in deionized (DI) water. Optionally, the base layer may include 1% low-viscosity carboxymethylcellulose (CMC), which may reduce brittleness. In some embodiments, the dissolvable base layer may comprise polyvinylpyrrolidone (PVP) of 10 kD MW at up to 50% w/v in DI water; polyvinyl alcohol (PVA) 87% hydrolyzed at 13 kD MW at up to 20% in DI water; or CMC at up to 10% in DI water. The following combinations may also be suitable for use in the fabrication of a dissolvable base layer: 30% PVP and 10% PVA; 37% PVP, 5% PVA, and 15% sucrose; or various other proportions of PVP, PVA, and sucrose.

The dissolvable base layer can be any suitable dimension, size, or shape. For example, the dissolvable base layer may have a dimension, size, or shape suitable for accommodating the microneedle array, and/or to better suit the intended site of application. In some embodiments, the shape of the dissolvable base layer comprises a circle. In some embodiments, the shape of the dissolvable base layer comprises a rectangle (e.g., a square, or rectangular strip). The dissolvable base layer may have rounded corners (e.g., a square or rectangle with rounded corners), or sharp corners (e.g., a square or rectangle with sharp corners).

In some embodiments, the dissolvable base layer has a diameter of between about 5 mm and about 50 mm, e.g., between about 6 mm and about 40 mm, between about 8 mm and about 35 mm, between about 10 mm and about 30 mm, between about 11 mm and about 25 mm, between about 12 mm and about 24 mm, between about 10 mm and about 20 mm, or between about 10 mm and about 15 mm. In some embodiments, the dissolvable base layer has a diameter of about 5 mm or more, e.g., about 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or more.

In some embodiments, the dissolvable base layer is a circle with a diameter of between about 8 mm and about 16 mm, e.g., about 8 mm, 9 mm, 10 mm, 11 mm, 12 mm, 13 mm, 14 mm, 15 mm, or 16 mm. In some embodiments, the dissolvable base layer is a square with dimensions of about 8 mm×8 mm, about 9 mm×9 mm, about 10 mm×10 mm, about 11 mm×11 mm, about 12 mm×12 mm, about 13 mm×13 mm, about 14 mm×14 mm, about 15 mm×15 mm, or about 16 mm×16 mm. In some embodiments, the dissolvable base layer is rectangular (e.g., a rectangular strip), with a width of about 8 mm or more (e.g., a width of about 9 mm, about 10 mm, about 11 mm, about 12 mm, about 13 mm, or more) and a length that is larger than the width, e.g., a length of about 10 mm or more (e.g., a length of about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, or more).

The thickness of the dissolvable base layer may be adjusted according to the particular microneedle device, and/or intended application of the microneedle device. In some embodiments, the thickness of the dissolvable base layer is between about 0.5 and 1 mm, e.g., between about 0.55 mm and 0.95 mm, between about 0.60 mm and 0.90 mm, between about 0.65 mm and 0.85 mm, or between about 0.70 mm and 0.80 mm. In some embodiments, the thickness of the dissolvable base layer is about 0.50 mm or more, e.g., about 0.55 mm, about 0.60 mm, about 0.65 mm, about 0.70 mm, about 0.75 mm, about 0.80 mm, about 0.85 mm, about 0.90 mm, about 0.95 mm, or more.

The dissolvable base layer may comprise one or more layers, e.g., by using more than one base layer solution in the preparation of the microneedle device, using methods described herein. In some embodiments, the dissolvable base layer comprises a single layer. In some embodiments, the dissolvable base layer comprises two layers. In some embodiments, the dissolvable base layer comprises three layers. In some embodiments, the dissolvable base layer comprises four or more layers. In some embodiments, the dissolvable base layer comprises more than one layer, wherein one or more of the layers is different, e.g., when each base layer was formed using one or more different compositions of base layer solutions. In some embodiments, the dissolvable base layer comprises more than one layer, wherein each of the layers is different.

In some embodiments, the dissolvable base layer is approximately 12 mm square and 0.75 mm thick. In some embodiments, the dissolvable base layer can cover the entire patch (e.g., microneedle patch). In some embodiments, the dimension of the base layer can be a 12 mm diameter circle, or a 12×12 mm square.

### Silk Fibroin Tip

The methods provided herein can be used to fabricate silk fibroin tips, e.g., silk fibroin-based implantable sustained-release and/or burst-release tips, of any dimensions. The silk fibroin tips can be configured to comprise, and release, an effective amount of a therapeutic agent (e.g., an anti-cancer agent, an immunomodulatory agent, or a combination thereof).

In some embodiments, the silk fibroin tips have dimensions ranging from about 75 μm to about 800 μm in height/length (e.g., about 75, about 100 μm, about 125 μm, about 150 μm, about 250 μm to about 300 μm, about 300 μm to about 350 μm, about 350 μm to about 400 μm, about 400 μm to about 450 μm, about 450 μm to about 500 μm, about 500 μm to about 550 μm, about 550 μm to about 600 μm, about 600 μm to about 650 μm, about 650 μm to about 700 μm, about 700 μm to about 750 μm, about 750 μm, to about 800 μm).

In some embodiments, the silk fibroin tip, e.g., implantable tip, can have a diameter of any size, e.g., based upon the type of biological barrier (e.g., skin layer, tumor, tissue, cell membrane, mucous surface, oral cavity, or buccal cavity) intended to be pierced by the tip. In some embodiments, the silk fibroin tips have a tip radius of about 10 μm or less (e.g., between about 1 μm and about 10 μm, e.g., about 1 μm or less, about 2 μm or less, about 3 μm or less, about 4 μm or less, about 5 μm or less, about 6 μm or less, about 7 μm or less, about 8 μm or less, about 9 μm or less, or about 10 μm or less). In embodiments, the tip can have a dimension (e.g., a diameter) ranging from about 50 nm to about 50 μm (e.g., about 50 nm to about 250 nm, about 250 nm to about 500 nm, about 500 to about 750 nm, about 750 nm to about 1 μm, about 1 μm to about 5 μm, about 5 μm to about 10 μm, about 10 μm to about 15 μm, about 15 μm to about 20 μm, about 20 μm to about 25 μm, about 25 μm to about 30 μm, about 30 μm to about 35 μm, about 35 μm to about 40 μm, about 40 μm to about 45 μm, or about 45 μm to about 50 μm). It can be understood that there is no fundamental limitation preventing the tips from having even smaller diameters (e.g., the limit of silk replica casting has been demonstrated with a resolution of tens of nm, see, e.g., Perry et al., 20 Adv. Mat. 3070 (2008)).

In some embodiments, the sharpness of the silk fibroin tip point, e.g., implantable sustained-release tip point, is described herein in terms of tip radius. The molds used in the fabrication of the microneedles described herein are designed to have a tip radius between about 0.5 μm to about 10 μm (e.g., about 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, or 10 μm). In some embodiments, the tip radius is between about 20 μm to about 25 μm (e.g., about 20 μm, 21 μm, 22 μm, 23 μm, 24 μm, or 25 μm). Without being bound by theory, it can be understood that blunter needles may require more force to penetrate the epidermis. In embodiments, other dimensions of a silk fibroin tip, e.g., an implantable sustained-release tip, may be controlled by the shape of the mold and fill volume. In some embodiments, a silk fibroin tip, e.g., an implantable sustained-release tip, can have an included angle between about 5 degrees and about 45 degrees (e.g., about 5, 6, 7, 8, 9, 10, 15, 20, 25, 30, 35, 40, or 45 degrees). In some embodiments, a silk fibroin tip, e.g., an implantable sustained-release tip, can have an included angle between about 15 degrees and 45 degrees (e.g., about 15 degrees, about 16 degrees, about 17 degrees, about 18 degrees, about 19 degrees, about 20 degrees, about 21 degrees, about 22 degrees, about 23 degrees, about 24 degrees, about 25 degrees, about 26 degrees, about 27 degrees, about 28 degrees, about 29 degrees, about 30 degrees, about 31 degrees, about 32 degrees, about 33 degrees, about 34 degrees, about 35 degrees, about 36 degrees, about 37 degrees, about 38 degrees, about 39 degrees, about 40 degrees, about 41 degrees, about 42 degrees, about 43 degrees, about 44 degrees, or about 45 degrees.

In embodiments, the height of a silk fibroin tip, e.g., an implantable sustained-release tip, may depend on the formulation and fill volume (e.g., fill volume or droplet dispensing volume), which can influence the surface tension and drying kinetics. In some embodiments, the height of the tip may extend to half of the full height of the microneedle. In some embodiments, the height of a silk fibroin tip, e.g., an implantable sustained-release tip, is between about 75 μm to about 475 μm (e.g., about 75, about 100 μm, about 125 μm, about 150 μm, about 175 μm, about 200 μm, about 225 μm, about 250 μm, about 275 μm, about 300 μm, about 325 μm, about 375 μm, about 400 μm, about 425 μm, or about 475 μm). In some embodiments, a portion of the tip comprises a thin “shell”-like layer roughly between about 5-10 μm thick (e.g., about 5, 6, 7, 8, 9, or 10 μm thick). In some embodiments, a silk fibroin tip, e.g., an implantable sustained-release tip, may dry to a more solid construct with a minimal “shell” wherein the height may be closer to 150 μm (e.g., between about 50 μm and about 200 μm) and the thickness >50 μm (e.g., between about 25 μm and about 75 μm).

Further, the microneedles of the present disclosure can take advantage of art known techniques developed, e.g., to functionalize silk fibroin (e.g., active agents such as dyes and sensors). See, e.g., U.S. Pat. No. 6,287,340, Bioengineered anterior cruciate ligament; WO 2004/000915, Silk Biomaterials & Methods of Use Thereof; WO 2004/001103, Silk Biomaterials & Methods of Use Thereof; WO 2004/062697, Silk Fibroin Materials & Use Thereof; WO 2005/000483, Method for Forming Inorganic Coatings; WO 2005/012606, Concentrated Aqueous Silk Fibroin Solution & Use Thereof; WO 2011/005381, Vortex-Induced Silk fibroin Gelation for Encapsulation & Delivery; WO 2005/123114, Silk-Based Drug Delivery System; WO 2006/076711, Fibrous Protein Fusions & Uses Thereof in the Formation of Advanced Organic/Inorganic Composite Materials; U.S. Application Pub. No. 2007/0212730, Covalently Immobilized Protein Gradients In Three-Dimensional Porous Scaffolds; WO 2006/042287, Method for Producing Biomaterial Scaffolds; WO 2007/016524, Method for Stepwise Deposition of Silk Fibroin Coatings; WO 2008/085904, Biodegradable Electronic Devices; WO 2008/118133, Silk Microspheres for Encapsulation & Controlled Release; WO 2008/108838, Microfluidic Devices & Methods for Fabricating Same; WO 2008/127404, Nanopatterned Biopolymer Device & Method of Manufacturing Same; WO 2008/118211, Biopolymer Photonic Crystals & Method of Manufacturing Same; WO 2008/127402, Biopolymer Sensor & Method of Manufacturing Same; WO 2008/127403, Biopolymer Optofluidic Device & Method of Manufacturing the Same; WO 2008/127401, Biopolymer Optical Wave Guide & Method of Manufacturing Same; WO 2008/140562, Biopolymer Sensor & Method of Manufacturing Same; WO 2008/127405, Microfluidic Device with Cylindrical Microchannel & Method for Fabricating Same; WO 2008/106485, Tissue-Engineered Silk Organs; WO 2008/140562, Electroactive Biopolymer Optical & Electro-Optical Devices & Method of Manufacturing Same; WO 2008/150861, Method for Silk Fibroin Gelation Using Sonication; WO 2007/103442, Biocompatible Scaffolds & Adipose-Derived Stem Cells; WO 2009/155397, Edible Holographic Silk Products; WO 2009/100280, 3-Dimensional Silk Hydroxyapatite Compositions; WO 2009/061823, Fabrication of Silk Fibroin Photonic Structures by Nanocontact Imprinting; WO 2009/126689, System & Method for Making Biomaterial Structures.

In various embodiments, the silk fibroin-based microneedle tips can further comprise at least one additional therapeutic agent, wherein the additional therapeutic agent can be dispersed throughout the microneedle or form at least a portion of the microneedle tip. In some embodiments, the additional therapeutic agent is useful in the treatment of a disease and/or disorder described herein, such a cancer. Optionally the silk fibroin-based microneedle tips can further comprise an excipient and/or adjuvant, as described herein.

In embodiments, the microneedle tip can be fabricated from silk fibroin and may comprise a therapeutic agent described herein (e.g., an anti-cancer agent, an immunomodulatory agent, or a combination thereof). In some embodiments, the tip can be designed to be deployed into the dermis layer of the skin (e.g., not into the subcutaneous space), as the population of professional antigen presenting cells in the dermis is much higher than in the subcutaneous space. In humans, the dermis ranges from about 1000-2000 μm (e.g., about 1-2 mm) thick based on location and patient age and health. In rodents, the dermis is much thinner (e.g., mice ˜100-300 μm, and rats ˜800-1200 μm). Without wishing to be bound by theory, with a 650 μm tall microneedle a tip, e.g., an implantable sustained-release tip, may be deployed at a depth of between about 100 μm and about 600 μm to achieve the controlled- or sustained-release of therapeutic agent as described herein.

Without being bound by theory, the molecular weight of the silk fibroin solution used in the fabrication of a microneedle described herein can function as a control factor to modulate the release of a therapeutic agent (e.g., an anti-cancer agent, an immunomodulatory agent, or a combination thereof) from the tip. In some embodiments, a higher molecular weight silk fibroin solutions can favor a slower controlled- or sustained-release (e.g., reducing the amount of an initial burst (e.g., the amount released on Day 0) by at least about 10% and then releasing additional antigen over at least about the next 4 days.). In some embodiments, the controlled- or sustained-release of an anti-cancer agent, an immunomodulatory agent, or a combination thereof, from the tip may be over at least about 4 days (e.g., about 4, 5, 6, 7, 8, 9, 10, 11, 12, 13, or 14 or more days, e.g., between about 4 days and about 15 days, e.g., between about 1-2 weeks, about 1-3 weeks, or about 1-4 weeks). In some embodiments, the release occurs over about 1 week to about 2 weeks.

In embodiments, the silk fibroin solution used in the fabrication of a microneedle described herein can be a low molecular weight silk fibroin composition comprising a population of silk fibroin fragments having a range of molecular weights, characterized in that: no more than 15% of the total number of silk fibroin fragments in the population has a molecular weight exceeding 200 kDa, and at least 50% of the total number of the silk fibroin fragments in the population has a molecular weight within a specified range, wherein the specified range is between about 3.5 kDa and about 120 kDa, or between about 5 kDa and about 125 kDa. Stated another way, the silk fibroin solution used in the fabrication of a microneedle described herein can comprise a population of silk fibroin fragments having a range of molecular weights, characterized in that: no more than 15% of the total moles of silk fibroin fragments in the population has a molecular weight exceeding 200 kDa, and at least 50% of the total moles of the silk fibroin fragments in the population has a molecular weight within a specified range, wherein the specified range is between about 3.5 kDa and about 120 kDa, or between about 5 kDa and about 125 kDa (see, e.g., WO2014/145002, incorporated herein by reference herein).

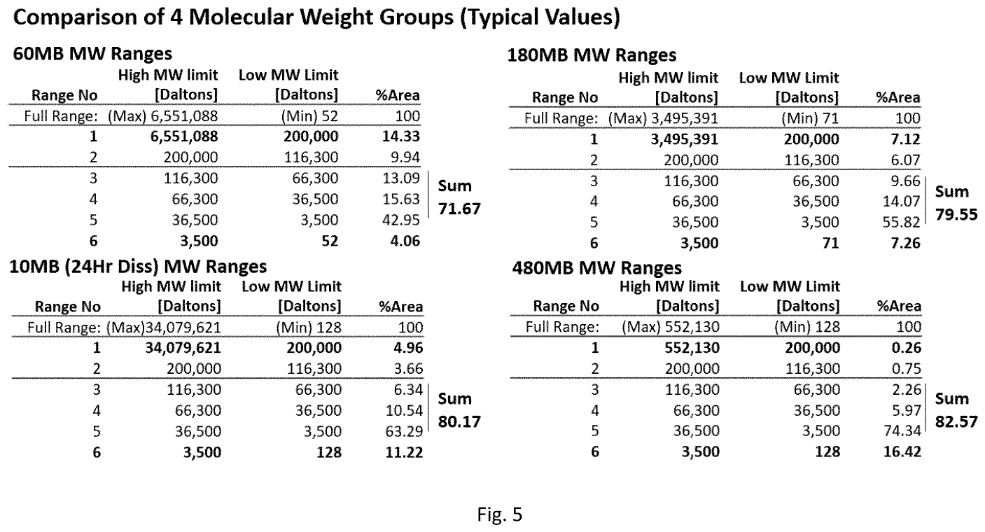

Exemplary silk fibroin (e.g., regenerated silk fibroin) solutions may have different molecular weight profiles, e.g., as determined by size exclusion chromatography (SEC) methods (see, e.g., FIG. 5). In some embodiments, the silk fibroin solutions can be prepared, e.g., according to established methods. In some embodiments, pieces of cocoons from the silkworm Bombyx mori were first boiled in 0.02 M Na₂CO₃ to remove sericin protein which is present in unprocessed, natural silk, prior to analysis by SEC. In some embodiments, silk fibroin composition can be a composition or mixture produced by degumming cocoons from the silkworm Bombyx mori at an atmospheric boiling temperature for about 480 minutes or less, e.g., less than 480 minutes, less than 400 minutes, less than 300 minutes, less than 200 minutes, less than 180 minutes, less than 120 minutes, less than 100 minutes, less than 60 minutes, less than 50 minutes, less than 40 minutes, less than 30 minutes, less than 20 minutes, less than 10 minutes or shorter. In one embodiment, the silk fibroin composition can be a composition or mixture produced by degumming silk cocoon at an atmospheric boiling temperature in an aqueous sodium carbonate solution for about 480 minutes or less, e.g., less than 480 minutes, less than 400 minutes, less than 300 minutes, less than 200 minutes, less than 180 minutes, less than 120 minutes, less than 100 minutes, less than 60 minutes, less than 50 minutes, less than 40 minutes, less than 30 minutes, less than 20 minutes, less than 10 minutes or shorter.

In some embodiments, the silk fibroin solution may be a 10-minute boil (10 MB), a 60-minute boil (60 MB), a 120-minute boil (120 MB), a 180-minute boil (180 MB), or a 480-minute boil (480 MB) silk fibroin solution (see, e.g., FIG. 5). In some embodiments, a therapeutic agent, such as an anti-cancer agent, an immunomodulatory agent, or a combination thereof, can be formulated in a 1% w/v to about 10% w/v (e.g., about 1, 2, 3, 4, 5, 6, 7, 8, 9, or 10% w/v) 10 MB silk fibroin solution. In some embodiments, an a therapeutic agent, such as an anti-cancer agent, an immunomodulatory agent, or a combination thereof, can be formulated in a 1% w/v to about 10% w/v (e.g., about 1, 2, 3, 4, 5, 6, 7, 8, 9, or 10% w/v) 60 MB silk fibroin solution. In some embodiments, a therapeutic agent, such as an anti-cancer agent, an immunomodulatory agent, or a combination thereof, can be formulated in a 1% w/v to about 10% w/v (e.g., about 1, 2, 3, 4, 5, 6, 7, 8, 9, or 10% w/v) 120 MB silk fibroin solution. In some embodiments, a therapeutic agent, such as an anti-cancer agent, an immunomodulatory agent, or a combination thereof, can be formulated in a 1% w/v to about 10% w/v (e.g., about 1, 2, 3, 4, 5, 6, 7, 8, 9, or 10% w/v) 180 MB silk fibroin solution. In some embodiments, a therapeutic agent, such as an anti-cancer agent, an immunomodulatory agent, or a combination thereof, can be formulated in a 1% w/v to about 10% w/v (e.g., about 1, 2, 3, 4, 5, 6, 7, 8, 9, or 10% w/v) 480 MB silk fibroin solution.

In some embodiments, a silk fibroin tip, e.g., an implantable sustained-release tip, may include silk fibroin in an amount of about 0.5 μg to about 500 μg silk fibroin. In some embodiments, a silk fibroin tip, e.g., an implantable sustained-release tip, may include silk fibroin in an amount of about 0.5 μg to about 5 μg, or about 1 μg to about 10 μg, or about 5 μg to about 15 μg, or about 10 μg to about 20 μg, or about 15 μg to about 25 μg, or about 20 μg to about 30 μg, or about 25 μg to about 35 μg, or about 30 μg to about 40 μg, or about 35 μg to about 45 μg, or about 40 μg to about 50 μg, or about 45 μg to about 55 μg, or about 50 μg to about 60 μg, or about 55 μg to about 65 μg, or about 60 μg to about 70 μg, or about 65 μg to about 75 μg, or about 70 μg to about 80 μg, or about 75 μg to about 85 μg, or about 80 μg to about 90 μg, or about 85 μg to about 95 μg, or about 90 μg to about 100 μg, or about 95 μg to about 150 μg, or about 125 μg to about 175 μg, or about 150 μg to about 200 μg, or about 225 μg to about 275 μg, or about 250 μg to about 300 μg, or about 325 μg to about 375 μg, or about 350 μg to about 400 μg, or about 425 μg to about 475 μg, or about 450 μg to about 500 μg. In some embodiments, a silk fibroin tip, e.g., an implantable sustained-release tip, may include silk fibroin in an amount of at least about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin. In some embodiments, a silk fibroin tip, e.g., an implantable sustained-release tip, may include silk fibroin in an amount of at most about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin. In some embodiments, a silk fibroin tip, e.g., an implantable sustained-release tip, may include silk fibroin in an amount of about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin.

In some embodiments, a silk fibroin tip, e.g., an implantable sustained-release tip, includes about 1% to about 75%, about 1% to about 5%, about 10% to about 60%, about 15% to about 50%, or about 20% to about 40% by weight, of silk fibroin. In some embodiments, a silk fibroin tip, e.g., an implantable sustained-release tip, includes at least about 1, 2, 3, 4, 5, 6, 7, 8, 9, 10, 15, 20, 25, 30, 35, 40, 45, 50, 55, 60, 65, 70, or 75% by weight, of silk fibroin. In some embodiments, a silk fibroin tip, e.g., an implantable sustained-release tip, includes at most about 1, 2, 3, 4, 5, 6, 7, 8, 9, 10, 15, 20, 25, 30, 35, 40, 45, 50, 55, 60, 65, 70, or 75% by weight, of silk fibroin. In some embodiments, a silk fibroin tip, e.g., an implantable sustained-release tip, includes about 1, 2, 3, 4, 5, 6, 7, 8, 9, 10, 15, 20, 25, 30, 35, 40, 45, 50, 55, 60, 65, 70, or 75% by weight, of silk fibroin.

Without being bound by theory, the primary tunability of a silk fibroin tip, e.g., an implantable sustained-release tip, is its crystallinity, measured via beta-sheet content (intermolecular and intramolecular P-sheet). This impacts the solubility of the silk tip matrix and the ability of antigen to be retained. With the increased R-sheet content, the tip also becomes more mechanically strong. Specific therapeutic agent (e.g., anti-cancer agent and/or immunomodulatory agent) release profiles are achieved through modulation of the crystallinity and the diffusivity of the silk matrix. This is accomplished through both silk input material and formulation as well as post-treatment to increase crystallinity (e.g. water annealing, methanol/solvent annealing). In some embodiments, the silk fibroin tip, e.g., implantable controlled- or sustained-release microneedle tip, comprises a beta-sheet content of between about 10% and about 60% (e.g., about 10%, about 20%, about 30%, about 40%, about 50%, about 60%), e.g., as based on a “crystallinity index,” e.g., a “crystallinity index” known in the art. In some embodiments, the silk fibroin tip, e.g., implantable controlled- or sustained-release microneedle tip, can be formulated as a particle (e.g., a microparticle and/or a nanoparticle).

In another aspect, the present disclosure features microneedles (e.g., silk-fibroin based microneedles) that can stabilize a therapeutic agent (e.g., an anti-cancer agent, an immunomodulatory agent, or both) disposed in the microneedle, e.g., due in part to the thermostabilization properties of the microneedle composition (e.g., the thermostabilization properties of the silk fibroin composition). In some embodiments, an anticancer-agent, an immunomodulatory agent, or both (e.g., an anticancer agent or immunomodulatory agent described herein) is stabilized by a microneedle or microneedle device described herein. Without wishing to be bound by theory, the ability of the microneedles or microneedle devices of the present disclosure to stabilize a therapeutic agent can facilitate storage of the microneedle devices, e.g., to prevent loss of bioactivity of the therapeutic agent during storage. Further, the bioactivity of the therapeutic agent can be maintained after administration of the microneedle or microneedle device. For example, the therapeutic agent can be stabilized within the silk fibroin tip, e.g., to prevent loss of bioactivity during the period of release (e.g., controlled release) at body temperature, following administration of the microneedle or microneedle device.

In some embodiments, the anti-cancer agent and/or immunomodulatory agent retains at least 50% (e.g., about 50%, 60%, 70%, 80%, 90%, 92%, 94%, 96%, 98%, 99%, 99.5%, or more) of its original bioactivity after storage for a period of 2 or more weeks, e.g., at room temperature (e.g., about 25° C.). In some embodiments, the anti-cancer agent and/or the immunomodulatory agent retains at least 70%, 80%, or 90% of its original bioactivity after storage at about 25° C., for at least about 2 weeks (e.g., for about 2 weeks, about 3 weeks, about 4 weeks, about 5 weeks, about 6 weeks, about 7 weeks, about 8 weeks, about 9 weeks, about 10 weeks, about 11 weeks, or about 12 weeks). In some embodiments, the anti-cancer agent and/or the immunomodulatory agent retains at least 60%, 70%, or 80% of its original bioactivity after storage at about 37° C., for at least about 2 weeks (for about 2 weeks, about 3 weeks, about 4 weeks, about 5 weeks, about 6 weeks, about 7 weeks, about 8 weeks, about 9 weeks, about 10 weeks, about 11 weeks, or about 12 weeks). In some embodiments, the anti-cancer agent and/or the immunomodulatory agent retains at least 50%, 60%, or 70% of its original bioactivity after storage at about 45° C., for at least about 2 weeks (for about 2 weeks, about 3 weeks, about 4 weeks, about 5 weeks, about 6 weeks, about 7 weeks, about 8 weeks, about 9 weeks, about 10 weeks, about 11 weeks, or about 12 weeks).

In some embodiments, the silk-fibroin based microneedles of the present disclosure stabilize a cytokine. For example, silk-fibroin can stabilize cytokines (e.g., interleukins, such as IL-2) during storage at a range of temperatures (e.g., storage at 4° C., room temperature (e.g., 25° C.), or 37° C.), including for extended periods of time (e.g., 1, 2, 3, 4, 5, 6, 7, 8, 9, 10, 11, 12, 13, or 14 or more days, 1, 2, 3, or 4 or more weeks, 1, 2, 3, 4, 5, 6, 7, 8, 9, 10, 11, or 12 or more months, or 1, 2, 3, or 4 or more years). In some embodiments, the silk-fibroin based microneedles stabilize an interleukin. In some embodiments, the silk-fibroin based microneedles stabilize interleukin-2 (IL-2). In some embodiments, the silk-fibroin based microneedles stabilize IL-2 at 4° C. over a period of at least 14 days. In some embodiments, the silk-fibroin based microneedles stabilize IL-2 at room temperature (about 25° C.) over a period of at least 14 days. In some embodiments, the silk-fibroin based microneedles stabilize IL-2 at body temperature (about 37° C.) over a period of at least 14 days.

### Microneedle Dimensions

In some embodiments, the present disclosure provides silk fibroin-based microneedles, and devices comprising the same, that have various dimensions and geometries.

In embodiments, the length of the silk fibroin-based microneedles can be fabricated sufficiently long enough to enable delivery (e.g., implantation) of a silk fibroin tip comprising a therapeutic agent (e.g., an anti-cancer agent, an immunomodulatory agent, or a combination thereof), to a desired depth within a biological barrier (e.g., skin), e.g., to induce an anti-cancer response, e.g., to induce an immune response.

In some embodiments, the biological barrier is a tumor and/or a skin lesion.

In some embodiments, the length of the silk fibroin-based microneedle can be between about 350 μm to about 1500 μm (e.g., about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm).

In some embodiments, the silk fibroin-based microneedle length is sufficient to enable delivery to the epidermis (e.g., about 10 μm to 120 μm below the skin surface).

In some embodiments, the silk fibroin-based microneedle length is sufficient to enable delivery to the dermis (e.g., about 60 μm to about 2.1 mm below the skin surface).

In some embodiments, the silk fibroin-based microneedle length is sufficient to enable delivery to the eye (e.g., about 10 μm to 120 μm below the eye surface).

In some embodiments, the silk fibroin-based microneedle length is sufficient to enable delivery to a tumor (e.g., about 10 μm to 120 μm below the tumor surface).

In some embodiments, the microneedle is configured to implant the silk fibroin tip into a biological barrier of a subject at a depth (e.g., a max penetration depth of the distal part of tip) of between about 100 μm and about 600 μm. In some embodiments, the length of the microneedle is between about 350 μm to about 1500 μm. In some embodiments, the height of the silk fibroin tip may extend to approximately half of the full height of the microneedle. In some embodiments, the height of the silk fibroin tip is between about 75 μm to about 475 μm. In some embodiments, the silk fibroin tip comprises a tip radius between about 0.5 μm to about 25 μm. In some embodiments, the silk fibroin tip comprises a tip radius between about 5 μm to about 10 μm. In some embodiments, the silk fibroin tip comprises an angle between about 5 degrees and about 45 degrees.

In some embodiments, the silk fibroin tip, e.g., implantable tip, can have a diameter of any size, e.g., based upon the type of biological barrier (e.g., skin layer) intended to be pierced by the tip. In some embodiments, the silk fibroin tips have a tip radius of about 10 μm or less (e.g., between about 1 μm and about 10 μm, e.g., about 1 μm or less, about 2 μm or less, about 3 μm or less, about 4 μm or less, about 5 μm or less, about 6 μm or less, about 7 μm or less, about 8 μm or less, about 9 μm or less, or about 10 μm or less). In embodiments, the tip can have a dimension (e.g., a diameter) ranging from about 50 nm to about 50 μm (e.g., about 50 nm to about 250 nm, about 250 nm to about 500 nm, about 500 to about 750 nm, about 750 nm to about 1 μm, about 1 μm to about 5 μm, about 5 μm to about 10 μm, about 10 μm to about 15 μm, about 15 μm to about 20 μm, about 20 μm to about 25 μm, about 25 μm to about 30 μm, about 30 μm to about 35 μm, about 35 μm to about 40 μm, about 40 μm to about 45 μm, or about 45 μm to about 50 μm). It can be understood that there is no fundamental limitation preventing the tips from having even smaller diameters (e.g., the limit of silk replica casting has been demonstrated with a resolution of tens of nm, see, e.g., Perry et al., 20 Adv. Mat. 3070 (2008)).

In some embodiments, the sharpness of the silk fibroin tip point, e.g., implantable sustained-release tip point, is described herein in terms of tip radius. The molds used in the fabrication of the microneedles described herein are designed to have a tip radius between about 0.5 μm to about 10 μm (e.g., about 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, or 10 μm). In some embodiments, the tip radius is between about 20 μm to about 25 μm (e.g., about 20 μm, 21 μm, 22 μm, 23 μm, 24 μm, or 25 μm). Without being bound by theory, it can be understood that blunter needles may require more force to penetrate the epidermis. In embodiments, other dimensions of a silk fibroin tip, e.g., an implantable sustained-release tip, may be controlled by the shape of the mold and fill volume. In some embodiments, a silk fibroin tip, e.g., an implantable sustained-release tip, can have an included angle between about 5 degrees and about 45 degrees (e.g., about 5, 6, 7, 8, 9, 10, 15, 20, 25, 30, 35, 40, or 45 degrees). In some embodiments, the tip can have an included angle between about 15 degrees and 45 degrees (e.g., about 15 degrees, about 16 degrees, about 17 degrees, about 18 degrees, about 19 degrees, about 20 degrees, about 21 degrees, about 22 degrees, about 23 degrees, about 24 degrees, about 25 degrees, about 26 degrees, about 27 degrees, about 28 degrees, about 29 degrees, about 30 degrees, about 31 degrees, about 32 degrees, about 33 degrees, about 34 degrees, about 35 degrees, about 36 degrees, about 37 degrees, about 38 degrees, about 39 degrees, about 40 degrees, about 41 degrees, about 42 degrees, about 43 degrees, about 44 degrees, or about 45 degrees.

Without wishing to be bound by theory, a skilled artisan can adjust the silk fibroin-based microneedle length for a number of factors, including, without limitations, tissue thickness, e.g., skin thickness, (e.g., as a function of age, gender, location on body, subject species (e.g., human), drug delivery profile, diffusion properties of the therapeutic agent (e.g., the ionic charge and/or molecule weight, and/or shape of the therapeutic agent), or any combinations thereof.

However, without wishing to be bound by theory, with an approximately 650 μm tall microneedle a silk fibroin tip may be deployed (e.g., implanted) at a depth of between about 100 μm and about 600 μm within the dermis layer of the skin to a subject to achieve release (e.g., burst release or sustained release) of the therapeutic agent (e.g., an anti-cancer agent, an immunomodulatory agent, or a combination thereof) from the silk fibroin tip. In some embodiments, the microneedle may be about 800 μm tall (e.g., between about 500 μm and 1200 μm tall).

### Silk Fibroin-Based Microneedle Devices for Combination Therapy

In some embodiments, a silk fibroin-based microneedle device (e.g., microneedle patch) of the present disclosure can comprises a plurality of microneedles disclosed herein. The silk fibroin-based microneedle device (e.g., microneedle patch) can comprise a plurality of microneedles of the same type. Alternatively, the microneedle device can comprise a plurality of microneedles of different types.

For example, the silk fibroin-based microneedle device can comprise a plurality of microneedles comprising an anti-cancer agent. In some embodiments, the silk fibroin-based microneedle device can comprise a plurality of microneedles comprising a combination of anti-cancer agents (e.g., two, three, four, five, six, or more anti-cancer agents). For microneedles configured to deliver a combination of anti-cancer agents (e.g., two, three, four, five, six, or more anti-cancer agents), the microneedle device can comprise, e.g., a plurality of the same microneedle comprising the same combination (e.g., all) of the anti-cancer agents; or a plurality of different microneedles comprising different anti-cancer agents or different combinations of anti-cancer agents. In some embodiments, a microneedle in a plurality comprises one anti-cancer agent. In some embodiments, a microneedle in a plurality comprises two or more anti-cancer agents (e.g., two, three, four, five, six, or more anti-cancer agents).

In some embodiments, the silk fibroin-based microneedle device can comprise a plurality of microneedles comprising an immunomodulatory agent. In some embodiments, the silk fibroin-based microneedle device can comprise a plurality of microneedles comprising a combination of immunomodulatory agents (e.g., two, three, four, five, six, or more anti-cancer agents). For microneedles configured to deliver a combination of immunomodulatory agents (e.g., two, three, four, five, six, or more immunomodulatory agents), the microneedle device can comprise, e.g., a plurality of the same microneedle comprising the same combination (e.g., all) of the immunomodulatory agents; or a plurality of different microneedles comprising different immunomodulatory agents or different combinations of immunomodulatory agents. In some embodiments, a microneedle in a plurality comprises one immunomodulatory agent. In some embodiments, a microneedle in a plurality comprises two or more immunomodulatory agents (e.g., two, three, four, five, six, or more immunomodulatory agents).

In some embodiments, the silk fibroin-based microneedle device can comprise a plurality of microneedles comprising a combination of an anti-cancer agent and an immunomodulatory agent. For microneedles configured to deliver a combination of an anti-cancer agent and an immunomodulatory agent, the microneedle device can comprise, e.g., a plurality of the same microneedle comprising the same combination of an anti-cancer agent and an immunomodulatory agent; or a plurality of different microneedles comprising different combinations of an anti-cancer agent and an immunomodulatory agent.

In some embodiments, for microneedles configured to deliver a combination of an anti-cancer agent and an immunomodulatory agent, the microneedle device can comprise: a plurality of microneedles comprising one anti-cancer agent; a plurality of microneedles comprising two or more anti-cancer agents (e.g., two, three, four, five, six, or more anti-cancer agents); a plurality of microneedles comprising one immunomodulatory agent; a plurality of microneedles comprising two or more immunomodulatory agents (e.g., two, three, four, five, six, or more immunomodulatory agents); and/or a plurality of microneedles comprising both an anti-cancer agent (e.g., one, two, three, four, five, six, or more anti-cancer agents) and an immunomodulatory agent (e.g., one, two, three, four, five, six, or more immunomodulatory agents).

In some embodiments, a plurality of microneedles can be arranged in a random or predefined pattern to form a microneedle or patch, as described herein. The patch may comprise a carrier, backing, or “handle” layer adhered to the back of the base (see, e.g., FIG. 3). This layer can provide structural support and an area by which the patch can be handled and manipulated without disturbing the needle array.

Microneedle devices (e.g., microneedle patches) described herein may be designed to accommodate a varying number of microneedles. In some embodiments, the microneedle device comprises at least 50 microneedles, e.g., at least about 60, about 64, about 70, about 80, about 81, about 90, about 100, about 110, about 121, about 144, about 150, about 169, about 175, about 196, about 200, about 225, about 250, about 256, about 289, about 300, or more microneedles. In some embodiments, the microneedle device (e.g., microneedle patch) comprises between about 50 microneedles and 500 microneedles, e.g., between about 50 and 400 microneedles, between about 75 and about 300 microneedles, between about 100 and 200 microneedles, or between about 100 and 150 microneedles. The microneedles may be arranged in a grid, e.g., a square grid. In some embodiments, the microneedles are arranged in an 8×8 grid, a 9×9 grid, a 10×10 grid, an 11×11 grid, a 12×12 grid, a 13×13 grid, a 14×14 grid, a 15×15 grid, a 16×16 grid, or a 17×17 grid. In some embodiment the microneedles are arranged in an 11×11 grid. In some embodiments, the microneedle device has a pitch of between about 0.5 mm and 1.0 mm, e.g., about 0.55 mm, 0.60 mm, 0.65 mm, 0.70 mm, 0.75 mm, 0.80 mm, 0.85 mm, 0.95 mm, or 1 mm. In some embodiments, the microneedle device has a pitch of about 0.75 mm.

In some embodiments, the microneedle device comprises about 121 needles in an 11×11 square grid with approximately 0.75 mm pitch. In some embodiments, the microneedle device (e.g., microneedle patch) may comprise about 121 needles in an 11×11 square grid with approximately 0.75 mm pitch. In some embodiments, the microneedle device comprises individual needles that are cones approximately 0.65 mm long with a base diameter approximately 0.35 mm and an included angle of approximately 30°. In some embodiments, the microneedle device comprises individual needles having a silk fibroin tip, wherein the silk fibroin tip of the needle is sufficiently sharp to penetrate a biological barrier (e.g., skin). In some embodiments, the microneedle device comprises individual needles having a silk fibroin tip, wherein the radius of curvature of the silk fibroin tip should ideally be no more than 0.01 mm.

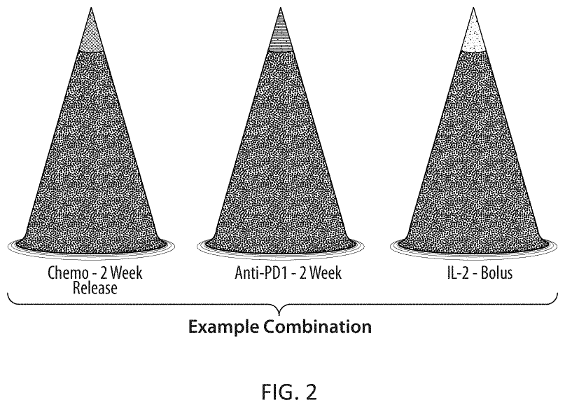

Exemplary microneedles and devices of the present disclosure are depicted in FIGS. 2-4.

### Release Kinetics

The disclosed silk fibroin-based microneedles can be configured to release a therapeutic agent or a combination of therapeutic agents (e.g., an anti-cancer agent, an immunomodulatory agent, or a combination thereof) according to various release kinetics.

In some embodiments, the silk fibroin-based microneedles can be configured to release an effective amount of the anti-cancer agent and/or the immunomodulatory agent to induce an anti-cancer response in a subject. In certain embodiments, release of the anti-cancer agent and/or the immunomodulatory agent enhances exposure of the subject's immune system to a neoantigen associated with a cancer, thereby inducing immune effector cells, e.g., T cells, that are specific for the neoantigen to be activated and/or expanded in the subject. Release of the anti-cancer agent and/or the immunomodulatory agent can facilitate the development of a prolonged immunity to a cancer in a subject.

In some embodiment, silk fibroin-based microneedles can be configured to release a therapeutic agent or a combination of therapeutic agents (e.g., an anti-cancer agent, an immunomodulatory agent, or a combination thereof) by burst release. In some embodiments, burst release comprises a rapid administration of the anti-cancer agent and/or the immunomodulatory agent to the subject. The burst release can comprise a rapid administration of a greater than 0% portion to about a 100% portion (e.g., about 1 to about 25%, about 25% to about 50%, about 50% to about 75%, about 75% to about 100%) of a total amount of anti-cancer agent and/or a total amount of immunomodulatory agent present in the silk fibroin tip. In some embodiments, the burst release is over a period of time comprising at least about 1 hour (e.g., about 1 to about 30 minutes, e.g., about 1, 2, 3, 4, 5, 6, 7, 8, 9, 10, 15, 20, or 24 hours).

In some embodiments, silk fibroin-based microneedles can be configured to release a therapeutic agent or a combination of therapeutic agents (e.g., an anti-cancer agent, an immunomodulatory agent, or a combination thereof) by sustained release. Examples of sustained release include, but are not limited to, zero order release, first order release, and second order release. In some embodiments, zero order release is a rate of release that is independent of the therapeutic agent concentration in the dosage form (e.g., microneedle). In some embodiments, zero order release is a release of therapeutic agent that is approximately constant over a period of time (e.g., a constant amount of therapeutic agent is released per unit time). In some embodiments, first order release is a rate of release that is a function of the amount of the therapeutic agent remaining in the dosage form (e.g., microneedle). In some embodiments, first order release is a release of a constant proportion, such as a percentage, of therapeutic agent from the dosage form (e.g., microneedle) per unit time. In some embodiments, second order release is where doubling the concentration of therapeutic agent in the dosage form quadruples the release rate. In some embodiments, sustained-released comprises a substantially continuous low dose administration of the anti-cancer agent and/or the immunomodulatory agent. The sustained release can comprise a continuous administration of about a greater than 0% portion to about a 100% portion (e.g., about 1 to about 25%, about 25% to about 50%, about 50% to about 75%, about 75% to about 100%) of a total amount of anti-cancer agent and/or a total amount of immunomodulatory agent present in the silk fibroin tip. In some embodiments, the sustained release is over a period of time comprising at least about 3 days (e.g., about 3, 4, 5, 6, 7, or more days, e.g., between about 5 days and about 10 days, e.g., between about 7 days and about 15 days, e.g., between about 1 to about 2 weeks, between about 1 to about 3 weeks, or between about 2 to about 4 weeks, e.g., between about 1 to about 3 months, e.g., between about 2 to about 4 months, e.g., between about 3 to about 6 months). In certain embodiments, the sustained release is over a period of time between about 7 days and about 15 days.

In some embodiments, the release (e.g., administration) of a therapeutic agent (e.g., an anti-cancer agent, an immunomodulatory agent, or a combination thereof) from a silk fibroin-based microneedle described herein can be facilitated by the diffusion of the therapeutic agent from the microneedle or a portion thereof.

In some embodiments, the release (e.g., administration) of a therapeutic agent (e.g., an anti-cancer agent, an immunomodulatory agent, or a combination thereof) from a silk fibroin-based microneedle described herein can be facilitated by the degradation (e.g., protease mediated degradation) of the microneedle or a portion thereof.

In some embodiments, the release (e.g., administration) of a therapeutic agent (e.g., an anti-cancer agent, an immunomodulatory agent, or a combination thereof) from a silk fibroin-based microneedle described herein can be facilitated by the dissolution of the microneedle or a portion thereof.

Without wishing to be bound by theory, the release of the anti-cancer agent can occur at substantially the same rate (e.g., concurrently) with the release of the immunomodulatory agent. In other embodiments, the release of the anti-cancer agent can occur at a different rate than the release rate of the immunomodulatory agent, such that anti-cancer agent is released substantially before or substantially after the release of the immunomodulatory agent.

### Therapeutic Agents

The present disclosure provides, in some embodiments, a silk fibroin-based microneedle comprising a therapeutic agent (e.g., an anti-cancer agent, an immunomodulatory agent, or a combination thereof). The present disclosure also provides combination treatment methods for administering a microneedle disclosed herein with an additional therapy. Non-limiting examples of therapeutic agents which can be incorporated into a silk fibroin-based microneedle of the present disclosure, and/or can be administered as a combination therapy with a fibroin-based microneedle of the present disclosure are disclosed below.

### Anti-Cancer Agents

In some embodiments, silk fibroin-based microneedle of the present disclosure can comprise and/or can be administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), eribulin (HALAVEN®), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™) paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In some embodiments, a silk fibroin-based microneedle of the present disclosure can comprise and/or can be administered in combination with an FDA approved targeted therapy. For example, for the treatment of a melanoma a silk fibroin-based microneedle of the present disclosure can comprise and/or can be administered in combination with Binimetinib (MEKTOVI®), Cobimetinib (COTELLIC®), Dabrafenib (TAFINLAR®), Encorafenib (BRAFTOVI®), Trametinib (MEKINIST®), and/or Vemurafenib (ZELBORAF®).

In some embodiments, for the treatment of a basal cell carcinoma a silk fibroin-based microneedle of the present disclosure can comprise and/or can be administered in combination with Sonidegib (ODOMZO®) and/or Vismodegib (Erivedge®).

In some embodiments, for the treatment of a breast cancer a silk fibroin-based microneedle of the present disclosure can comprise and/or can be administered in combination with Abemaciclib (VERZENIO®), Alpelsib (PIQRAY®), Olaparib (LYNPARZA®), Palbociclib (IBRANCE®), Ribociclib (KISQALI®), and/or Talazoparib (TALZENNA®).

In some embodiments, silk fibroin-based microneedle of the present disclosure can comprise and/or can be administered in combination with a biologic. Biologics useful in the treatment of cancers are known in the art and a silk fibroin-based microneedle, as described herein, may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); AROMASIN® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the silk fibroin-based microneedles of the present disclosure may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® Velcade (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, ado-trastuzumab emtansine (KADCYLA®), afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), atezolizumab (TECENTRIQ®), avelumab (BAVENCIO®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cemiplimab-rwlc (LIBTAYO®) cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), trastuzumab and hyaluronidase-oysk (HERCEPTIN HYLECTA®), tremelimumab, tucotuzumab celmoleukin, veltuzumab, volociximab, votumumab (HUMASPECT®), zalutumumab (HUMAX-EGFR®), and zanolimumab (HUMAX-CD4®).

In other embodiments, silk fibroin-based microneedle of the present disclosure can comprise and/or can be administered in combination with a viral cancer therapeutic agent. Exemplary viral cancer therapeutic agents include, but are not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/ASO4 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®). In other embodiments, silk fibroin-based microneedle of the present disclosure can comprise and/or can be administered in combination with Talimogene Laherparepvec (IMLYGIC®), an FDA approved melanoma treatment.

In other embodiments, silk fibroin-based microneedle of the present disclosure can comprise and/or can be administered in combination with a neoantigen vaccine. In certain embodiments, a neoantigen vaccine can be prepared as described, for example, in Schumacher et al. Science. 348(6230): 69-74, 2015, incorporated herein by reference in its entirety. In some embodiments, a microneedle disclosed herein can be used in a method for identifying a neoantigen, and/or in a method of preparing a neoantigen vaccine. Without wishing to be bound by theory, cancer neoantigens derived from random somatic mutations in tumor tissue represent an attractive type of target for cancer immunotherapies including cancer vaccines. Vaccination against the tumor-specific neoantigens minimizes the potential induction of central and peripheral tolerance as well as the risk of autoimmunity. (See e.g., Guo et al. Frontiers in Immunology. Vol. 9 Article 1499, 2018, which is incorporated herein by reference in its entirety). In certain embodiments, the application of a microneedle of the present disclosure to a tumor can result in the subject's immune system being exposed to tumor-specific neoantigens, and result in the development of a vaccination and immunity to cancers having the same, or similar, tumor-specific neoantigens.

In other embodiments, silk fibroin-based microneedle of the present disclosure can comprise and/or can be administered in combination with a nanopharmaceutical. Exemplary cancer nanopharmaceuticals include, but are not limited to, ABRAXANE® (paclitaxel bound albumin nanoparticles), CRLX101 (camptothecin (CPT) conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, silk fibroin-based microneedle of the present disclosure can comprise and/or can be administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate (see, e.g., Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte-Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®))

In some embodiments, silk fibroin-based microneedle of the present disclosure can comprise and/or can be administered in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-B inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074,nSorafenib Tosylate(Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In some embodiments, silk fibroin-based microneedle of the present disclosure can comprise and/or can be administered in combination with one of more of: an anti-angiogenic agent, or a vascular targeting agent or a vascular disrupting agent. Exemplary anti-angiogenic agents include, but are not limited to, vascular endothelial growth factor (VEGF) inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecules. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

In certain embodiments, the anti-cancer agent described herein can be formulated in a sustained release particle.

### Immunomodulatory Agents

### Checkpoint Inhibitors

In some embodiments, silk fibroin-based microneedle of the present disclosure can comprise and/or can be administered in combination with an immune checkpoint inhibitor. For example, in certain embodiments, a silk fibroin-based microneedle can be used to locally administer a therapeutic agent (e.g., ant anti-cancer agent and/or an immunomodulatory agent) to a tumor in a subject in combination with systemic administration (e.g., by injection) of a checkpoint inhibitor (e.g., an anti-PD1 antibody).

In embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR. See, e.g., Pardoll. Nat. Rev. Cancer 12.4(2012):252-64, incorporated herein by reference.

In some embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. (See, e.g., Hamid, O. et al. (2013) New England Journal of Medicine 369 (2): 134-44, U.S. Pat. No. 8,354,509 and WO2009/114335). Pidilizumab (also called CT-011 or Cure Tech) is a humanized IgG1k monoclonal antibody that binds to PD1. (See, e.g., WO2009/101611). In one embodiment, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In some embodiments, the immune checkpoint inhibitor is a PD-L1 inhibitor, e.g., an antibody molecule. In some embodiments, the PD-L1 inhibitor is YW243.55.570, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In one embodiment, the PD-LI inhibitor is an anti-PD-L1 antibody, e.g., YW243.55.570. The YW243.55.570 antibody is described, e.g., in WO 2010/077634. In one embodiment, the PD-L1 inhibitor is MDX-1105 (also called BMS-936559), which is described, e.g., in WO2007/005874. In one embodiment, the PD-LI inhibitor is MDPL3280A (Genentech/Roche), which is a human Fc-optimized IgG1 monoclonal antibody against PD-L1. See, e.g., U.S. Pat. No. 7,943,743 and U.S. Publication No.: 20120039906. In one embodiment, the inhibitor of PD-L1 is an antibody molecule having a sequence substantially identical or similar thereto, e.g., a sequence at least 85%, 90%, 95% identical or higher to the sequence of YW243.55.570, MPDL3280A, MEDI-4736, MSB-0010718C, or MDX-1105.

In embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In one embodiment, the immune checkpoint inhibitor is a LAG-3 inhibitor, e.g., an anti LAG-3 antibody molecule. In embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S. Publication No.: 2014/044728.

In embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### TLR Agonists

In some embodiments, silk fibroin-based microneedle of the present disclosure can comprise and/or can be administered in combination with a Toll-like receptor (TLR) agonist.

TLRs are a family of pattern recognition receptors that were initially identified as sensors of the innate immune system that recognize microbial pathogens. In humans, the TLRs include TLR-1, TLR-2, TLR-3, TLR-4, TLR-5, TLR-6, TLR-7, TLR-8, TLR-9, and TLR-10. TLR-1, -2, -4, -5, and -6, are expressed on the surface of cells and TLR-3, -7/8, and -9 are expressed with the ER compartment. Human dendritic cell subsets can be identified on the basis of distinct TLR expression patterns. The myeloid or “conventional” subset of human dendritic cells express TLRs 1-8 and the plasmacytoid subset of dendritic cells express only TLR-7 and TLR-9. Ligand binding to TLRs invokes a cascade of intra-cellular signaling pathways that induce the production of factors involved in inflammation and immunity. Upon stimulation, the myeloid subset and the plasmacytoid subset of human dendritic cells result in antigen-specific CD4+ and CD8+ T cell priming and activation of NK cells and T-cells, respectively.

In some embodiments, the TLR agonist is chosen from one or more of a TLR-1 agonist, a TLR-2 agonist, a TLR-3 agonist, a TLR-4 agonist, a TLR-5 agonist, a TLR-6 agonist, a TLR-7 agonist, a TLR-8 agonist, a TLR-9 agonist, a TLR-10 agonist, a TLR-1/2 agonist, a TLR-2/6 agonist, or a TLR-7/8 agonist. In one embodiment, the TLR agonist is a TLR7 agonist.

In some embodiments, the TLR agonist is imiquimod or 3-(2-methylpropyl)-3,5,8-triazatricyclo[7.4.0.02,6]trideca-1(9),2(6),4,7,10,12-hexaen-7-amine. Imiquimod or 3-(2-methylpropyl)-3,5,8-triazatricyclo[7.4.0.02,6]trideca-1(9),2(6),4,7,10,12-hexaen-7-amine can bind to and activate TLR-7 and/or TLR-8.

In some embodiments, the TLR agonist is 852A. 852A is disclosed, e.g., in Inglefield et al. J Interferon Cytokine Res. 2008; 28(4):253-63. 852A can bind to and activate TLR-7 and/or TLR-8.

In some embodiments, the TLR agonist is Bacille Calmette-Guérin (BCG). BCG can bind to and activate TLR-9.

In some embodiments, the TLR agonist is EMD 120108. EMD 120108 is a synthetic oligonucleotide containing phosphorothioate oligodeoxynucleotide. EMD 1201081 can bind to and activate TLR-9, e.g., in monocytes/macrophages, plasmacytoid dendritic cells (DCs) and B cells, initiating immune signaling pathways, activating B cells and inducing T-helper cell cytokine production.

In some embodiments, the TLR agonist is IMO-2055. IMO-2055 is a synthetic oligonucleotide containing unmethylated CpG dinucleotides. Mimicking unmethylated CpG sequences in bacterial DNA, IMO-2055 can bind to and activate TLR-9, e.g., in monocytes/macrophages, plasmacytoid dendritic cells (DCs) and B cells, initiating immune signaling pathways and activating B cells and DCs and inducing T-helper cell cytokine production.

Other exemplary TLR agonists that can be used in the combination include, e.g., TLR-1/2 agonists (e.g., Pam3Cys), TLR-2 agonists (e.g., CFA, MALP2, Pam2Cys, FSL-1, or Hib-OMPC), TLR-3 agonists (e.g., polyribosinic:polyribocytidic acid (Poly I:C), polyadenosine-polyuridylic acid (poly AU), polyinosinic-polycytidylic acid stabilized with poly-L-lysine and carboxymethylcellulose (Hiltonol®)), TLR-4 agonists (e.g., monophosphoryl lipid A (MPL), LPS, sialyl-Tn (STn)), TLR-5 agonists (e.g., bacterial flagellin), TLR-7 agonists (e.g., imiquimod), TLR-7/8 agonists (e.g., resiquimod or loxoribine), and TLR-9 agonists (e.g., unmethylated CpG dinucleotide (CpG-ODN)).

In another embodiment, the TLR agonist is used in combination with a GITR agonist, e.g., as described in WO2004/060319, and International Publication No.: WO2014/012479.

### STING Agonists

In some embodiments, silk fibroin-based microneedle of the present disclosure can comprise and/or can be administered in combination with a STING agonist.

In some embodiments, the STING agonist is cyclic dinucleotide, e.g., a cyclic dinucleotide comprising purine or pyrimidine nucleobases (e.g., adenosine, guanine, uracil, thymine, or cytosine nucleobases). In some embodiments, the nucleobases of the cyclic dinucleotide comprise the same nucleobase or different nucleobases.

In some embodiments, the STING agonist comprises an adenosine or a guanosine nucleobase. In some embodiments, the STING agonist comprises one adenosine nucleobase and one guanosine nucleobase. In some embodiments, the STING agonist comprises two adenosine nucleobases or two guanosine nucleobases.

In some embodiments, the STING agonist comprises a modified cyclic dinucleotide, e.g., comprising a modified nucleobase, a modified ribose, or a modified phosphate linkage.

In some embodiments, the modified cyclic dinucleotide comprises a modified phosphate linkage, e.g., a thiophosphate.

In some embodiments, the STING agonist comprises a cyclic dinucleotide (e.g., a modified cyclic dinucleotide) with 2′,5′ or 3′,5′ phosphate linkages. In some embodiments, the STING agonist comprises a cyclic dinucleotide (e.g., a modified cyclic dinucleotide) with Rp or Sp stereochemistry around the phosphate linkages.

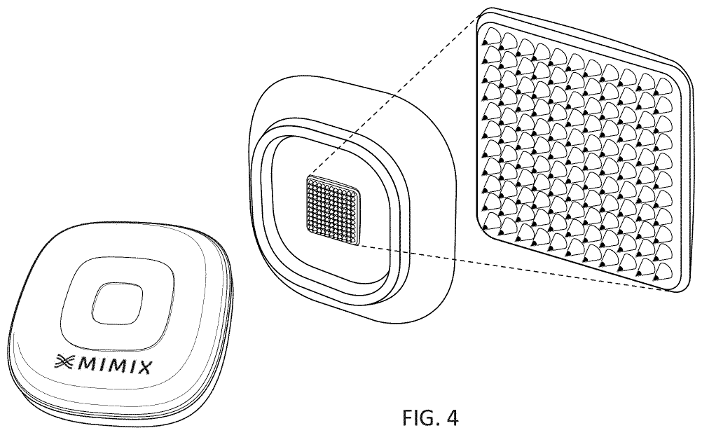

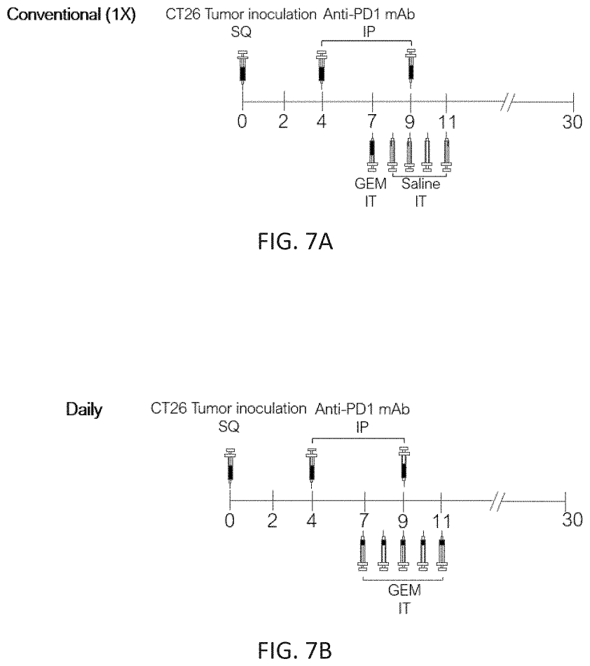

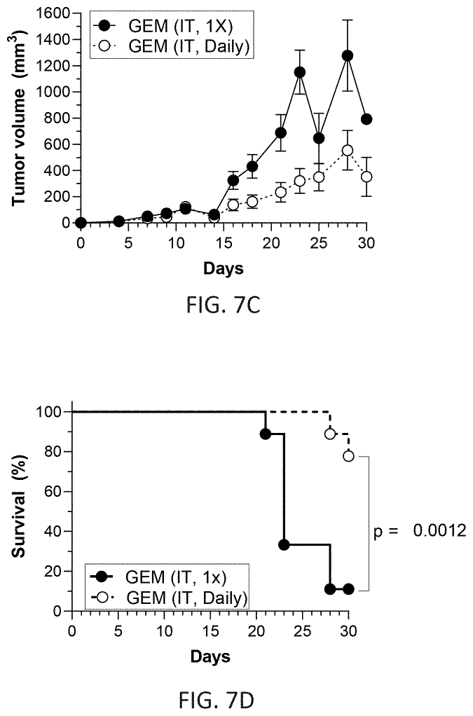

In some embodiments, the STING agonist is Rp,Rp dithio 2′,3′ c-di-AMP (e.g., Rp,Rp-dithio c-[A(2′,5′)pA(3′,5′)p]), or a cyclic dinucleotide analog thereof. In some embodiments, the STING agonist is a compound depicted in U.S. Patent Publication No. US2015/0056224 (e.g., a compound in FIG. 2c, e.g., compound 21 or compound 22). In some embodiments, the STING agonist is c-[G(2′,5′)pG(3′,5′)p], a dithio ribose O-substituted derivative thereof, or a compound depicted in FIG. 4 of PCT Publication Nos. WO 2014/189805 and WO 2014/189806. In some embodiments, the STING agonist is c-[A(2′,5′)pA(3′,5′)p] or a dithio ribose O-substituted derivative thereof, or is a compound depicted in FIG. 5 of PCT Publication Nos. WO 2014/189805 and WO 2014/189806. In some embodiments, the STING agonist is c-[G(2′,5′)pA(3′,5′)p], or a dithio ribose O-substituted derivative thereof, or is a compound depicted in FIG. 5 of PCT Publication Nos. WO 2014/189805 and WO 2014/189806. In some embodiments, the STING agonist is 2′-0-propargyl-cyclic-[A(2′,5′)pA(3′,5′)p] (2′-0-propargyl-ML-CDA) or a compound depicted in FIG. 7 of PCT Publication No. WO 2014/189806.

Other exemplary STING agonists are disclosed, e.g., in PCT Publication Nos. WO 2014/189805 and WO 2014/189806, and U.S. Publication No. 2015/0056225.

### RIG Agonists

In some embodiments, silk fibroin-based microneedle of the present disclosure can comprise and/or can be administered in combination with a RIG agonist (i.e., agonists of retinoic acid-inducible gene I (RIG-I), encoded by the gene DDX58). Exemplary RIG agonists are described in Elion et al. Oncotarget. 9(48): 29007-29017, 2018, which is incorporated herein by reference in its entirety.

### Cytokines

In some embodiments, silk fibroin-based microneedle of the present disclosure can comprise and/or can be administered in combination with a cytokine.

The cytokines are generally polypeptides that influence cellular activity, for example, through signal transduction pathways. Accordingly, a cytokine is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

Accordingly, in some embodiments, the cytokine molecule is an interleukin or a variant thereof, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine. In an embodiment, the cytokine molecule is IL-18.

The cytokine may be wild-type (e.g., wild-type recombinant) or genetically engineered (e.g., to introduce one or more mutations). In some embodiments, the cytokine is an engineered cytokine, e.g., an engineered interleukin. In some embodiments, the engineered cytokine comprises one or more mutations, e.g., to afford different biological properties relative to the wild type variant. For example, the cytokine molecule may be an engineered ‘decoy-resistant’ interleukin (e.g., decoy-resistant IL-18), as described by Zhou et al. (Nature (2020) 583:609-614; incorporated herein by reference in its entirety). In some embodiments, the cytokine is an engineered interleukin (e.g., engineered IL-2, or engineered IL-18).

In some embodiments, the cytokine is engineered to improve one or more pharmacokinetic properties. In some embodiments, the engineered cytokine comprises a cytokine fused to another molecule (e.g., an antibody), e.g., to increase the serum half-life of the cytokine. In some embodiments, the cytokine is fused to a long-half-life protein or protein domain (e.g., Fc fusion, transferrin [Tf] fusion, or albumin fusion). In some embodiments, the cytokine is fused to an inert polypeptide (e.g., XTEN or recombinant PEG (rPEG); a homo-amino acid polymer (HAP; e.g., by HAPylation); a proline-alanine-serine polymer (PAS; e.g., by PASylation); or an elastin-like peptide (ELP; e.g., by ELPylation)). In some embodiments, the cytokine is conjugated to repeat chemical moieties (e.g., a polymer, e.g., PEG by PEGylation; or hyaluronic acid), e.g., to increase the hydrodynamic radius of the cytokine. In some embodiments, the negative charge of the cytokine is increased, e.g., by polysialylation of the cytokine, or by fusing a negatively charged, highly sialylated peptide (e.g., carboxy-terminal peptide [CTP; of chorionic gonadotropin (CG) β-chain]) to the cytokine. In some embodiments, the cytokine is non-covalently bound to a long-half-life protein, e.g., HSA, human IgG, or transferrin. In some embodiments, the cytokine is chemically conjugated to long-half-life proteins, e.g., a human IgG, an Fc moietie, or HSA. Methods for preparing and using fusion proteins, and similarly modified proteins, e.g., for half-life extension, has been described (see, e.g., Strohl et al. BioDrugs (2015) 29:215-239; and references cited therein).

Engineered cytokines (e.g., engineered interleukins) may be produced by known methods, e.g., by a directed evolution technique (see, e.g., Zhou et al., vide supra). Examples of engineered cytokines (e.g., engineered interleukins) have been described (see, e.g., WO 2012/107417; WO 2009/061853; U.S. Pat. No. 9,580,486; Minsahwi et al. Front Immunol. 2020 (11); 1794; Tang et al. Cytokine X (2019) 100001; Mitra et al. Immunity (2015) 42:826-838; Casadesds et al. Oncolmmunology (2020) 9: 1770565; Zhou et al, vide supra; each of which are incorporated herein by reference in their entirety).

Without wishing to be bound by theory, engineered cytokines (e.g., ‘decoy-resistant’ interleukins, e.g., decoy resistant IL-18) can have beneficial properties compared to their wild-type counterparts. By way of example, decoy-resistant IL-18 can maintain signaling potential but remain impervious to inhibition by IL-18 binding protein (IL-18BP). In some embodiments, the engineered cytokine has improved stability compared to the wild-type cytokine (e.g., improved temperature-dependent stability, pH-dependent stability, or both). In some embodiments, the engineered cytokine has improved serum half-life, relative to the wild-type cytokine. In some embodiments, the engineered cytokine has modified affinity (e.g., enhanced affinity) for a receptor, different to the wild-type cytokine. In some embodiments, the engineered cytokine has lower toxicity, relative to the wild-type cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1α, IL-1β, IL-2, IL-3, IL-4, IL-5, IL-6, IL-7, IL-8, IL-10, IL-12, IL-18, IL-21, IFN-α, IFN-β, IFN-γ, MIP-1α, MIP-1β, TGF-β, TNF-α, and TNFβ. In one embodiment the cytokine of the multispecific or multifunctional polypeptide is a cytokine selected from the group of GM-CSF, IL-2, IL-7, IL-8, IL-10, IL-12, IL-15, IL-18, IL-21, IFN-α, IFN-γ, MIP-1α, MIP-1β and TGF-β. In one embodiment the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In one embodiment the cytokine of the multispecific or multifunctional polypeptide is a cytokine selected from the group of IL-2, IL-7, IL-10, IL-12, IL-15, IL-18, IFN-α, and IFN-γ. In one embodiment the cytokine of the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation can increase homogeneity of the product obtainable in recombinant production.

In one embodiment, the cytokine is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

In another embodiment the cytokine is IL-12. In a specific embodiment said IL-12 cytokine is a single chain IL-12 cytokine. In one embodiment, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine is IL-10. In another specific embodiment the IL-10 cytokine is a monomeric IL-10 cytokine. In one embodiment, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation.

In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the α-subunit of the IL-15 receptor has a reduced ability to bind to fibroblasts throughout the body, resulting in improved pharmacokinetics and toxicity profile, compared to a wild-type IL-15 polypeptide. In one embodiment, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

In another embodiment the cytokine is interleukin-18 (IL-18). In one embodiment, the cytokine is an IL-18 variant polypeptide. In one embodiment said IL-18 cytokine is a decoy-resistant IL-18 (see, e.g., U.S. Patent Publication No.: 2019/0070262; and Zhou et al. vide supra, each of which are incorporated herein by reference in their entirety). In one embodiment, the IL-18 cytokine can elicit one or more of the cellular responses selected from the group consisting of: proliferation in a NK cell, differentiation in a NK cell, proliferation in a T cell, differentiation in a T cell, and simulation of lymphocytes (e.g., innate lymphocytes). Without wishing to be bound by theory, IL-18 can be used as an effective immunotherapeutic, and is well tolerated in humans (see, e.g., Robertson et al. Clin. Cancer Res. (2006) 12:4265-4273). In some embodiments, IL-18 (e.g., decoy-resistant IL-18) increases the population of precursor T cells (e.g., CD8 T cells) that express a transcription factor (e.g., Tcf1) with anti-tumor function. In some embodiments, IL-18 (e.g., decoy-resistant IL-18) promotes the differentiation of T-cells towards a highly active polyfunctional effector phenotype. In some embodiments, IL-18 (e.g., decoy-resistant IL-18) decreases the prevalence of exhausted CD8+ T cells (e.g., those that express the transcriptional regulator of exhaustion TOX). In some embodiments, IL-18 (e.g., decoy-resistant IL-18) enhances the activity and/or maturation of NK cells.

In some embodiments, the cytokine is a decoy-resistant interleukin IL-18. In some embodiments, the cytokine is an IL-18 variant polypeptide, wherein the IL-18 variant polypeptide specifically binds to IL-18 receptor (IL-18R) and wherein, compared to wild type (WT) IL-18, the IL-18 variant polypeptide comprises at least one mutation and exhibits substantially reduced binding to IL-18 binding protein (IL-18BP). In some embodiments, the cytokine is an IL-18 variant polypeptide comprising at least one mutation selected from the group consisting of Y1X, L5X, K8X, M51X, K53X, S55X, Q56X, P57X, G59X, M60X, E77X, Q103X, S105X, D110X, N111X, M113X, V153X, and N155X, relative to a wild-type IL-18 sequence, e.g., SEQ ID NO: 30 of US2019/0070262. In some embodiments, the cytokine is an IL-18 variant polypeptide comprising at least one mutation selected from the group consisting of Y1H, Y1R, L5H, L51, L5Y, K8Q, K8R, M51T, M51K, M51D, M51N, M51E, M51R, K53R, K53G, K53S, K53T, S55K, S55R, Q56E, Q56A, Q56R, Q56V, Q56G, Q56K, Q56L, P57L, P57G, P57A, P57K, G59T, G59A, M60K, M60Q, M60R, M60L, E77D, Q103E, Q103K, Q103P, Q103A, Q103R, S105R, S105D, S105K, S105N, S105A, D110H, D110K, D110N, D110Q, D110E, D11S, D110G, N111H, N111Y, N111D, N111R, N111S, N111G, M113V, M113R, M113T, M113K, V153I, V153T, V153A, N155K, and N155H, relative to a wild-type IL-18 sequence, e.g., SEQ ID NO: 30 of US2019/0070262. In some embodiments, the cytokine is an IL-18 variant polypeptide comprising at least 6 mutations selected from: Y1X, L5X, K8X, M51X, K53X, 555X, Q56X, P57X, G59X, M60X, E77X, Q103X, S105X, D110X, N111X, M113X, V153X, and N155X, relative to a wild-type IL-18 sequence, e.g., SEQ ID NO: 30 of US2019/0070262. In some embodiments, the cytokine is an IL-18 variant polypeptide comprising mutations at positions M51, K53, Q56 D110, and N111, relative to a wild-type IL-18 sequence, e.g., SEQ ID NO: 30 of US2019/0070262. In some embodiments, the cytokine is an IL-18 variant polypeptide sequence comprising the following five mutations: (i) M51E, M51R, M51K, M51T, M51D, or M51N; (ii) K53G, K53S, K53T, or K53R; (iii) Q56G, Q56R, Q56L, Q56E, Q56A, Q56V, or Q56K; (iv) D10S, D110N, D110G, D110K, D110H, D110Q, or D110E; and (v) N111G, N111R, N111S, N111D, N111H, or N111Y; relative to a wild-type IL-18 sequence, e.g., SEQ ID NO: 30 of US2019/0070262. In some embodiments, the cytokine is an IL-18 variant polypeptide further comprising mutations at positions P57 and M60, relative to a wild-type IL-18 sequence, e.g., SEQ ID NO: 30 of US2019/0070262. In some embodiments, the cytokine is an IL-18 variant polypeptide comprising the following seven mutations: (i) M51E, M51R, M51K, M51T, M51D, or M51N; (ii) K53G, K53S, K53T, or K53R; (iii) Q56G, Q56R, Q56L, Q56E, Q56A, Q56V, or Q56K; (iv) D11S, D110N, D110G, D110K, D110H, D110Q, or D110E; (v) N111G, N111R, N111S, N111D, N111H, or N111Y; (vi) P57A, P57L, P57G, or P57K; and (vii) M60L, M60R, M60K, or M600; relative to a wild-type IL-18 sequence, e.g., SEQ ID NO: 30 of US2019/0070262. In some embodiments, the cytokine is an IL-18 variant polypeptide sequence comprising a polypeptide sequence described US2019/0070262 (e.g., one of SEQ ID NOs.: 34-59, 60-72, 73-91, 191-193, or a fragment thereof).

Without wishing to be bound by theory, an IL-18 variant protein (e.g., a decoy-resistant IL-18) can avoid potential attenuation of activity due to the presence of IL-18 binding proteins, such as IL-18BP. Without wishing to be bound by theory, an IL-18 variant protein (e.g., decoy-resistant IL-18) can evade IL-18BP, and can further promote innate anti-tumor immunity by stimulating NK cell activity and/or enhancing NK cell maturation, and can exert anti-tumor immunity against tumors that are resistant to canonical immune checkpoint blockade therapy (e.g., anti-PD-1 plus anti-CLTA-4) due to loss of MHC class I surface expression.

Mutant cytokine molecules useful as effector moieties can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In one embodiment, the cytokine is GM-CSF. In a specific embodiment, the GM-CSF cytokine can elicit proliferation and/or differentiation in a granulocyte, a monocyte or a dendritic cell.

In one embodiment, the cytokine is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In one embodiment the cytokine, particularly a single-chain cytokine, is IFNγ. In a specific embodiment, the IFN-γ cytokine can elicit one or more of the cellular responses selected from the group of: increased macrophage activity, increased expression of MHC molecules, and increased NK cell activity.

In one embodiment the cytokine, particularly a single-chain cytokine, is IL-7. In a specific embodiment, the IL-7 cytokine can elicit proliferation of T and/or B lymphocytes.

In one embodiment, the cytokine is IL-8. In a specific embodiment, the IL-8 cytokine can elicit chemotaxis in neutrophils. In one embodiment, the cytokine, particularly a single-chain cytokine, is MIP-1α. In a specific embodiment, the MIP-1α cytokine can elicit chemotaxis in monocytes and T lymphocyte cells. In one embodiment, the cytokine is MIP-1β. In a specific embodiment, the MIP-1β cytokine can elicit chemotaxis in monocytes and T lymphocyte cells. In one embodiment, the cytokine is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In some embodiments, the microneedle or combination treatment disclosed herein includes a cytokine. In embodiments, the cytokine includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

Other immunomodulatory agents include, but are not limited to cancer vaccines, such as a viral cancer therapeutic agent and/or a cancer vaccine which comprises a tumor antigen, such as a neoantigen.

### Active Agent Formulations

At least one therapeutic agent disclosed herein can be incorporated into a variety of formulations, compositions, articles, devices, and/or preparations for administration, e.g., to achieve controlled- and/or sustained release. More particularly, at least one therapeutic agent can be formulated into formulations, compositions, articles, devices, and/or preparations by combination with appropriate, pharmaceutically acceptable carriers or diluents, and can be formulated into preparations in semi-solid, solid, or liquid formats. In some embodiments, the formulations, compositions, articles, devices, and/or preparations described herein comprise silk fibroin. Exemplary formulations, compositions, articles, devices, and/or preparations comprise: a microneedle (e.g., a microneedle device, e.g., a microneedle patch, e.g., as described herein), an implantable device (e.g., a pump, e.g., a subcutaneous pump), an injectable formulation, a depot, a gel (e.g., a hydrogel), an implant, and a particle (e.g., a microparticle and/or a nanoparticle). As such, administration of the compositions can be achieved in various ways, including intradermal, intramuscular, transdermal, subcutaneous, or intravenous administration. Moreover, the formulations, compositions, articles, devices, and/or preparations can be formulated and/or administered to achieve controlled- and/or sustained release of the therapeutic agent.

In some embodiments, the therapeutic agent is administered, e.g., substantially sustained, over a period of, or at least 1, 5, 10, 15, 30, 45 minutes; a period of, or at least, 1, 2, 3, 4, 5, 10, 24 hours; a period of, or at least, 1, 2, 3, 4, 5, 6, 7, 8, 9, 10, 11, 12, 13, 14 days; a period of, or at least, 1, 2, 3, 4, 5, 6, 7, 8 weeks; a period of, or at least 1, 2, 3, 4, 5, 6, 7, 8, 9, 10, 11 months; a period of, or at least, 1, 2, 3, 4, 5 years, or longer. In one embodiment, a therapeutic agent, such as an anti-cancer agent, an immunomodulatory agent, or a combination thereof, is administered as a controlled- or sustained release formulation, dosage form or device. In one embodiment, a therapeutic agent, such as an anti-cancer agent, an immunomodulatory agent, or a combination thereof, is administered as a burst release formulation, dosage form or device. In certain embodiments, the therapeutic agent is formulated for continuous delivery, e.g., intradermal, intramuscular, and/or intravenous continuous delivery. In some embodiments, the composition or device for the controlled- or sustained-release of the therapeutic agent is chosen from: a microneedle (e.g., a microneedle device, e.g., a microneedle patch), an implantable device (e.g., a pump, e.g., a subcutaneous pump), an injectable formulation, a depot, a gel (e.g., a hydrogel), an implant, or a particle (e.g., a microparticle and/or a nanoparticle). In one embodiment, the therapeutic agent is in a silk fibroin-based microneedle controlled- or extended release dosage form or formulation (e.g., a microneedle described herein). In one embodiment, the therapeutic agent is administered via an implantable device, e.g., a pump (e.g., a subcutaneous pump), an implant, an implantable tip of a microneedle, or a depot. The delivery method can be optimized such that a therapeutic agent dose as described herein (e.g., a standard dose) is administered and/or maintained in the subject for a pre-determined period (e.g., a period of, or at least: 1, 5, 10, 15, 30, 45 minutes; 1, 2, 3, 4, 5, 10, 24 hours; 1, 2, 3, 4, 5, 6, 7, 8, 9, 10, 11, 12, 13, 14 days; 1, 2, 3, 4, 5, 6, 7, 8 weeks; 1, 2, 3, 4, 5, 6, 7, 8, 9, 10, 11 months; 1, 2, 3, 4, 5 years, or longer). The substantially sustained or extended release of the therapeutic agent can be used for prevention or treatment of a disease and/or a disorder, such as cancer, for a period of hours, days, weeks, months, or years.

The present disclosure provides, in some embodiments, formulations, compositions, articles, devices, and/or preparations that can be formulated and/or configured for controlled- or sustained-release of a therapeutic agent in an amount (e.g., a dosage) and/or over a time period sufficient to result in an immune response (e.g., a cellular immune response and/or a humoral immune response) to an antigen (e.g., a tumor-specific antigen, such as a neoantigen) in the subject.

In some embodiments, the formulations, compositions, articles, devices, and/or preparations of the present disclosure can be formulated and/or configured for controlled- or sustained-release of a at least therapeutic agent in an amount (e.g., a dosage) and/or over a time period sufficient to result in an immunity (e.g., cancer immunity) in the subject.

The substantially continuously or extended release delivery or formulation of the therapeutic agent can be used for prevention or treatment of a disease or disorder such as a cancer for a period of hours, days, weeks, months, or years.

In some embodiments, a therapeutic agent described herein can be added to the silk fibroin solution, e.g., before forming the silk fibroin microneedles or microneedle devices described herein. In embodiments, a silk fibroin solution can be mixed with therapeutic agent, and then used in the fabrication of an implantable microneedle tip, e.g., by the process of filling and/or casting, drying, and/or annealing to produce a microneedle having any of the desired material properties, as described herein.

Without being bound by theory, the ratio of silk fibroin to therapeutic agent in a silk fibroin tip, e.g., an implantable tip, of a microneedle influences their release. In some embodiments, increased silk concentration in the tip favors a slower release and/or greater therapeutic agent retention within the tip. Any concentration of silk may be used, as long as the concentration allows for printing and has the mechanical strength sufficient to pierce the skin.

In some embodiments, silk fibroin can be used at a concentration ranging from about 1% w/v to about 10% w/v (e.g., about 1, 2, 3, 4, 5, 6, 7, 8, 9, or 10% w/v) in the fabrication of a microneedle, or a component thereof, as described herein. In some embodiments, silk fibroin can be used at a concentration ranging from about 1% w/v to about 30% w/v (e.g., about 1, 2, 3, 4, 5, 6, 7, 8, 9, 10, 15, 20, 25, or 30% w/v) in the fabrication of a microneedle, or a component thereof, as described herein.

In some embodiments, a silk fibroin can be used in an amount of about 0.5 μg to about 500 μg silk fibroin in the fabrication of a microneedle, or a component thereof, as described herein. In some embodiments, a silk fibroin can be used in an amount of about 0.5 μg to about 5 μg, or about 1 μg to about 10 μg, or about 5 μg to about 15 μg, or about 10 μg to about 20 μg, or about 15 μg to about 25 μg, or about 20 μg to about 30 μg, or about 25 μg to about 35 μg, or about 30 μg to about 40 μg, or about 35 μg to about 45 μg, or about 40 μg to about 50 μg, or about 45 μg to about 55 μg, or about 50 μg to about 60 μg, or about 55 μg to about 65 μg, or about 60 μg to about 70 μg, or about 65 μg to about 75 μg, or about 70 μg to about 80 μg, or about 75 μg to about 85 μg, or about 80 μg to about 90 μg, or about 85 μg to about 95 μg, or about 90 μg to about 100 μg, or about 95 μg to about 150 μg, or about 125 μg to about 175 μg, or about 150 μg to about 200 μg, or about 225 μg to about 275 μg, or about 250 μg to about 300 μg, or about 325 μg to about 375 μg, or about 350 μg to about 400 μg, or about 425 μg to about 475 μg, or about 450 μg to about 500 μg silk fibroin in the fabrication of a microneedle, or a component thereof, as described herein. In some embodiments, a silk fibroin can be used in an amount of at least about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin in the fabrication of a microneedle, or a component thereof, as described herein. In some embodiments, a silk fibroin can be used in an amount of at most about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin in the fabrication of a microneedle, or a component thereof, as described herein. In some embodiments, a silk fibroin can be used in an amount of about 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, or 500 μg silk fibroin in the fabrication of a microneedle, or a component thereof, as described herein. In some embodiments, a silk fibroin can be used in an amount of about 2.42 ug to 242 ug in the fabrication of a microneedle, or a component thereof, as described herein.

In some embodiments, a silk fibroin can be used in an amount of about 1% to about 75%, about 1% to about 5%, about 10% to about 60%, about 15% to about 50%, or about 20% to about 40% by weight, of silk fibroin in the fabrication of a microneedle, or a component thereof, as described herein. In some embodiments, a silk fibroin can be used in an amount of at least about 1, 2, 3, 4, 5, 6, 7, 8, 9, 10, 15, 20, 25, 30, 35, 40, 45, 50, 55, 60, 65, 70, or 75% by weight, of silk fibroin in the fabrication of a microneedle, or a component thereof, as described herein. In some embodiments, a silk fibroin can be used in an amount of at most about 1, 2, 3, 4, 5, 6, 7, 8, 9, 10, 15, 20, 25, 30, 35, 40, 45, 50, 55, 60, 65, 70, or 75% by weight, of silk fibroin in the fabrication of a microneedle, or a component thereof, as described herein. In some embodiments, a silk fibroin can be used in an amount of about 1, 2, 3, 4, 5, 6, 7, 8, 9, 10, 15, 20, 25, 30, 35, 40, 45, 50, 55, 60, 65, 70, or 75% by weight, of silk fibroin in the fabrication of a microneedle, or a component thereof, as described herein.

Exemplary Excipients

In addition, the formulations, compositions, articles, devices, and/or preparations can be formulated with common excipients, diluents or carriers for administered by the intradermal, intramuscular, transdermal, subcutaneous, or intravenous routes. In some embodiments, the formulations, compositions, articles, devices, and/or preparations can be administered, e.g., transdermally, and can be formulated as controlled- or sustained-release dosage forms and the like. The formulations, compositions, articles, devices, and/or preparations described herein can be administered alone, in combination with each other, or they can be used in combination with other known therapeutic agents.

Suitable formulations for use in the present disclosure are found in Remington's Pharmaceutical Sciences (1985). Moreover, for a review of methods for drug delivery, see, Langer (1990) Science 249:1527-1533. The formulations, compositions, articles, devices, and/or preparations described herein can be manufactured in a manner that is known to those of skill in the art, e.g., by mixing, dissolving, granulating, dragee-making, levigating, emulsifying, encapsulating, entrapping or lyophilizing processes. The following methods and excipients are merely exemplary and are in no way limiting.

The silk fibroin formulations used in the fabrication of the microneedles described herein may include excipients. In embodiments, inclusion of an excipient may be for the purposes of improving the stability of an incorporated therapeutic agent, such as an anti-cancer agent, an immunomodulatory agent, or a combination thereof; to increase silk matrix porosity and diffusivity of the therapeutic agent, such as an anti-cancer agent, an immunomodulatory agent, or a combination thereof, from the formulation, composition, article, device, preparation, and/or microneedle, e.g., microneedle tip; and/or to increase crystallinity/beta-sheet content of silk matrix to render the silk-material insoluble.

Exemplary excipients include, but are not limited to, a sugar or a sugar alcohol (e.g., sucrose, trehalose, sorbitol, mannitol, or a combination thereof), a divalent cation (e.g., Ca²⁺, Mg²⁺, Mn²⁺, and Cu²⁺), a surfactant (e.g., an octyl phenol ethoxylate (e.g., Triton-X), a polysorbate, a poloxamer, and/or a polyethoxylated alcohol), a polyol (e.g., glycerol), a glycol (e.g. propylene glycol, PEG) and/or a buffer. In some embodiments, the concentration of an excipient can be used to modify the porosity of the matrix, e.g., with sucrose being used as the most common excipient for this purpose. Excipients may also be added to favor silk self-assembly into ordered beta-sheet secondary structure, and such excipients generally can participate in hydrogen bonding or charge interactions with silk to achieve this effect. Non-limiting examples of excipients that can be used to favor silk self-assembly into ordered beta-sheet secondary structure include monosodium glutamate (e.g., L-glutamic acid), lysine, sugar alcohols (e.g., sorbitol and/or glycerol), and solvents (e.g., DMSO, methanol, and/or ethanol).

In some embodiments, the sugar or the sugar alcohol is sucrose present in an amount less than 70% (w/v), less than 60% (w/v), less than 50% (w/v), less than 40% (w/v), less than 30% (w/v), less than 20% (w/v), less than 10% (w/v), less than 9% (w/v), less than 8% (w/v), less than 7% (w/v), less than 6% (w/v), or 5% (w/v) or less, e.g., immediately before drying.

In some embodiments, the sugar or the sugar alcohol is sucrose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the sugar or the sugar alcohol is trehalose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the sugar or the sugar alcohol is sorbitol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the sugar or the sugar alcohol is glycerol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the surfactant (e.g., an octyl phenol ethoxylate (e.g., Triton-X), a polysorbate, a poloxamer, and/or a polyethoxylated alcohol) is present in an amount between about 0.005% (w/v) to about 1% (w/v), about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the polyol (e.g., glycerol) is present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the glycol (e.g. propylene glycol, e.g., PEG) is present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5% to about 5%, or about 2.4% (w/v), about 2.5%, or about 5% (w/v), e.g., immediately before drying.

In some embodiments, the therapeutic agent preparation further comprises a divalent cation. In some embodiments, the divalent cation is selected from the group consisting of Ca²⁺, Mg²⁺, Mn²⁺, and Cu²⁺. In some embodiments, the divalent cation is present in the preparation, e.g., immediately before drying, in an amount between 0.1 mM and 100 mM. In some embodiments, the divalent cation is present in the preparation, e.g., immediately before drying, in an amount between 10⁻⁷ and 10⁻⁴ moles per standard dose of the therapeutic agent (e.g., an anti-cancer agent, an immunomodulatory agent, a viral immunogen, or a combination thereof). In some embodiments, the divalent cation is present in the preparation immediately before drying in an amount between 10⁻¹⁰ to 2×10⁻³ moles.

In some embodiments, the therapeutic agent further comprises poly(lactic-co-glycolic acid) (PGLA).

In some embodiments, the therapeutic agent preparation further comprises a buffer, e.g., immediately before drying. In some embodiments, the buffer has buffering capacity between pH 3 and pH 8, between pH 4 and pH 7.5, or between pH 5 and pH 7. In some embodiments, the buffer is selected from the group consisting of PBS, HEPES, and a CP buffer. In some embodiments, the buffer is present in the preparation, e.g., immediately before drying, in an amount between 0.1 mM and 100 mM. In some embodiments, the buffer is present in an amount between 10⁻⁷ and 10⁻⁴ moles per standard dose of the therapeutic agent (e.g., an anti-cancer agent, an immunomodulatory agent, a viral immunogen, or a combination thereof). In some embodiments, the buffer is present in an amount between 10⁻¹⁰ to 2×10⁻³ moles.

In addition, the therapeutic agent can also be formulated as a depot, gel, or hydrogel preparation. Such long acting formulations can be administered by implantation (for example subcutaneously or intramuscularly) or by intramuscular injection. Thus, for example, the therapeutic agent can be formulated with suitable polymeric or hydrophobic materials (for example as an emulsion in an acceptable oil) or ion exchange resins, or as sparingly soluble derivatives, for example, as a sparingly soluble salt.

In one embodiment, the therapeutic agent is administered via an implantable infusion device, e.g., a pump (e.g., a subcutaneous pump), an implant or a depot. Implantable infusion devices typically include a housing containing a liquid reservoir which can be filled transcutaneously by a hypodermic needle penetrating a fill port septum. The medication reservoir is generally coupled via an internal flow path to a device outlet port for delivering the liquid through a catheter to a patient body site. Typical infusion devices also include a controller and a fluid transfer mechanism, such as a pump or a valve, for moving the liquid from the reservoir through the internal flow path to the device's outlet port.

In some embodiments, the therapeutic agent can be packaged and/or formulated as a particle, e.g., a microparticle and/or a nanoparticle. Typically nanoparticles are from 10, 15, 20, 25, 30, 35, 45, 50, 75, 100, 150 or 200 nm, or between 200 nm and 1,000 nm, e.g., 10, 15, 20, 25, 30, 35, 45, 50, 75, 100, 150, or 200, or 20 or 30 or 50-400 nm in diameter. Smaller particles tend to be cleared more rapidly from the system. Therapeutic agents, including therapeutic agents described herein, can be entrapped within or coupled, e.g., covalent coupled, or otherwise adhered, to nanoparticles.

Lipid- or oil-based nanoparticles, such as liposomes and solid lipid nanoparticles, can be used to deliver therapeutic agents, such as an anti-cancer agent, an immunomodulatory agent, or a combination thereof, as described herein. Solid lipid nanoparticles for the delivery of therapeutic agents are described (see, e.g., Serpe et al. (2004) Eur. J. Pharm. Bioparm. 58:673-680, and Lu et al. (2006 Eur. J. Pharm. Sci. 28: 86-95). Polymer-based nanoparticles, e.g., PLGA-based nanoparticles can be used to deliver agents described herein. These tend to rely on biodegradable backbone with the therapeutic agent intercalated (with or without covalent linkage to the polymer) in a matrix of polymer. PLGA is widely used in polymeric nanoparticles, (see, e.g., Hu et al. (2009) J. Control. Release 134:55-61; Cheng et al. (2007) Biomaterials 28:869-876, and Chan et al. (2009) Biomaterials 30:1627-1634). PEGylated PLGA-based nanoparticles can also be used to deliver therapeutic agents, (see, e.g., Danhhier et al., (2009) J. Control. Release 133:11-17, Gryparis et al (2007) Eur. J. Pharm. Biopharm. 67:1-8). Metal-based nanoparticles, e.g., gold-based nanoparticles, can also be used to deliver therapeutic agents. Protein-based nanoparticles, e.g., albumin-based nanoparticles, can be used to deliver therapeutic agents described herein. In some embodiments, a therapeutic agent can be bound to nanoparticles of human albumin.

A broad range of nanoparticles are known in the art. Exemplary approaches include those described in WO2010/005726, WO2010/005723, WO2010/005721, WO2008/121949, WO2010/075072, WO2010/068866, WO2010/005740, WO2006/014626, U.S. Pat. Nos. 7,820,788, and 7,780,984, the contents of which are incorporated herein in reference by their entirety.

### Dosages

Any dosage amount (e.g., a standard dose and/or a fractional dose) of a therapeutic agent that is capable of eliciting a treatment response (e.g., an anti-cancer response, e.g., an immune response) in a subject when administered by a microneedle of the present disclosure, may be used according to the methods described herein.

Without wishing to be bound by theory, the total dosage amount (e.g., a standard dose) of a therapeutic agent to be administered by a microneedle described herein can be divided between a plurality of microneedles (e.g., within a patch), such that a microneedle tip can comprises less than about 1% of the total dosage amount (e.g., in an array comprising about 121 microneedles), or at least about 1%, 2%, 3%, 4%, 5%, 6%, 7%, 8%, 9%, 10%, 11%, 12%, 13%, 14%, 15%, 16%, 17%, 18%, 19%,20%,21%,22%,23%,24%, or 25% or more of the total dosage amount.

In some embodiments, the therapeutic agent dosage amount loaded into a microneedle patch can be manipulated via the concentration of therapeutic agent in the formulated solution that forms the needle tips, the volume of solution dispensed into each needle tip, and the total number of needles. In some embodiments, the former two are more convenient means of varying dose. The dosage released into the subject is related to deployment efficiency (the portion of needle tips that are left behind in the biological barrier, such as the skin, after the patch is removed), and also the release profile over time and the residence time of the tips within the subject. Because of the continuous sloughing of skin from the epidermis, deeper deployment within the skin is related to longer residence time. Therefore, it is desirable to maximize the penetration depth of the needle tip (up to a limit defined by the depth of pain receptors within the skin, e.g., at a depth of between about 100 μm and about 600 μm), and also to have the antigen spatially concentrated toward the tip of the needle.

The formulations, compositions, articles, devices, and/or preparations described herein, including the silk fibroin tip formulation, are designed to not only release, e.g., sustain the release, of a therapeutic agent over the duration of tip retention in the dermis, but to also maintain stability of the therapeutic agent during this period of time (e.g., at least about 1-2 weeks). In some embodiments, approximately 95-100% of the total dosage amount incorporated, e.g., in a formulation, composition, article, device, preparation, and/or a microneedle described herein, can be expected to be available for delivery, e.g., into a subject, e.g., into a tissue of a subject, such as the skin, tumor, a mucous membrane, an organ tissue, a buccal cavity, a tissue, or a cell membrane. Without being bound by theory, successful deployment of a microneedle into the skin is at least about 50% and can be as high as 100% of an array (e.g., upon application at least about 50%, 60%, 70%, 80%, 90% or more (e.g., 100%) of the total number of microneedles of an array are successfully deployed within, e.g., the skin, for controlled- or sustained-release of a therapeutic agent, such as an anti-cancer agent, an immunomodulatory agent, or a combination thereof). In some embodiments, a portion of antigen may not be released from the silk tips during the duration of deployment.

### Methods of Fabricating a Microneedle

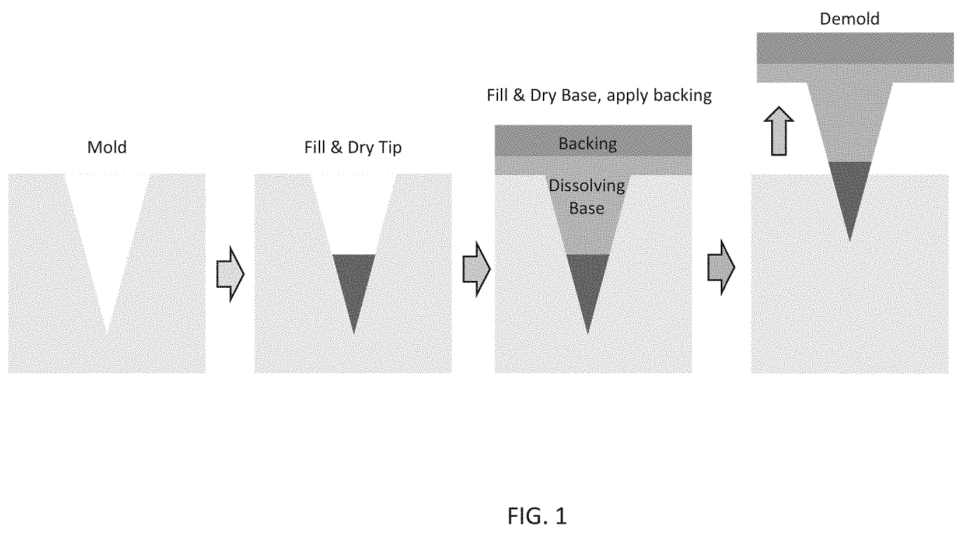

A schematic diagram depicting an exemplary method of fabrication of a microneedle of the present disclosure is shown in FIG. 1. Machine vision guided dispensing of precise nanoliter (nL) volumes of silk fibroin solution into individual needle cavities enables different dosages and formulations to be incorporated within releasable tips of a microneedle device (e.g., a microneedle array or patch). An exemplary microneedle device (e.g., a microneedle array or patch), comprises an 11×11 cone array. It should be understood that the microneedle device may include needle cavities produced in an array of varying number of cavities and orientations to achieve a desired result.

Mold Production

In some embodiments, a mold is used in the fabrication of a microneedle device. As will be discussed in greater detail below, a sterilized mold is used to produce a microneedle device having an array of releasable tips embodying a therapeutic agent-silk formulation (e.g., an anticancer agent-silk formulation, an immunomodulatory agent-silk formulation, an antigen-silk formulation, or a combination thereof).

For example, a silicone (DOW Corning Sylgard® 184) resin may be cast against a positive master having the intended geometry of a microneedle array. Once the silicone has cured, it may be removed from the master. The master can then be reused for a large number of silicone castings. Throughout the fabrication process the silicone mold may be inspected for defects (e.g., between castings). If desired, the silicone mold can be sterilized, for example, by autoclaving. In one embodiment, the mold includes a mold body having an array of needle cavities formed within the mold body.

In some embodiments, other types of silicone and/or other materials and processes may be used to fabricate the mold. For example, liquid silicone injection molding and thermoplastic elastomer injection molding may be used. Without wishing to be bound by theory, it may be understood that a key requirement is that the mold material be soft and flexible (e.g., comprise a Shore hardness of about 50A) and have low adhesion with silk and other materials used in the construction of the patch.

Tip Filling

Tip formulation consisting of silk fibroin, therapeutic agent (e.g., anti-cancer agent, immunomodulatory agent, antigen, or combination thereof) and potentially other excipients in aqueous solution, is dispensed into each needle cavity in the mold via nanoliter printing.

Currently this is done at lab scale using machine vision guided automated dispensing system, such as a Biojet Elite™ AD3400 dispensing system produced by BioDot, but systems with similar capabilities made by other suppliers can be employed. In some embodiments, the working volume of the dispenser used for tip filling (e.g., a BioDot™ dispenser) is enclosed and is maintained at an elevated relative humidity (RH), e.g., an RH of 60% or greater, e.g., to slow drying of the formulation and/or to avoid buildup of dry solids on the dispensing nozzle. In an embodiment, the working volume of the BioDot™ dispenser is enclosed and is maintained at 60% relative humidity (RH) to slow drying of the formulation and avoid buildup of dry solids on the dispensing nozzle.

Molds are placed within a fixture that constrains their locations on the processing platform of the BioDot™ dispenser. The machine uses a camera to image each mold and a machine vision algorithm identifies the precise location and orientation of the array of needle cavities in each mold. This location is used to direct the subsequent dispensing steps. The filled molds are inspected using a stereomicroscope for filling defects such as misaligned dispenses or large bubbles in the liquid.

Primary Drying

In some embodiments, the filled molds are set aside to dry, e.g., within an enclosure that maintains a desired ambient humidity. In an embodiment, the filled molds are set aside to dry within the machine enclosure for about 7 minutes. In some embodiments, the silk fibroin solubility may be modulated by manipulating the drying time. Without wishing to be bound by theory, during drying, the silk structure can shift to more beta-sheets and become less soluble (e.g., insoluble), and this effect can be increased by drying more slowly and/or by incubating at elevated humidity, e.g., from about 10% up to about 100% RH

After drying, the above dispensing process may be repeated. In some embodiments, the molds are moved to a chamber with approximately saturated humidity and incubated overnight to slowly dry the tips. During this time the silk structure can shift to more beta-sheets and becomes less soluble (e.g., insoluble) (annealing).

Secondary Drying

In some embodiments, the molds are moved to a chamber in which humidity is controlled to about 10% to about 25% relative humidity (RH) and ambient room temperature and kept overnight (about 14 hours) to complete drying. This is the “secondary” drying step.

Water Annealing

In some embodiments, the molds (e.g., molds containing dried silk fibroin tips) are transferred to a vacuum desiccator that also contains about 500 mL of deionized water (DIW). The desiccator is closed and vacuum is drawn for about 5 minutes using the main vacuum line in the lab. After 5 minutes, the outlet valve of the desiccator is closed and it is placed within an incubator holding at 37° C. for four hours. After four hours, the desiccator is vented and the molds are transferred back to the 25% RH chamber at ambient room temperature.

Post-Anneal Drying

Molds can be kept at about 10% to about 25% RH for at least four hours or up to overnight before subsequent steps.

Base Layer Filling

The dissolvable base layer can be formed by filling the mold with a base solution described herein. In some embodiments, the base solution comprises 40% w/v hydrolyzed gelatin and 10% w/v sucrose in DIW. In some embodiments, the base solution comprises 30% dextran 70 kDa, 10% sucrose, 1% glycerol, and 0.01% Triton-X100. The base layer may be filled in any suitable manner. For example, first, a volume suitable for the mold (e.g., 150 μL) of base solution can be spread evenly over the mold with a pipette. Next, the molds can be centrifuged (e.g., at 3900 rpm for up to about 2 minutes). The molds can be inspected and if any needle cavities remain unfilled, the filling and centrifuging process may be repeated. The molds can be further “topped off” with a suitable amount of base solution (e.g., 50 μL of base solution). In some embodiments, centrifuge filling can be used. In some embodiments, the base is filled in the same manner as the tips by use of a vision-guided droplet dispensing into the mold cavities.

In some embodiments, more than one base layer can be applied. For example, a first base layer may be formed according to the methods outlined above. Then, e.g., after drying the first base layer, the process may be repeated to add one or more additional base layers. The one or more additional base layers may be applied using the same base layer solution as used for the first base layer, or one or more different base layer solutions may be used as desired (e.g., one or more base layer solutions described herein). In some embodiments, the base solution is a molten liquid or a slurry. In some embodiments, the base layer is solidified using a chemical reaction (e.g., after filling).

Base Drying

The filled molds can be transferred back to the chamber at about 10% to about 25% RH and dried at least overnight and up to 3 days.

Backing Application

The patches used to generate the release, e.g., controlled- or sustained-release, of a therapeutic agent and/or improve immunogenicity (see, e.g., the Examples) had a paper backing layer; however, subsequent development has shown that adhesive plastic tape can have superior performance as a backing layer.

In some embodiments, the paper backing process is as follows: the dried base layer is partially re-wetted with 10-30 μL of DIW spread over the surface with a pipette. Whatman 903 paper is punched into 12 mm diameter circles. The circles of paper are gently pressed into the wet surface of the base layer. The wet base layer partially soaks into the paper. The molds with backing are transferred back into the 25% RH chamber to dry for at least 4 hours until use. In some embodiments, the backing (e.g., a backing containing an adhesive) is cured by light irradiation.

Adhesive Tape Process

Adhesive-backed polyester tape (e.g., 3M® Magic™ tape) is cut into a piece about 12 mm wide and about 25 mm long. One end of the tape is aligned with the patch and gently pressed onto the surface of the base layer. The free end of the tape is folder over onto itself to form a non-adhesive “handle.”

Demolding

The patches are removed from the mold before use. The flexible mold is gently bent away from the stiffer patch, and the patch is taken away from the mold. The patch is inspected for defects such as missing or broken needles.

Packaging

In the studies above, the patches were used soon after demolding and were not packaged. If extended storage is needed, assembled patches can be packaged in a container with low moisture vapor transmission rate (e.g., glass vial or thermoformed plastic tray made of low MVTR materials and a foil-backed heat-sealed lid) along with a desiccant to maintain between about 0% and about 50% (e.g., between about 0% and 10%, between about 10% and about 20%, between about 20% and about 30%, between about 30% and about 40%, or between about 40% and 50%, e.g., about 25%) relative humidity inside the package.

### Therapeutic Applications

In one aspect, the present disclosure provides methods for delivering (e.g., administering) an effective amount of a therapeutic agent, such as an anti-cancer agent, an immunomodulatory agent, or a combination thereof, across a biological barrier (e.g., a layer of skin, a cell membrane, a mucous surface, an oral cavity, a skin lesion, a tumor, or a buccal cavity).

In one aspect, the present disclosure provides methods for treating, preventing, and/or ameliorating a disease and/or a disorder in a subject, for example, a cancer and/or a skin condition in a subject. In some embodiments, delivery is by intratumoral delivery. However, in some embodiments, a microneedle may be applied to sites adjacent to a tumor (e.g., peritumoral delivery). In some embodiments, microneedle administration is as a first-line therapy, e.g., for unresectable tumors. In some embodiments, microneedle administration is as a neo-adjuvant (e.g., prior to main treatment, e.g., surgery to resect a tumor) to shrink a tumor. In some embodiments, microneedle administration is as an adjuvant (e.g., post-resection of a tumor) to lower the risk of cancer recurrence.

In one aspect, the present disclosure pertains to a method of treating a cancer in a subject. The method comprises administering to the subject a microneedle of the present disclosure such that the cancer is treated in the subject. Exemplary cancers treatable by a microneedle of the present disclosure are known in the art and disclosed herein.

In one aspect, the present disclosure pertains to a method of treating a skin condition in a subject. The method comprises administering to the subject a microneedle of the present disclosure such that the cancer is treated in the subject. Exemplary skin conditions treatable by a microneedle of the present disclosure are known in the art and disclosed herein.

In one aspect, the present disclosure pertains to a method of inducing an anti-cancer response and/or an immune response (e.g., a local and/or systemic immune response) in a subject in need thereof. In some embodiments, the anti-cancer response and/or an immune response (e.g., a local and/or systemic immune response) in a subject comprises: a decrease in tumor volume or cancer volume, a decrease in the number of tumor cells or cancer cells, a decrease in the number of metastases, an increase in life expectancy, a decrease in tumor cell proliferation or cancer cell proliferation, a decrease in tumor cell survival or cancer cell survival, a prevention of relapse, and/or amelioration of various physiological symptoms associated with the cancerous condition.

In some embodiments, the present disclosure pertains to a method of inducing an anti-cancer response and/or an immune response (e.g., a local and/or systemic immune response) in a subject in need thereof that results in at least about a 5%, 10%, 15%, 20%, 25%, 30%, 35%, 40%, 45%, 50%, 55%, 60%, 65%, 70%, 75%, 80%, 85%, 90%, 95% or 99% or more decrease in tumor volume or cancer volume in a subject, e.g., compared to a baseline value.

In some embodiments, the present disclosure pertains to a method of inducing an anti-cancer response and/or an immune response (e.g., a local and/or systemic immune response) in a subject in need thereof that results in at least about a 5%, 10%, 15%, 20%, 25%, 30%, 35%, 40%, 45%, 50%, 55%, 60%, 65%, 70%, 75%, 80%, 85%, 90%, 95% or 99% or more decrease in the number of tumor cells or cancer cells in a subject, e.g., compared to a baseline value.

In some embodiments, the present disclosure pertains to a method of inducing an anti-cancer response and/or an immune response (e.g., a local and/or systemic immune response) in a subject in need thereof that results in at least about a 5%, 10%, 15%, 20%, 25%, 30%, 35%, 40%, 45%, 50%, 55%, 60%, 65%, 70%, 75%, 80%, 85%, 90%, 95% or 99% or more decrease in the number of metastases, in a subject, e.g., compared to a baseline value.

In some embodiments, the present disclosure pertains to a method of inducing an anti-cancer response and/or an immune response (e.g., a local and/or systemic immune response) in a subject in need thereof that extends the subject's life span by at least about 15, 30, 60, 90, 120, 180 or 360 days.

In some embodiments, the present disclosure pertains to a method of inducing an anti-cancer response and/or an immune response (e.g., a local and/or systemic immune response) in a subject in need thereof that results in at least about a 5%, 10%, 15%, 20%, 25%, 30%, 35%, 40%, 45%, 50%, 55%, 60%, 65%, 70%, 75%, 80%, 85%, 90%, 95% or 99% or more decrease in tumor cell proliferation or cancer cell proliferation, e.g., compared to a baseline value.

In some embodiments, the present disclosure pertains to a method of inducing an anti-cancer response and/or an immune response (e.g., a local and/or systemic immune response) in a subject in need thereof that results in at least about a 5%, 10%, 15%, 20%, 25%, 30%, 35%, 40%, 45%, 50%, 55%, 60%, 65%, 70%, 75%, 80%, 85%, 90%, 95% or 99% or more decrease in in tumor cell survival or cancer cell survival, e.g., compared to a baseline value.

In one aspect, the present disclosure provides methods for treating a disease associated with the expression of a tumor-specific antigen (e.g., a neoantigen).

In one aspect, the present disclosure pertains to a method of inhibiting the growth of a tumor and/or a skin lesion, e.g., a tumor and/or skin lesion that expresses a tumor-specific antigen (e.g., neoantigen). In certain embodiments, a tumor to be treated can be associated with cancer-specific or tumor-specific antigens, which may not be present in normal cells. However, without wishing to be bound by theory, neoantigens can be poorly or inefficiently presented to a subject's immune system, e.g., due to the immunosuppressive tumor microenvironment (TME). In some embodiments, the methods described herein can improve the presentation of neoantigens to a subject's immune system and facilitate the development of a robust and pro-longed immune response (e.g., an anti-cancer response, e.g., a cancer immunity) in a subject. In certain embodiments, the methods described herein can result in the ablation of tumors at or near the site of initial microneedle application, and also result in the ablation of tumors at distant sites within the subject's body, e.g., wherever cancer cells expressing the neoantigen are present.

The present disclosure provides silk fibroin-based microneedles, and silk fibroin-based microneedle devices, for treating and/or inducing an immune response to a cancer (e.g., a metastatic cancer) and/or a skin condition (e.g., a skin condition). In some embodiments, the methods disclosed herein comprise contacting (e.g., administering) a microneedle device, or a plurality of microneedles, comprising an anti-cancer agent, an immunomodulatory agent, or a combination thereof, to the site of a cancer (e.g., a metastatic tumor) or a lesion (e.g., skin lesion) of a subject, thereby resulting in one or more of the following: (i) lysis of a cancerous cell, e.g., tumor cells, to release and/or to expose a cancer-associated antigen (e.g., a neoantigen) to the subject's immune system; (ii) display by an antigen presenting cell (APC) to an immune system cell (e.g., an accessory cell, such as a B-cell, a dendritic cell, and the like) of a cancer-associated antigen (e.g., the neoantigen) complexed with a major histocompatibility complex (MHC) on its surface; (iii) recognition of the displayed cancer-associated antigen (e.g., the neoantigen) by an immune effector cell, e.g., a T cell and/or an NK cell; (iv) activation and/or expansion of an immune effector cell, e.g., a T cell and/or an NK cell, specific for the displayed cancer-associated antigen (e.g., a neoantigen) in the subject; and (v) an enhanced, e.g., stimulated or up-regulated, immune response of an immune effector cells, e.g., a T cell and/or an NK cell, that promotes the killing of and/or the inhibition of the growth or the proliferation of a target cell expressing the cancer-associated antigen (e.g., a neoantigen) in the subject.

In one aspect, the present disclosure pertains to a method of releasing and/or exposing a tumor-specific antigen (e.g., neoantigen) to a subject's immune system to illicit an anti-cancer response and/or an immune response.

In one aspect, the present disclosure pertains to a method of inducing a cancer immunity in a subject, optionally wherein the cancer immunity is to a tumor-specific antigen (e.g., neoantigen).

In some embodiments of any of the methods described herein, administration of a microneedle results in the activation of an immune cell in response to a tumor-specific antigen (e.g., a neoantigen) which is exposed and/or released from a tumor as a result of treatment with the microneedle (e.g., a microneedle comprising an anti-cancer agent, and immunomodulatory agent, and/or a combination thereof). Further, wherein the activated immune cell targets a cancer cell expressing the tumor-specific antigen (e.g., the neoantigen) and thereby inhibits the growth and/or proliferation of the cancer. Optionally, wherein the targeted cancer cell is at or near the site of microneedle administration. Optionally, wherein the targeted cancer cell is located at a distant site.

In one aspect, the present disclosure pertains to a method of inducing an anti-cancer response in a subject. In some embodiments, the method comprises administering to the subject a microneedle of the present disclosure such that the immune response, e.g., to a disease associated with a tumor-specific antigen (e.g., neoantigen) expression, is induced. In various aspects of any of the methods described herein, the immunity (e.g., cancer immunity) persists in the subject for a period of time after administration of the microneedle. For example, the immunity (e.g., cancer immunity) can persist in the subject for about one week, about two weeks, about three weeks, about one month, about two months, about three months, about four months, about five months, about six months, about seven months, about eight months, about nine months, about ten months, about eleven months, about twelve months, about thirteen months, about fourteen month, about fifteen months, about sixteen months, about seventeen months, about eighteen months, about nineteen months, about twenty months, about twenty-one months, about twenty-two months, about twenty-three months, about two years, about three years, about four years, or about five years after administration of the microneedle to the subject.

In one aspect, provided herein are methods for delivering (e.g., administering) an effective amount of a therapeutic agent, such as an anti-cancer agent, an immunomodulatory agent, or a combination thereof, to the site of a tumor after tumor resection, e.g., to induce and immune response to the tumor and/or to ablate any cancer cells left behind after resection.

In one aspect, provided herein are methods for delivering (e.g., administering) an effective amount of a therapeutic agent, such as an anti-cancer agent, an immunomodulatory agent, or a combination thereof, to the site of a tumor before tumor resection, e.g., to induce and immune response to the tumor and/or to ablate any cancer cells left behind after resection.

Such methods can include providing a microneedle comprising a therapeutic agent, such as an anti-cancer agent, an immunomodulatory agent, or a combination thereof described herein. For example, such methods can include providing at least one microneedle or at least one microneedle device described herein, wherein the microneedle or the microneedle device comprises a silk fibroin-based tip having a therapeutic agent, such as an anti-cancer agent, an immunomodulatory agent, or a combination thereof; causing the microneedle or microneedle device to penetrate into the biological barrier (e.g., the skin, e.g., tumor); and allowing an effective amount of therapeutic agent to be released from the silk fibroin tips over a period of time, e.g., at least about 1 days (e.g., about 1, 2, 3, 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks, e.g., 1, 2, 3, 4, 5, 6, 7, 8, 9, 10, 11 months, e.g., 1, 2, 3, 4, 5 years, or longer).

### Combination Therapies

The microneedles disclosed herein can be used in combination with a second therapeutic agent or procedure.

In embodiments, the microneedle disclosed herein and the second therapeutic agent or procedure are administered/performed after a subject has been diagnosed with a cancer, e.g., before the cancer has been eliminated from the subject. In embodiments, the microneedle and the second therapeutic agent or procedure are administered/performed simultaneously or concurrently. For example, the delivery of one treatment is still occurring when the delivery of the second commences, e.g., there is an overlap in administration of the treatments. In other embodiments, the microneedle and the second therapeutic agent or procedure are administered/performed sequentially. For example, the delivery of one treatment ceases before the delivery of the other treatment begins.

In embodiments, combination therapy can lead to more effective treatment than monotherapy with either agent alone. In embodiments, the combination of the first and second treatment is more effective (e.g., leads to a greater reduction in symptoms and/or cancer cells) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the microneedle molecule is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the microneedle and the therapy, e.g., the cancer therapy, can be sequential (with or without overlap) or simultaneous. Administration of the microneedle can be continuous or intermittent during the course of therapy (e.g., cancer therapy). Certain therapies described herein can be used to treat cancers and non-cancerous diseases. For example, the efficacy of a therapeutic agent can be enhanced in cancerous and non-cancerous conditions using the methods and compositions described herein.

### Patient Selection

In some embodiments of any of the methods of treating a subject, or composition for use disclosed herein, the subject has a disorder, for example, a cancer.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors. Examples of various cancers are described herein and include but are not limited to, an anal cancer; a basal cell carcinoma; a bladder cancer; a bone cancer; a brain tumor; a breast cancer; a cervical cancer; a colon and rectal cancer; a endometrial cancer; an esophageal cancer; a gastrointestinal cancer (e.g., a gastrointestinal stromal tumor); a gestational trophoblastic disease; a head and neck cancer; a Hodgkin lymphoma; a Kaposi sarcoma; a kidney (renal cell) cancer; a leukemia; a liver cancer; a lung cancer; a malignant mesothelioma; a melanoma; a Merkel cell carcinoma; a multicentric Castleman disease; a multiple myeloma and other plasma cell neoplasms; a myeloproliferative neoplasms; a neuroblastoma; a Non-Hodgkin lymphoma; a ovarian, fallopian tube, or primary peritoneal cancer; a pancreatic cancer; a penile cancer; a pheochromocytoma and paraganglioma; a prostate cancer; a retinoblastoma; a rhabdomyosarcoma; a skin cancer; a squamous cell carcinoma; a soft tissue sarcoma; a solid tumor anywhere in the body; a stomach (gastric) cancer; a testicular cancer; a thyroid cancer; a vaginal cancer; a vulvar cancer; and a Wilms tumor and other childhood kidney cancers; and the like.

In some embodiments, the cancer is a melanoma. In some embodiments, the cancer is a basal cell carcinoma. In some embodiments, the cancer is a squamous cell carcinoma. In some embodiments, the cancer is a Merkel cell carcinoma. In some embodiments, the cancer is a breast cancer.

In some embodiments of any of the methods of treating a subject, or composition for use disclosed herein, the subject has a skin condition. Examples of various skin conditions are described herein and include but are not limited to, actinic keratosis (AK), lentigo maligna, leukoplakia, and Bowen's Disease.

### Exemplary Kits

In certain embodiments, the present disclosure relates to a package or kit comprising a microneedle described herein. In some embodiments, the present disclosure relates to a package or kit comprising a therapeutic agent described herein. In some embodiments, the kit can further comprise an additional therapeutic for combination therapy with the microneedle. In some embodiments, the kits can further comprise a disinfectant (e.g., an alcohol swab). In some embodiments, the kits can further comprise instructions (e.g., instructions useful for the application or administration of a microneedle device described herein). In some embodiments, such packages, and kits described herein can be used for vaccination purposes, e.g., to achieve broad-spectrum immunity in a subject as described herein. In some embodiments, such packages, and kits described herein can be used for cancer treatment or prevention purposes, e.g., to treat or prevent cancer in a subject, as described herein.

### Vaccines

The microneedles and methods described herein can also be used in the delivery of a vaccine to a subject in need thereof. With respect to vaccine delivery, the present disclosure is based, at least in part, on the discovery that modulating the kinetics of antigen presentation via, e.g., controlled- and/or sustained release compositions and devices (e.g., microneedles, e.g., silk-based microneedles, and microneedles devices) comprising a vaccine as described herein, e.g., a viral vaccine such as an influenza vaccine, can drive a more potent and/or lasting immune response (e.g., a more potent and/or lasting cellular immune response and/or humoral immune response) in a subject, e.g., as compared to the administration of single-dose or bolus administration of the vaccine. In some embodiments, controlled- or sustained-release of a vaccine as described herein can be used to achieve broad spectrum immunity in a subject.

In some embodiments, the microneedles and microneedles devices described herein demonstrate controlled- or sustained-release of a vaccine (e.g., an influenza vaccine) for at least about 1-2 weeks (e.g., for at least about 1, 2, 3, 4, 5, 6, 7, 8, 9, 10, 11, 12, 13 or 14 days), which results in one or more of improved immunogenicity, an enhanced immune response, and/or broad-spectrum immunity.

In certain embodiments, microneedle of the present disclosure can be configured to achieve the controlled- or sustained-release of a vaccine, an antigen, and/or an immunogen as described herein (e.g., an influenza vaccine). Without wishing to be bound by theory the administration of a microneedle disclosed herein comprising a vaccine can induce the development of a broad-spectrum immunity to the virus in a subject.

Non-limiting examples of vaccines for use in the microneedles and microneedle devices (e.g., microneedle patches) described herein can include a commercial vaccine, such as a seasonal vaccine, a pandemic vaccine, and/or a universal vaccine; egg-based vaccines, cell-culture based vaccines; recombinant vaccines; live attenuated, inactivated whole virus, split virion, and/or protein subunit vaccines; and adjuvanted vaccines.

As used herein, the term “viruses” refers to an infectious agent composed of a nucleic acid encapsidated in a protein. Such infectious agents are incapable of autonomous replication (i.e., replication requires the use of the host cell's machinery). Viral genomes can be single-stranded (ss) or double-stranded (ds), RNA or DNA, and can or cannot use reverse transcriptase (RT). Additionally, ssRNA viruses can be either sense (+) or antisense (−). Exemplary viruses include, but are not limited to, dsDNA viruses (e.g., Adenoviruses, Herpesviruses, Poxviruses), ssDNA viruses (e.g., Parvoviruses), dsRNA viruses (e.g., Reo viruses), (+)ssRNA viruses (e.g., Picomaviruses, Toga viruses), (−)ssRNA viruses (e.g., Orthomyxoviruses, Rhabdoviruses), ssRNA-RT viruses, i.e., (+)sense RNA with DNA intermediate in life-cycle (e.g., Retroviruses), and dsDNA-RT viruses (e.g., Hepadnaviruses). In some embodiments, viruses can also include wild-type (natural) viruses, killed viruses, live attenuated viruses, modified viruses, recombinant viruses or any combinations thereof. Exemplary retroviruses include human immunodeficiency virus (HIV). Other examples of viruses include, but are not limited to, enveloped viruses, respiratory syncytial viruses, non-enveloped viruses (e.g., human papillomavirus (HPV)), bacteriophages, recombinant viruses, and viral vectors. The term “bacteriophages” as used herein refers to viruses that infect bacteria.

As an example, various commercial influenza vaccines which can be incorporated into a microneedle of the present disclosure are listed below. Additionally, influenza vaccines comprising an mRNA, a DNA, a viral vector, and/or a virus-like particle (VLP) are suitable for use in the microneedles and microneedle devices (e.g., microneedle patches) described herein. In some embodiments, the influenza vaccine may target matrix protein 1, matrix protein 2 (M2e), and/or nucleoprotein (NP) of an influenza virus.

| | |
|---|---|
| Vaccine | Manufacturer |

Seasonal Influenza Vaccines

| | |
|---|---|
| Fluzone High Dose | Sanofi Pasteur |
| Fluzone Quadrivalent | Sanofi Pasteur |
| Fluzone Intradermal Quadrivalent | Sanofi Pasteur |
| Afluria/Fluvax | Seqirus |
| Agriflu | Seqirus |
| Fluad | Seqirus |
| Flucelvax | Seqirus |
| Fluvirin | Seqirus |
| Aggripal | Seqirus |
| FluMist Quadrivalent | MedImmune |
| Flublok | Protein Sciences (Sanofi Pasteur) |
| FluLaval | GlaxoSmithKline |
| Fluarix | GlaxoSmithKline |
| Influvac | Mylan |
| Preflucel | Nanotherapeutics |
| Anflu | Sinovac Biotech |

Pandemic Influenza Vaccines

| | |
|---|---|
| Influenza Virus Vaccine, H5N1 | Sanofi Pasteur |
| Pandemrix | GlaxoSmithKline |
| Panflu | Sinovac Biotech |
| Panflu 1 | Sinovac Biotech |

### EXAMPLES

The present disclosure is further described in detail by reference to the following experimental examples. These examples are provided for purposes of illustration only, and are not intended to be limiting unless otherwise specified. Thus, the present disclosure should in no way be construed as being limited to the following examples, but rather, should be construed to encompass any and all variations which become evident as a result of the teaching provided herein.

### Example 1. In Vivo Evaluation of the Efficacy of Silk-Based Microneedle Administration of Anti-Cancer Agents and Immunomodulatory Agents for the Treatment of Solid Tumors/Cancer

An in vivo model of mouse melanoma can be used to assess the efficacy of sustained release of multiple therapeutic agents (e.g., cytokines, checkpoint inhibitors, chemotherapeutics, mRNA encoding for anti-cancer agents or a combination of two, three, or all four) in their ability to control the tumor microenvironment. While the model used in these experiments is melanoma, it is expected that this platform has wider uses in other cancers, including solid cancers.

Mouse melanoma cells (B16-F10) are to be injected into the flank (right side) of mice (C57Bl/6) and a tumor is to be allowed to form. The agent will be administered intratumorally or peritumorally (i.e., intradermally or subcutaneously) either as:
1. a single bolus,
2. a series of fractional injections where the total dose given over the time frame is equivalent to the single bolus. These injections may be given daily or every other day.
3. a silk microneedle patch formulated with the one or more therapeutic agents.
   The therapeutic agents to be investigated (i.e., as single agents and in combination) in these experiments may include, but are not limited to:
Anti-cancer agents, such as:
   Chemotherapeutic agents: Gemcitabine, Doxorubicin, Oxaliplatin, Dacarbazine, Temozolomide
   Targeted therapies: Vemurafenib, Dabrafenib, Trametinib
   Other: mRNA encoding for anti-cancer agents
Immunomodulatory agents, such as:
   Cytokines: IL-2, IL-12, IL-15, GM-CSF
   Immunomodulatory agents: CpG, c-di-GMP
   Checkpoint inhibitors: anti-PD1, anti-PD-L1, anti-CTLA4
   Other: mRNA encoding for cytokines and other immunomodulatory molecules

The duration of sustained release (daily injections or silk microneedles) will be explored and optimized (˜2-28 days). Additionally, it is possible that multiple cycles of sustained released are optimal for tumor clearance. To test this, the agents will be administered via either single bolus or sustained release (daily injections or silk microneedles) over ˜2-28 days. For a period of time (˜5-14 days), animals will be given no treatment and then given the agent in a second round identical to the first.

Tumor growth will be measured two to three times weekly. Animals will be euthanized when humane endpoints have been reached. These endpoints include weight loss >15%, body condition score <1, tumor volume >1 cm³, tumor length (defined as the longest dimension) >1.5 cm, or tumor ulceration/necrosis. These humane endpoints apply to all in vivo experiments using the melanoma model.

Sustained release of therapeutic agents may lead to inhibition of tumor growth (compared to conventional single bolus injection) or total clearance of the tumor. These experiments will identify optimal dosing kinetics, timing, and administration route. Importantly, it is an aim to downselect and identify an agent or combination of agents that have an effect on tumor growth. The test these agents' ability to enhance the abscopal effect and memory responses will be tested (experiments detailed below).

### Intratumoral Administration of IL-2 or Gemcitabine in B16-F10 Mice

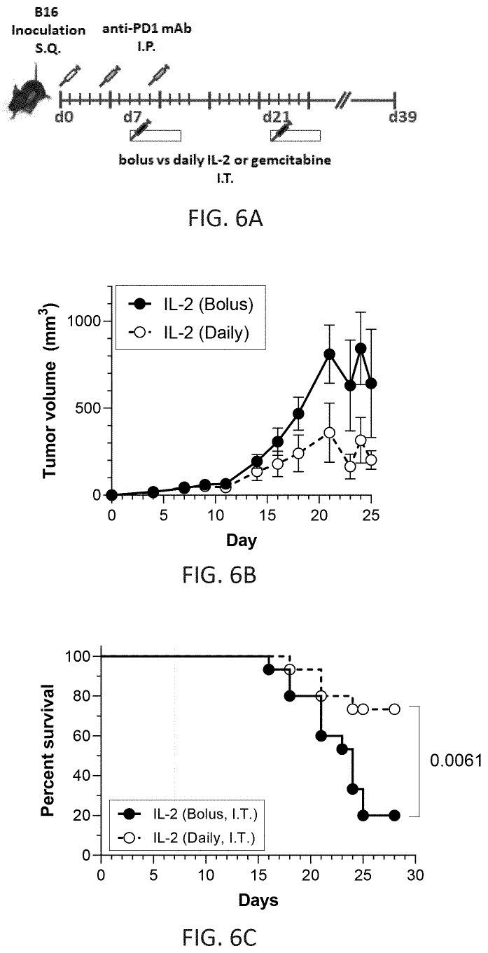

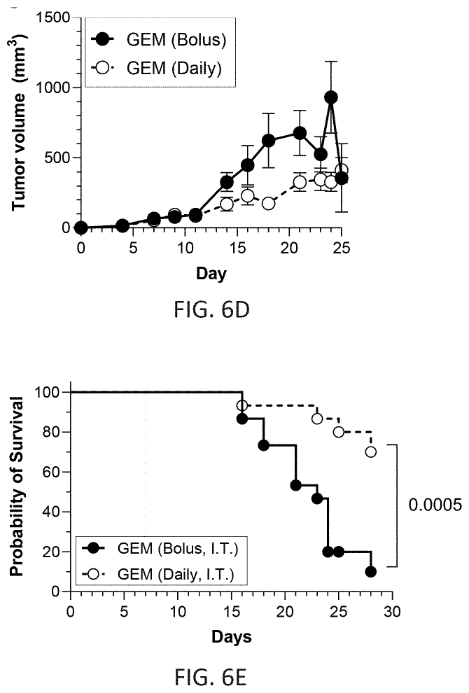

According to the methods outlined above, daily intratumoral administration was compared to single intratumoral bolus doses, with IL-2 or gemcitabine, using a B16-F10 mouse melanoma tumor model. For this study, two cycles of treatment were performed, including a bolus administration on Day 7 and on Day 21, and daily administration on Days 7-11 and Days 21-25. (see FIG. 6A). On Day 0, mice were inoculated by subcutaneous administration of B16-F10 cells. On day 4 and Day 9, mice were administered a dose of an anti-PD1 mAb (100 ug per dose) by intraperitoneal injection. Data was pooled from two experiments, with 15 mice per group.

First Cycle: On Day 7, mice in the ‘single bolus dose’ group received a single bolus dose of IL-2 (5 μg) or gemcitabine (475 μg). Mice in the ‘daily dose’ group received daily doses (intratumoral) of IL-2 (1 μg) or gemcitabine (95 μg) over Days 7-11 (5 total doses). Each Daily doses was a fractional dose equivalent to approximately 1/5 of the single bolus dose.

Second Cycle: On Day 21, mice in the ‘single bolus dose’ group received a single bolus dose of IL-2 (5 μg) or gemcitabine (475 μg). Mice in the ‘daily dose’ group received daily doses (intratumoral) of IL-2 (1 μg) or gemcitabine (95 μg) over Days 21-25 (5 total doses). Each daily doses was a fractional dose equivalent to approximately 1/5 of the single bolus dose.

B16-F10 mice that received daily doses of IL-2 had lower tumor burden compared to the counterpart mice receiving the bolus doses, as determined by tumor volume over time (see FIG. 6B; mean+SEM). The mice that received daily doses of IL-2 also had better survival than the mice that received bolus dose of IL-2, as demonstrated by the Kaplan-Meier curve shown in FIG. 6C.

Similarly, B16-F10 mice treated with daily intratumoral doses of gemcitabine exhibited a lower tumor burden compared to the equivalent mice that received bolus gemcitabine, as determined by tumor volume over time (see FIG. 6D; mean+SEM). The mice that received daily intratumoral gemcitabine had a greater survival compared to the bolus group, as illustrated by the Kaplan-Meier curve provided in FIG. 6E.

### Intratumoral Administration of Gemcitabine in CT26 Mice

Daily intratumoral administration compared to single intratumoral bolus doses was further investigated using gemcitabine in a murine CT26 colon carcinoma model (see FIG. 7A-7B). On day 0, mice were inoculated by subcutaneous administration of CT26 cells. On day 4 and day 9, mice were administered a dose of an anti-PD1 mAb (100 μg per dose) by intraperitoneal injection. Data was pooled from two experiments, with 10 mice per group.

CT26 mice in the ‘single bolus dose’ group received a single bolus dose of gemcitabine (475 μg) (intratumoral) at day 7, and four doses of saline (intratumoral) for the next four days (FIG. 7A). CT26 mice in the ‘daily dose’ group received daily doses of gemcitabine (95 μg) (intratumoral) over days 7-11 (5 total doses) (FIG. 7B). Each daily doses was a fractional dose equivalent to approximately ⅕ of the single bolus dose. Analysis of tumor volume over time revealed that the mice in the daily dose group had a lower tumor burden compared to the single bolus group (see FIG. 7C). The overall survivability of the mice in the daily dose group was also greater than the bolus dose group, as evidenced by the Kaplan-Meier survival curve provided in FIG. 7D.

### Example 2. In Vivo Assessment of Abscopal Effect in Distal Tumors

To determine if sustained release generates an abscopal effect, the phenomenon where local treatment of a tumor can inhibit further growth or promote clearance of a distal tumor, tumors will be induced in both flanks (right and left sides) of mice using B16-F10 melanoma cells. When a tumor has developed, the agent(s) will be administered) into the right tumor as:
1. a single bolus
2. a series of fractional dose injections
3. a silk microneedle patch formulated with the agent/s.

Growth of both tumors will be measured over time, two to three times a week. Animal will be euthanized when human endpoints have been reached. If sustained release of the agent(s) promotes the abscopal effect, the left (untreated) tumor should be significantly smaller than those of single bolus-treated animals.

### Example 3. In Vivo Assessment of the Ability to Establish Memory Responses that Will Lead to Rejection of Future Tumors

To determine if a superior immunological memory response is established upon primary tumor treatment, tumors will be induced in a single flank (right side) of mice using B16-F10 melanoma cells. The agent(s) will be administered as:
1. a single bolus
2. a series of fractional dose injections
3. a silk microneedle patch formulated with the agent/s.

Tumor size will be measured over time. After an extended period of time (˜60-90 days), cells will be injected into the flank (left) opposite that of the initial tumor. The size of the new tumor will be tracked over time. If sustained release of the agent(s) enhances the anti-cancer memory response, new tumors should fail to grow or be significantly smaller in volume than those of conventionally treated animals.

### Example 4. In Vivo Evaluation of Sustained Release of Cytokines/Chemotherapeutic Agents/Checkpoint Inhibitors

For protein-based agents (e.g. cytokines, anti-checkpoint antibodies), agents will be labeled with a fluorophore. If a combination of agents is used, agents will be labeled with different fluorophores. Tumors will be induced in mice using the B16-F10 model as previously described. Treatment will be administered intratumorally or peritumorally via:
1. single bolus
2. a silk microneedle patch formulated with the agent/s.

The duration of agent release will be visualized using In Vivo Imaging System (IVIS). Agents administered via bolus will be rapidly cleared from the tumor, while agents from the silk microneedles should be present within the tumor for extended durations (several days to weeks).

Chemotherapeutics are generally small molecules that cannot be labelled with a bulky fluorophore. To determine sustained release of these formulations using IVIS, a surrogate molecule similar in solubility, size, and charge to the agent(s) that can be visualized (e.g. rhodamine B, coumarin 6) will be administered to tumors as previously described.

### Example 5. In Vitro Evaluation of the Efficacy of Silk-Based Microneedle Administration of Cytokines for the Treatment of Melanoma

To determine that the cytokine cargo of the silk-based microneedles is released and biologically functional, a series of in vitro assays will be used.

For all cytokines (e.g. IL-2, IL-12, IL-15, GM-CSF), commercially available enzyme-linked immunosorbent assays (ELISAs) will be used to determine the amount of cytokine released from microneedles.

To evaluate if biofunction is conserved, cell line-based assays will be employed. For select cytokines, cell lines whose growth are dependent upon these cytokines are available (e.g. CTLL-2, HT-2). Proliferation of the cells will be measured over several (2-5) days using a tetrazolium salt (e.g. WST-1, MTT, or MTS).

IL-12 function may be measured via the production of IFNγ by mouse splenocytes. IFNγ will then be measured using ELISA.

The biofunction of most cytokines can be determined using commercially available reporter cell lines, which are genetically engineered to produce a measurable protein (colorimetric, luminometric or fluorescent) in a dose-dependent manner.

In all of these experiments, the concentration of functional cytokine released from the microneedles will be calculated by comparing the cellular readout to a standard curve. The effect of silk itself on each of these assays will be assessed by performing these assays with and without purified silk (at concentrations within the range that microneedles will be formulated).

### Stability of IL-2 in Dried Silk Compositions

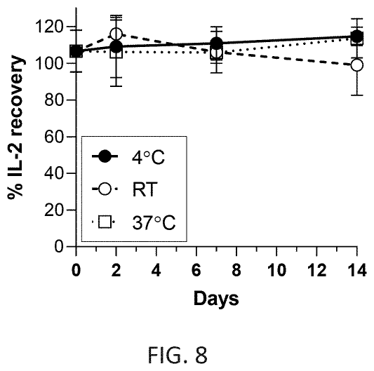

To investigate the stability of interleukin 2 (IL-2) over time in a silk format, silk films were generated using 2% silk and 10 ng/mL IL-2, and were air dried on PDMS. The films were then maintained at either 4° C., room temperature, or 37° C. for a period of 14 days. The films were then reconstituted in complete cell culture medium, prior to adding to HEK-Blue IL-12 (Invivogen) cells, which secrete alkaline phosphatase only in the presence of biofunctional IL-2. Release of alkaline phosphatase was then measured after 24 hours, the results of which are provided in FIG. 8. FIG. 8 depicts a cell-based assay, which can have a certain amount of variation. The standard curves used to calculate recovery included silk to exclude assay variation as a factor for higher recovery values. Overall, these results demonstrate that the stability and activity of IL-2 is maintained over time in dried silk compositions.

### Example 6. In Vitro Evaluation of the Efficacy of a Silk-Formulated Small Molecule Chemotherapy for the Treatment of Melanoma

Tumor cell viability will be measured over a 5-day period to assess the effective duration of a single dose of chemotherapeutic small molecule in vitro. Treatments will be prepared by casting the small molecule chemotherapy (e.g. gemcitabine, doxorubicin, oxaliplatin) with or without formulation onto polydimethylsiloxane (PDMS) disks. Treatments will be dried for 4 hours at 10% relative humidity, before being transferred into microtubes. Melanoma cells (e.g. B16-F10) will be seeded into 24-well plates at a density of 10,000 cells/well and incubated. After 24 hours, fresh media will be used to reconstitute treatment films and will be used to replace the media in the 24-well plates. Cell viability will be measured daily via a tetrazolium salt assay. To assess the effects of formulation on cell viability, treatment groups will include an untreated control (saline), formulation only group (no small molecule), small molecule without formulation, and small molecule dried in the presence of formulation. It is expected that the results of the cell viability assay will show that chemotherapy agents formulated in silk maintain cytotoxic functionality.

### EQUIVALENTS

While this disclosure has been particularly shown and described with references to preferred embodiments thereof, it will be understood by those skilled in the art that various changes in form and details may be made therein without departing from the spirit and scope of the present disclosure as defined by the appended claims. Those skilled in the art will recognize, or be able to ascertain using no more than routine experimentation, many equivalents to the specific embodiments of the present disclosure described specifically herein. Such equivalents are intended to be encompassed in the scope of the appended claims.

1. A microneedle device comprising a plurality of silk fibroin-based microneedles, wherein said plurality of microneedles comprises:
a first microneedle comprising an anti-cancer agent; and
a second microneedle comprising an immunomodulatory agent,
wherein the first and/or second microneedles comprises a silk fibroin, and the microneedle device is configured to deliver to the subject the anti-cancer agent and the immunomodulatory agent.

2. The microneedle device of claim 1, wherein the first and/or second microneedle in the plurality of microneedles comprises:
(i) a dissolvable base,
(ii) an implantable silk fibroin tip comprising a silk fibroin applied to the base, and
(iii) a backing applied to the base.

3. (canceled)

4. The microneedle device of claim 2, wherein:
(i) the silk fibroin tip comprises the anti-cancer agent and/or the immunomodulatory agent; and/or
(ii) the base comprises the anti-cancer agent and/or the immunomodulatory agent.

5. (canceled)

6. (canceled)

7. The microneedle device of claim 1, further comprising a third microneedle, wherein the third microneedle comprises an anti-cancer agent and/or an immunomodulatory agent.

8. The microneedle device of claim 1, which is configured to deliver two or more anti-cancer agents, wherein the two or more anti-cancer agents are in the same or different microneedles.

9.-10. (canceled)

11. The microneedle device of claim 1, which is configured to deliver two or more immunomodulatory agents, wherein the two or more immunomodulatory agents are in the same or different microneedles.

12.-13. (canceled)

14. The microneedle device of claim 1, wherein the anti-cancer agent and the immunomodulatory agent are in the same or different microneedles.

15. (canceled)

16. The microneedle device of claim 1, wherein the anti-cancer agent is one or more of a small molecule, a biologic, a viral cancer therapeutic agent, a nanopharmaceutical, or a nucleic acid molecule.

17. The microneedle device of claim 1, wherein the anti-cancer agent is an mRNA.

18. The microneedle device of claim 1, wherein the immunomodulatory agent is selected from the group consisting of a checkpoint inhibitor, a Toll-like receptor (TLR) agonist, a STING agonist, a RIG agonist, a cancer vaccine, and a cytokine.

19. The microneedle device of claim 18, wherein:
(i) the checkpoint inhibitor inhibits a checkpoint molecule selected from the group consisting of CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR;
(ii) the TLR agonist is selected from the group consisting of a TLR-1 agonist, a TLR-2 agonist, a TLR-3 agonist, a TLR-4 agonist, a TLR-5 agonist, a TLR-6 agonist, a TLR-7 agonist, a TLR-8 agonist, a TLR-9 agonist, a TLR-10 agonist, a TLR-1/2 agonist, a TLR-2/6 agonist, or a TLR-7/8 agonist;
(iii) the STING agonist comprises a cyclic dinucleotide; and/or
(iv) the cytokine is selected from the group consisting of GM-CSF, IL-1α, IL-1, IL-2, IL-3, IL-4, IL-5, IL-6, IL-7, IL-8, IL-10, IL-12, IL-15, IL-18, IL-21, IFN-α, IFN-β, IFN-γ, MIP-1α, MIP-1β, TGF-β, TNF-α, and TNFβ.

20.-22. (canceled)

23. The microneedle device of claim 1, which is configured to administer an anti-PD1 antibody and/or an anti-CTLA4 antibody in combination with one or more of:
(i) IL-2;
(ii) IL-12;
(iii) IL-15;
(iv) IL-18;
(v) Gemcitabine (GEMZAR®);
(vi) Vemurafenib (ZELBORAF®);
(vii) Dabrafenib (TAFINLAR®);
(viii) Trametinib (MEKINIST®);
(ix) Doxorubicin (ADRIAMYCIN®);
(x) c-di-GMP;
(xi) mRNA;
(xii) a TLR-9 agonist (e.g., an unmethylated CG dinucleotides (CpG ODN));
(xiii) oxaliplatin; or
(xiv) GM-CSF.

24. The microneedle device of claim 1, wherein:
(i) the device is configured for sustained release of the anti-cancer agent and/or the immunomodulatory agent, and wherein the sustained release is over a period of time comprising at least about 2, 3, 4, 5, 6, 7, or more days;
(ii) the device is configured for burst release of the anti-cancer agent and/or the immunomodulatory agent, and wherein the burst release is over a period of time comprising at least about 1 hour, and/or
(iii) the release of the anti-cancer agent occurs at a different rate than the release of the immunomodulatory agent, such that anti-cancer agent is released substantially before or substantially after the release of the immunomodulatory agent.

25.-26. (canceled)

27. The microneedle device of claim 1, wherein:
(i) the device is configured to be applied to a biological barrier selected from the group consisting of a layer of skin, a cell membrane, a mucous surface, an oral cavity, or a buccal cavity, and wherein the microneedle is configured to pierce the biological barrier;
(ii) the device is configured to be applied to a tumor;
(iii) the device is configured to be applied to the site of a tumor after tumor resection;
(iv) the device is configured to be applied to the site of a tumor before tumor resection;
(v) the device is configured to be applied intratumorally;
(vi) the device is configured to be applied peritumorally; and/or
(vii) the device is configured to be applied to, or proximal to, a skin lesion associated with a cancer or a precancerous condition.

28.-33. (canceled)

34. The microneedle device of claim 1, wherein the device is configured for local and/or systemic delivery which results in:
(i) inhibition of tumor growth at or near the site of administration;
(ii) an induction of a local immune response to ablate tumors at or near the site of administration;
(iii) an increase in activated immune effector cells in the tumor microenvironment;
(iv) a reduction in local immunosuppressive cells
(v) an induction of a systemic immune response to ablate tumors at distant sites;
(vi) an immunological memory to the cancer or precancerous condition; and/or
(vii) an immune response to a tumor antigen, such as a neoantigen; and/or
(viii) prevention and/or inhibition of cancer recurrence.

35. The microneedle device of claim 2, wherein:
(i) the backing is chosen from a solid support comprising a paper-based material, a plastic material, a polymeric material, or a polyester-based material; and/or
(ii) the dissolvable base comprises two or more of:
a polysaccharide;
a disaccharide;
a polymer;
a protein;
a plasticizer; and/or
a surfactant.

36. (canceled)

37. The microneedle device of claim 35, wherein the base comprises one or more of gelatin, dextran, glycerol, polyethylene glycol (PEG), sucrose, trehalose, maltose, carboxymethylcellulose (CMC), polyvinylpyrrolidone (PVP), polyvinyl alcohol (PVA), hyaluronate, methyl cellulose, and/or a surfactant.

38. The microneedle device of claim 2, wherein:
(i) the silk fibroin tip comprises two or more of:
a disaccharide;
a polymer;
an amino acid;
a plasticizer; and/or
a buffer;
(ii) the silk fibroin tip comprises one or more of carboxymethylcellulose (CMC), methyl cellulose, polyethylene glycol (PEG), polyvinylpyrrolidone (PVP), polyvinyl alcohol (PVA), hyaluronate, sucrose, maltose, trehalose, glycerol, propanediol, PBS, or threonine;
(iii) the silk fibroin tip further comprises an excipient; and/or
(iv) the silk fibroin tip comprises about 1% w/v to about 10% w/v of silk fibroin.

39.-46. (canceled)

47. A method for treating a cancer and/or inducing an immune response to a cancer, comprising contacting a microneedle device; or of claim 1 to the site of a cancer of a subject.

48.-63. (canceled)

64. A method of producing a microneedle device, the method comprising:
providing a mold including a mold body with an array of needle cavities having a predefined shape formed therein;
filling tips of the needle cavities with a composition comprising a silk fibroin, and a therapeutic agent solution;
drying the filled tips of the needle cavities to create silk fibroin tips, and optionally annealing the silk fibroin tips;
further filling the needle cavities of the mold with a first base solution;
drying the first base solution to form a first base layer, thereby producing a microneedle device.
